SEQUENCE LISTING (52 sequences)

SEQ ID NO: 1 - DNA, 19 bp, synthetic construct
  misc_feature 1..19: note = Wild-type T7 promoter
  source 1..19: mol_type = other DNA
taatacgactcactatagg

SEQ ID NO: 2 - DNA, 19 bp, synthetic construct
  misc_feature 1..19: note = Synthetic - RV mutant T7 promoter
  source 1..19: mol_type = other DNA
taataaccctcactatagg

SEQ ID NO: 3 - DNA, 19 bp, synthetic construct
  misc_feature 1..19: note = Synthetic - AKSIRV mutant T7 promoter
  source 1..19: mol_type = other DNA
taatacctgacactatagg

SEQ ID NO: 4 - DNA, 19 bp, synthetic construct
  misc_feature 1..19: note = Synthetic - IRH mutant T7 promoter
  source 1..19: mol_type = other DNA
taataactatcactatagg

SEQ ID NO: 5 - DNA, 19 bp, synthetic construct
  misc_feature 1..19: note = Synthetic - KIRV mutant T7 promoter
  source 1..19: mol_type = other DNA
taataccggtcactatagg

SEQ ID NO: 6 - DNA, 35 bp, Escherichia coli
  misc_feature 1..35: note = J23119 promoter
  source 1..35: mol_type = other DNA
ttgacagctagctcagtcctaggtataatactagt

SEQ ID NO: 7 - DNA, 63 bp, Escherichia coli
  misc_feature 1..63: note = Promoter for ArsR EP3 mutant
  source 1..63: mol_type = other DNA
acacattcgttaagtcatatatgtttttgacttatccgcttcgaagagatataatacctgcaa

SEQ ID NO: 8 - DNA, 44 bp, Escherichia coli
  misc_feature 1..44: note = Promoter for MerR
  source 1..44: mol_type = other DNA
atcgcttgactccgtacatgagtacggaagtaaggttacgctat

SEQ ID NO: 9 - DNA, 115 bp, Escherichia coli
  misc_feature 1..115: note = Promoter for NarX and Nar L (pydfJ115 hybrid)
  source 1..115: mol_type = other DNA
actgcatatttgaaaattgcccaaacgtacatgcccgaatgtacgtttttttcatttcattgtcaactacaatgagaaagaatgtgatcaagcaatgtgttgaaaggagattatc

SEQ ID NO: 10 - DNA, 39 bp, Escherichia coli
  misc_feature 1..39: note = Promoter for CueR
  source 1..39: mol_type = other DNA
ttcttgaccttccccttgctggaaggtttatcctcggtt

SEQ ID NO: 11 - DNA, 41 bp, Acinetobacter baumannii
  misc_feature 1..41: note = Promoter for PbrR
  source 1..41: mol_type = other DNA
atgtcttgactctatagtaactagagggtgttaaatcggca

SEQ ID NO: 12 - DNA, 117 bp, Agaricostilbomycetes sp.
  misc_feature 1..117: note = Promoter for crcB riboswitch
  source 1..117: mol_type = other DNA; note = TY-157
ttgacagctagctcagtcctaggtataatactagtttataggcgatggagttcgccataaacgctgcttagctaatgactcctaccagtatcactactggtaggagtctattttttt

SEQ ID NO: 13 - DNA, 41 bp, Pseudomonas entomophila
  misc_feature 1..41: note = Promoter for CadR
  source 1..41: mol_type = other DNA; strain = L48
ataacttgactctgtagttgctacagggtgtgcaatcggtt

SEQ ID NO: 14 - DNA, 34 bp, Ochrobactrum tritici
  misc_feature 1..34: note = Promoter for ChrB
  source 1..34: mol_type = other DNA; strain = 5bvl1
gtagatcttatctcattattgtagtaatatctac

SEQ ID NO: 15 - DNA, 2135 bp, synthetic construct
  misc_feature 1..2135: note = Synthetic - pT7-ArsR-EP3 plasmid
  source 1..2135: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggagaccacaacggtttccctctagaaataattttgtttaactttaagaaggagatatacataaagaggagaaatactagatgtggcaactaacaccacttcagttatttaaaaacctgtccgatgaaacccgtttgggtatcgtgttgttgctcagggagatgggagaattgtgcgtgtgtgatctttgcatggcactggatcaatcacagcccaaaatatcccgtcatctggcgatgctacgggtaagtggaatccttctggatcgtaaacagggaaaatgggttcactaccgcttatcaccccatattccttcatgggccgcccagattatagagcaggcctggttaagccaacaggacgacgttcaggtcatcgcacgcaagctggcttcagttaactgctccggtagcagtaaggctgtctgcatctaagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 16 - DNA, 2225 bp, synthetic construct
  misc_feature 1..2225: note = Synthetic - pT7-CadR plasmid
  source 1..2225: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggagaccacaacggtttccctctagaaataattttgtttaactttaagaaggagatatacataaagaggagaaatactagatgaaaatcggtgaacttgctaaagccacggactgtgcggttgaaactatccgttactacgagcgtgaacagttgctgccagagcctgcccgttcagacggtaattatcgcttatacacgcaggcacatgtggaacgtctgacgtttattcgcaactgccgtacattggatatgactcttgatgaaatccgttctctgttgcgtttgcgtgattcgccggatgatagctgcgggtccgtgaacgcgttaattgatgaacatattgagcatgtgcaagctcgtatcgatggactggttgcgcttcaggagcaactggtagaacttcgtcgtcgctgcaatgcccaaggtgctgagtgtgccatcttacagcaattggagacgaacggtgctgtttcggtccccgaaacagaacactcccatgtgggtcgctctcacggccactaggtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 17 - DNA, 2171 bp, synthetic construct
  misc_feature 1..2171: note = Synthetic - pT7-CueR plasmid
  source 1..2171: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggagaccacaacggtttccctctagaaataattttgtttaactttaagaaggagatatacatatgaatatctctgatgttgcaaagattacgggccttacctcgaaagcaatccgcttctacgaagaaaagggactggtcacacctcccatgcgtagcgaaaacggctaccgcacatacacccagcaacacctgaatgagctgactttgctgcgccaggcgcgtcaagtgggtttcaatttggaggaatccggggagctggtcaacctgttcaatgacccacaacgccattcggccgacgtgaaacgccgtacattagaaaaagttgccgaaattgaacgccatattgaagagctgcagtccatgcgcgatcagttgttagcactggcaaacgcttgtcctggagatgattcggctgattgtccgattatcgaaaacttatctggatgttgtcaccatcgcgcagggtaagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 18 - DNA, 2216 bp, synthetic construct
  misc_feature 1..2216: note = Synthetic - pT7-MerR plasmid
  source 1..2216: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggagaccacaacggtttccctctagaaataattttgtttaactttaagaaggagatatacataaagaggagaaatactagatggaaaataatttggaaaacctgaccattggcgtttttgccaaggcggccggggtcaacgtggagacaatccgcttctatcagcgcaagggcctgttgcgggaaccggacaagccttacggcagcatccgccgctatggggaggcggacgtggttcgggtgaaattcgtgaaatcggcacagcggctggggttcagtctggacgagattgccgagctgttgcggctcgacgatggcacccactgcgaggaggccagcagcctggccgaacacaagctcaaggacgtgcgcgagaagatggccgacttggcgcgcatggaaaccgtgctgtctgaactcgtgtgcgcctgccatgcacgaaaggggaatgtttcctgcccgttgatcgcgtcactacagggcgaagcaggcctggcaaggtcagctatgccttaggtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 19 - DNA, 2415 bp, synthetic construct
  misc_feature 1..2415: note = Synthetic - pT7-NarL-hybrid plasmid
  source 1..2415: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggagaccacaacggtttccctaaggattttttctagaagaattatatacaacaaatgagtaatcaggaaccggctactatcctgctgattgacgatcacccgatgctgcgaactggcgtaaaacagcttatcagtatggcaccagatatcaccgtggttggcgaagcgagtaatggcgaacagggtattgaactggcggagtctcttgatcccgatctgatcctgttagatctcaatatgcccggcatgaacggtctggaaacgctggataaactgcgcgaaaagtccctctcagggcgcattgtggtattcagcgtctctaaccatgaagaagatgtggtcaccgcactgaaacgcggcgcggatggctatctgttaaaagatatggaaccggaagatctgctgaaagcattgcatcaggcagctgctggcgaaatggtattaagccctgatatccttaaacgtctgcaagaaatccaatttgagcggatgaaaaagcagcgcaatgagacgcagctgacagaaaaggaagtcattgttctaaaagcaattgctaaaggtcttaaaagcaaagcgattgcctttgatttgggcgtctctgagcgaacagtaaagtccagattaacgtccatttacaataaattaggcgcgaattcaagaactgaggcagtaacgattgccatgcaaaaaggtattctgacaatagacaactaagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 20 - DNA, 3549 bp, synthetic construct
  misc_feature 1..3549: note = Synthetic - pT7-NarX plasmid
  source 1..3549: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggagaccacaacggtttcccgcactctcctaatttaaatcgcacaactcacatgcttaaacgttgtctctctccgctcaccctggttaatcaggttgcgcttattgtgttgctttctactgctattggactggcagggatggcggtttctggctggctggtgcaaggcgttcagggcagcgcccatgcgatcaacaaagcgggatcgctgcgcatgcaaagttaccgtctgttggcggcagtgccattaagcgagaaagacaagcccttaattaaagagatggaacaaacggcatttagcgccgagttgactcgagcagcagaacgagacggacaactggcgcaattacagggtttacaagattactggcgtaatgaactgatccctgcgctgatgcgtgcacaaaaccgagaaacggtgtcagcggatgtcagccagtttgttgccgggcttgatcaactggtatctggttttgaccgcaccacggaaatgcgcatcgagacagtggtactggtccatcgggtaatggcggtatttatggcacttttactggtgttcactattatctggttgcgggcgcgactgctacaaccgtggcggcaactgctggcaatggcgagtgccgtcagtcatcgcgattttacccaacgcgcaaacatcagcgggcgcaacgaaatggcgatgcttggaactgcgttgaacaatatgtctgcagaactggccgaaagttatgccgtacttgagcagcgggttcaggagaaaaccgccgggctggagcataaaaatcagatcctctcttttttatggcaggctaaccgccgtttgcattcccgcgccccgctgtgtgaacgcctgtcacctgtactcaacggcttacagaatttaaccctgctacgtgatatcgaattgcgggtgtatgacactgatgatgaagagaatcatcaggagtttacctgccagccagatatgacttgtgatgataaaggctgccagctctgcccgcgcggcgtattacccgttggtgatcgcggcacgaccctgaagtggcggctggctgactctcatacgcagtacggtattttgctggcgaccctgccgcaggggcgtcatcttagccatgatcaacaacaactggtggataccctggttgaacaactcaccgccacgctggcgctggatcgccatcaggaacgtcagcaacagttgatcgtgatggaagagcgtgccaccattgcgcgcgaactgcatgattctattgcccaatctctctcttgcatgaagatgcaggtgagttgtttacagatgcagggcgatgcgctgccagaaagcagccgcgaactgttaagtcagatccgtaacgaactgaatgcatcctgggcgcagttgcgtgaattgctcaccacattccgcttgcagctcaccgagcctggattacgtccggcgctggaggcgagttgcgaagagtacagcgccaaatttggcttcccggtgaagctggattatcaattgccgcctcgcctggtgccttcgcatcaggcaatccacttgttgcaaattgcccgtgaggcattaagtaacgccctcaaacattcgcaagcgagtgaagtcgtggtgacggtggcgcaaaacgataatcaggtcaaactgaccgtccaggataacggctgcggcgtgcctgaaaatgccatccgcagcaatcactacggcatgataataatgcgcgatcgtgcgcaaagtttacgaggcgattgccgcgtccgccgtcgtgaatcaggtggcaccgaagtggtggtcacctttattcccgaaaaaactttcacagacgtccaaggagatacccatgagtaagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 21 - DNA, 2201 bp, synthetic construct
  misc_feature 1..2201: note = Synthetic - pT7-PbrR plasmid
  source 1..2201: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggagaccacaacggtttccctctagaaataattttgtttaactttaagaaggagatatacatatgaacattcagatcggtgaactggcgaaacgtactgcatgtccagtggtgaccattcgcttttatgagcaagaaggtttgctgccgcccccaggacgttcccgtggtaatttccgcttatacggggaagagcatgttgagcgtttgcaatttatccgccattgccgctcacttgacatgcccttatccgatgtccgcacattgctgtcataccgcaagcgtcctgatcaagattgcggggaggtgaacatgcttctggatgagcatattcgtcaagtagagagtcgtattggcgctctgttagagttaaaacatcacttggtcgagctgcgcgaagcatgctcaggggctcgccctgcacaaagctgcggaatccttcagggcctgagtgattgtgtttgcgatacacgtggcacgacagcacacccctcggattaagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 22 - DNA, 2481 bp, synthetic construct
  misc_feature 1..2481: note = Synthetic - pAKSIRV-sfGFP plasmid
  source 1..2481: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacctgacactatagggagaccacaacggtttccctctagaaataattttgtttaactttaagaaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 23 - DNA, 2687 bp, synthetic construct
  misc_feature 1..2687: note = Synthetic - pAKSIRV-XylE plasmid
  source 1..2687: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacctgacactatagggagacaaagcccgccgaaaggcgggcttttttttgtttaactttaagaaggagatatacatatgaacaaaggtgtaatgcgaccgggccatgtgcagctgcgtgtactggacatgagcaaggccctggaacactacgtcgagttgctgggcctgatcgagatggaccgtgacgaccagggccgtgtctatctgaaggcttggaccgaagtggataagttttccctggtgctacgcgaggctgacgagccgggcatggattttatgggtttcaaggttgtggatgaggatgctctccggcaactggagcgggatctgatggcatatggctgtgccgttgagcagctacccgcaggtgaactgaacagttgtggccggcgcgtgcgcttccaggccccctccgggcatcacttcgagttgtatgcagacaaggaatatactggaaagtggggtttgaatgacgtcaatcccgaggcatggccgcgcgatctgaaaggtatggcggctgtgcgtttcgaccacgccctcatgtatggcgacgaattgccggcgacctatgacctgttcaccaaggtgctcggtttctatctggccgaacaggtgctggacgaaaatggcacgcgcgtcgcccagtttctcagtctgtcgaccaaggcccacgacgtggccttcattcaccatccggaaaaaggccgcctccatcatgtgtccttccacctcgaaacctgggaagacttgcttcgcgccgccgacctgatctccatgaccgacacatctatcgatatcggcccaacccgccacggcctcactcacggcaagaccatctacttcttcgacccgtccggtaaccgcaacgaagtgttctgcgggggagattacaactacccggaccacaaaccggtgacctggaccaccgaccagctgggcaaggcgatcttttaccacgaccgcattctcaacgaacgattcatgaccgtgctgacctaagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 24 - DNA, 2481 bp, synthetic construct
  misc_feature 1..2481: note = Synthetic - pIRH-sfGFP plasmid
  source 1..2481: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaataactatcactatagggagaccacaacggtttccctctagaaataattttgtttaactttaagaaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 25 - DNA, 2481 bp, synthetic construct
  misc_feature 1..2481: note = Synthetic - pKIRV-sfGFP plasmid
  source 1..2481: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaataccggtcactatagggagaccacaacggtttccctctagaaataattttgtttaactttaagaaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 26 - DNA, 2481 bp, synthetic construct
  misc_feature 1..2481: note = Synthetic - pRV-sfGFP plasmid
  source 1..2481: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaataaccctcactatagggagaccacaacggtttccctctagaaataattttgtttaactttaagaaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 27 - DNA, 2481 bp, synthetic construct
  misc_feature 1..2481: note = Synthetic - pT7-sfGFP plasmid
  source 1..2481: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaatacgactcactatagggagaccacaacggtttccctctagaaataattttgtttaactttaagaaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 28 - DNA, 5918 bp, synthetic construct
  misc_feature 1..5918: note = Synthetic - crcB-AKSIRV plasmid
  source 1..5918: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaagatctttgacagctagctcagtcctaggtataatactagtttataggcgatggagttcgccataaacgctgcttagctaatgactcctaccagtatcactactggtaggagtctatttttttggatctaggaggaaggatctatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgcgcattgggtaactcctgatggtttccctgtgtggcaggaatacaagaagcctattaaaacgcgcatctctataatgttcctcggtcagttcaccttaaagcctaccattaacaccaacaaagatagcgagattgatgcacgcaaacaggtgtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 29 - DNA, 5344 bp, synthetic construct
  misc_feature 1..5344: note = Synthetic - J23119-AKSIRV_low_copy plasmid
  source 1..5344: mol_type = other DNA
agttgtaattctcatgtttgacagcttatcatcgataagcttccgatggcgcgccgagaggctttacactttatgcttccggctgaattctaaagatctttgacagctagctcagtcctaggtataatactagtacgtcgactctcgagtgagattgttgacggtaccgtattttggatctaggaggaaggatctatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgcgcattgggtaactcctgatggtttccctgtgtggcaggaatacaagaagcctattaaaacgcgcatctctataatgttcctcggtcagttcaccttaaagcctaccattaacaccaacaaagatagcgagattgatgcacgcaaacaggtgtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactggatccttactcgagtctagactgcagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcctccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatata

SEQ ID NO: 30 - DNA, 5878 bp, synthetic construct
  misc_feature 1..5878: note = Synthetic - J23119-IRH plasmid
  source 1..5878: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaaagatctttgacagctagctcagtcctaggtataatactagtacgtcgactctcgagtgagattgttgacggtaccgtattttggatctaggaggaaggatctatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacataatgttcctcggtagtttcaacttacagcctaccgttaacaccaacaaagatagcgagattgatgcacgcaaacaggagtctggtatcgctcctaactttgtacacagccatgacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 31 - DNA, 5877 bp, synthetic construct
  misc_feature 1..5877: note = Synthetic - J23119-KIRV plasmid
  source 1..5877: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaagatctttgacagctagctcagtcctaggtataatactagtacgtcgactctcgagtgagattgttgacggtaccgtattttggatctaggaggaaggatctatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattaaaacgcgcgtccatataatgttcctcggtcagttcgaaatgcagcctaccattaacaccaacaaagatagcgagattgatgcacgcaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 32 - DNA, 5877 bp, synthetic construct
  misc_feature 1..5877: note = Synthetic - J23119-RV plasmid
  source 1..5877: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaagatctttgacagctagctcagtcctaggtataatactagtacgtcgactctcgagtgagattgttgacggtaccgtattttggatctaggaggaaggatctatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagaagcgcttggatatgattttcctcggtcagttccgcttacagcctaccattaacaccaacaaagatagcgagattgatgcacgcaaacaggtgtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 33 - DNA, 5905 bp, synthetic construct
  misc_feature 1..5905: note = Synthetic - pArs-AKSIRV plasmid
  source 1..5905: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaagatctacacattcgttaagtcatatatgtttttgacttatccgcttcgaagagatataatacctgcaaacgtcgactctcgagtgagattgttgacggtaccgtattttggatctaggaggaaggatctatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgcgcattgggtaactcctgatggtttccctgtgtggcaggaatacaagaagcctattaaaacgcgcatctctataatgttcctcggtcagttcaccttaaagcctaccattaacaccaacaaagatagcgagattgatgcacgcaaacaggtgtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 34 - DNA, 5884 bp, synthetic construct
  misc_feature 1..5884: note = Synthetic - pCad-AKSIRV plasmid
  source 1..5884: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaaagatctataacttgactctgtagttgctacagggtgtgcaatcggttacgtcgactctcgagtgagattgttgacggtaccgtattttggatctaggaggaaggatctatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgcgcattgggtaactcctgatggtttccctgtgtggcaggaatacaagaagcctattaaaacgcgcatctctataatgttcctcggtcagttcaccttaaagcctaccattaacaccaacaaagatagcgagattgatgcacgcaaacaggtgtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 35 - DNA, 5887 bp, synthetic construct
  misc_feature 1..5887: note = Synthetic - pMer-AKSIRV plasmid
  source 1..5887: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaaagatctatcgcttgactccgtacatgagtacggaagtaaggttacgctatacgtcgactctcgagtgagattgttgacggtaccgtattttggatctaggaggaaggatctatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgcgcattgggtaactcctgatggtttccctgtgtggcaggaatacaagaagcctattaaaacgcgcatctctataatgttcctcggtcagttcaccttaaagcctaccattaacaccaacaaagatagcgagattgatgcacgcaaacaggtgtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 36 - DNA, 5957 bp, synthetic construct
  misc_feature 1..5957: note = Synthetic - pNar-AKSIRV plasmid
  source 1..5957: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaagatctactgcatatttgaaaattgcccaaacgtacatgcccgaatgtacgtttttttcatttcattgtcaactacaatgagaaagaatgtgatcaagcaatgtgttgaaaggagattatcacgtcgactctcgagtgagattgttgacggtaccgtattttggatctaggaggaaggatctatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgcgcattgggtaactcctgatggtttccctgtgtggcaggaatacaagaagcctattaaaacgcgcatctctataatgttcctcggtcagttcaccttaaagcctaccattaacaccaacaaagatagcgagattgatgcacgcaaacaggtgtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 37 - DNA, 5041 bp, synthetic construct
  misc_feature 1..5041: note = Synthetic - pAKSIRV-AKSIRV plasmid
  source 1..5041: mol_type = other DNA
tcagtgcaatttatctcttcaaatgtagcacctgaagtcagccccatacgatataagttgtaattctcatgtttgacagcttatcatcgataagcttccgatggcgcgccgagaggctttaaacgccagcaacgcgatcccgcgaaattaatacctgacactatagggagaccacaacggtttccctctagaaataattttgtttaactttaagaaggagatatacatatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgcgcattgggtaactcctgatggtttccctgtgtggcaggaatacaagaagcctattaaaacgcgcatctctataatgttcctcggtcagttcaccttaaagcctaccattaacaccaacaaagatagcgagattgatgcacgcaaacaggtgtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagtcgaccggctgctaacaaagcccgaaaggaagctgagttggctgctgccaccgctgagcaataactagcataaccccttggggcctctaaacgggtcttgaggggttttttgctgaaagccaattctgaggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcctccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatt

SEQ ID NO: 38 - DNA, 4579 bp, synthetic construct
  misc_feature 1..4579: note = Synthetic - pAKSIRV-AKSIRV-LET plasmid
  source 1..4579: mol_type = other DNA
gtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttccgatggcgcgccgagaggctttaaacgccagcaacgcgatcccgcgaaattaatacctgacactatagggagaccacaacggtttccctctagaaataattttgtttaactttaagaaggagatatacatatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgcgcattgggtaactcctgatggtttccctgtgtggcaggaatacaagaagcctattaaaacgcgcatctctataatgttcctcggtcagttcaccttaaagcctaccattaacaccaacaaagatagcgagattgatgcacgcaaacaggtgtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtgagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttac

SEQ ID NO: 39 - DNA, 4448 bp, synthetic construct
  misc_feature 1..4448: note = Synthetic - pAKSIRV-RV plasmid
  source 1..4448: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgatcccgcgaaattaataaccctcactatagggagaccacaacggtttccctctagaaataattttgtttaactttaagaaggagatatacatatgagaggatcgcatcaccatcaccatcacggatccaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcattctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgcgcattgggtaactcctgatggtttccctgtgtggcaggaatacaagaagcctattaaaacgcgcatctctataatgttcctcggtcagttcaccttaaagcctaccattaacaccaacaaagatagcgagattgatgcacgcaaacaggtgtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtg

SEQ ID NO: 40 - DNA, 3205 bp, synthetic construct
  misc_feature 1..3205: note = Synthetic - crcB-fGFP plasmid
  source 1..3205: mol_type = other DNA
tgattctaaagatctttgacagctagctcagtcctaggtataatactagtttataggcgatggagttcgccataaacgctgcttagctaatgactcctaccagtatcactactggtaggagtctatttttttaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatacctagggtacgggttttgggatccttactcgagtctagactgcagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcctccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttccgatggcgcgccgagaggctttacactttatgcttccggct

SEQ ID NO: 41 - DNA, 3934 bp, synthetic construct
  misc_feature 1..3934: note = Synthetic - pArs-sfGFP plasmid
  source 1..3934: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaagatctacacattcgttaagtcatatatgtttttgacttatccgcttcgaagagatataatacctgcaaacgtcgactctcgagtgagattgttgacggtaccgtattttggatctaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 42 - DNA, 3913 bp, synthetic construct
  misc_feature 1..3913: note = Synthetic - pCad-sfGFP plasmid
  source 1..3913: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaaagatctataacttgactctgtagttgctacagggtgtgcaatcggttacgtcgactctcgagtgagattgttgacggtaccgtattttggatctaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgcaatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 43 - DNA, 3911 bp, synthetic construct
  misc_feature 1..3911: note = Synthetic - pCue-sfGFP plasmid
  source 1..3911: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaaagatctttcttgaccttccccttgctggaaggtttatcctcggttacgtcgactctcgagtgagattgttgacggtaccgtattttggatctaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgcaatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 44 - DNA, 3915 bp, synthetic construct
  misc_feature 1..3915: note = Synthetic - pMer-sfGFP plasmid
  source 1..3915: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaagatctatcgcttgactccgtacatgagtacggaagtaaggttacgctatacgtcgactctcgagtgagattgttgacggtaccgtattttggatctaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 45 - DNA, 3986 bp, synthetic construct
  misc_feature 1..3986: note = Synthetic - pNar-sfGFP plasmid
  source 1..3986: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaagatctactgcatatttgaaaattgcccaaacgtacatgcccgaatgtacgtttttttcatttcattgtcaactacaatgagaaagaatgtgatcaagcaatgtgttgaaaggagattatcacgtcgactctcgagtgagattgttgacggtaccgtattttggatctaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 46 - DNA, 3913 bp, synthetic construct
  misc_feature 1..3913: note = Synthetic - pPbr-sfGFP plasmid
  source 1..3913: mol_type = other DNA
tgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaagatctcggctgaattctaaaagatctatgtcttgactctatagtaactagagggtgttaaatcggcaacgtcgactctcgagtgagattgttgacggtaccgtattttggatctaggaggaaggatctatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatctgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaactctgaaaggaggaactatatccggatcgagatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaa

SEQ ID NO: 47 - protein, 145 aa, Cupriavidus metallidurans
MNIQIGELAKRTACPVVTIRFYEQEGLLPPPGRSRGNFRLYGEEHVERLQFIRHCRSLDMPLSDVRTLLSYRKRPDQDCGEVNMLLDEHIRQVESRIGALLELKHHLVELREACSGARPAQSCGILQGLSDCVCDTRGTTAHPSD

SEQ ID NO: 48 - DNA, 435 bp, synthetic construct
  source 1..435: mol_type = other DNA
atgaacattcagatcggtgaactggcgaaacgtactgcatgtccagtggtgaccattcgcttttatgagcaagaaggtttgctgccgcccccaggacgttcccgtggtaatttccgcttatacggggaagagcatgttgagcgtttgcaatttatccgccattgccgctcacttgacatgcccttatccgatgtccgcacattgctgtcataccgcaagcgtcctgatcaagattgcggggaggtgaacatgcttctggatgagcatattcgtcaagtagagagtcgtattggcgctctgttagagttaaaacatcacttggtcgagctgcgcgaagcatgctcaggggctcgccctgcacaaagctgcggaatccttcagggcctgagtgattgtgtttgcgatacacgtggcacgacagcacacccctcggat

SEQ ID NO: 49 - DNA, 432 bp, synthetic construct
  source 1..432: mol_type = other DNA
atggaaaataatttggaaaacctgaccattggcgtttttgccaaggcggccggggtcaacgtggagacaatccgcttctatcagcgcaagggcctgttgcgggaaccggacaagccttacggcagcatccgccgctatggggaggcggacgtggttcgggtgaaattcgtgaaatcggcacagcggctggggttcagtctggacgagattgccgagctgttgcggctcgacgatggcacccactgcgaggaggccagcagcctggccgaacacaagctcaaggacgtgcgcgagaagatggccgacttggcgcgcatggaaaccgtgctgtctgaactcgtgtgcgcctgccatgcacgaaaggggaatgtttcctgcccgttgatcgcgtcactacagggcgaagcaggcctggcaaggtcagctatgcct

SEQ ID NO: 50 - protein, 144 aa, synthetic construct
MENNLENLTIGVFAKAAGVNVETIRFYQRKGLLREPDKPYGSIRRYGEADVVRVKFVKSAQRLGFSLDEIAELLRLDDGTHCEEASSLAEHKLKDVREKMADLARMETVLSELVCACHARKGNVSCPLIASLQGEAGLARSAMP

SEQ ID NO: 51 - DNA, 441 bp, synthetic construct
  source 1..441: mol_type = other DNA
atgaaaatcggtgaacttgctaaagccacggactgtgcggttgaaactatccgttactacgagcgtgaacagttgctgccagagcctgcccgttcagacggtaattatcgcttatacacgcaggcacatgtggaacgtctgacgtttattcgcaactgccgtacattggatatgactcttgatgaaatccgttctctgttgcgtttgcgtgattcgccggatgatagctgcgggtccgtgaacgcgttaattgatgaacatattgagcatgtgcaagctcgtatcgatggactggttgcgcttcaggagcaactggtagaacttcgtcgtcgctgcaatgcccaaggtgctgagtgtgccatcttacagcaattggagacgaacggtgctgtttcggtccccgaaacagaacactcccatgtgggtcgctctcacggccac

SEQ ID NO: 52 - protein, 147 aa, synthetic construct
MKIGELAKATDCAVETIRYYEREQLLPEPARSDGNYRLYTQAHVERLTFIRNCRTLDMTLDEIRSLLRLRDSPDDSCGSVNALIDEHIEHVQARIDGLVALQEQLVELRRRCNAQGAECAILQQLETNGAVSVPETEHSHVGRSHGH

(19) United States
(12) Patent Application Publication
(10) Pub. No.: US 2026/0133210 A1
(43) Pub. Date: May 14, 2026

(54) CELL-FREE BIOSENSORS FOR IN VITRO DETECTION OF TARGET MOLECULES

(71) Applicant: Northwestern University, Evanston, IL (US)

(72) Inventors: Holly Mei Ekas, Evanston, IL (US); Brenda M. Wang, Evanston, IL (US); Michael Christopher Jewett, Evanston, IL (US)

(21) Appl. No.: 18/873,316

(22) PCT Filed: Jun. 9, 2023
(86) PCT No.: PCT/US2023/068225

§ 371 Date: Dec. 9, 2024

Related U.S. Application Data
(60) Provisional application No. 63/351,020, filed on Jun. 10, 2022.

(51) Int. Cl. G01N 33/84; G01N 33/53

(52) U.S. Cl. CPC G01N 33/84; G01N 33/5308

(57) ABSTRACT

Disclosed are methods, devices, kits, components, and compositions for detecting a target molecule in a test sample using a cell-free protein synthesis (CEPS) reaction. The methods, devices, kits, components, and compositions may be utilized for detecting target molecules, such as lead, from environmental or biological samples.

### CROSS-REFERENCE TO RELATED APPLICATIONS

This application is the U.S. National Stage entry of PCT/US2023/068225 filed Jun. 9, 2023, which claims the benefit of U.S. Provisional Application 63/351,020 filed Jun. 10, 2022. The above application(s) are incorporated herein by reference in its/their entirety.

### STATEMENT REGARDING FEDERALLY SPONSORED RESEARCH OR DEVELOPMENT

This invention was made with government support under 8J-30009-0029A by the Department of Energy, W52P1J-21-9-3023/CS-21-0701 and FA2386-21-1-4078 by the Department of Defense. The government has certain rights in the invention.

### SEQUENCE LISTING

A Sequence Listing accompanies this application and is submitted as an XML file of the sequence listing named “702581_02359_SequencesList.xml” which is 192,482 bytes in size and was created on Oct. 7, 2025. The sequence listing is electronically submitted via Patent Center with the application and is incorporated herein by reference in its entirety.

### BACKGROUND

The field of the invention relates to cell-free protein synthesis (CFPS) systems. In particular, the field of the invention relates to the use of CFPS systems for in vitro detection of target molecules using cellular extracts.

Cell-free systems offer practical and technical advantages over whole-cell sensors for point-of-use detection of contaminants in aqueous environments like lead, arsenic, mercury, fluoride, and nitrate, and for detecting chemical markers of health and performance in human samples such as blood, urine and saliva. However, the diversity of sensors that can function in E. coli extracts is constrained by the scarcity of characterized strong promoters that can be regulated by allosteric transcription factors. Because engineering promoter strength without affecting inducibility remains an unsolved challenge in synthetic biology, the output signals from cell-free sensors are often undesirably low, particularly when detecting trace contaminants.

To address this problem, here we disclose a platform that utilizes CFPS for in vitro sensing of metabolites including small-molecule metabolites in which the output from a cell-free sensor is amplified using an intermediate RNA polymerase synthesized in situ. Positive feedback introduced through autocatalytic transcription and translation decreases the time required for a generating a detectable signal. By employing orthogonal polymerases in parallel, multiple key target chemicals can be detectable simultaneously in a single reaction vessel. The disclosed technology will have transformative impact toward the engineering of highly sensitive and field-deployable cell-free biosensors for monitoring metabolites and contaminants and may have wide applications including applications for monitoring global water quality.

### SUMMARY

Disclosed are methods, devices, kits, components, and compositions for detecting a target molecule in a test sample using a cell-free protein synthesis (CFPS) reaction. The methods, devices, kits, components, and compositions may be utilized for detecting target molecules which may include small molecules and/or metabolites of small molecules. The components used in the disclosed methods, devices, and kits may be dried or lyophilized and may be present or immobilized on a paper substrate.

The disclosed methods, devices, kits, components, and compositions typically utilize one or more transcription templates that encode and conditionally express one or more exogenous RNA polymerases in the presence of the target molecule. The expressed RNA polymerases in turn induce expression of one or more reporter molecules from transcription templates comprising promoters for the RNA polymerases, thereby amplifying an output signal that is generated in the presence of a detected target molecule.

The disclosed methods may be performed to detect a target molecule in a biological or environmental sample and may include steps of: (i) obtaining a biological or environmental sample which may or may not contain the target molecule and optionally concentrating and/or solubilizing the target molecule in the sample if necessary; and (ii) adding the sample and/or the optionally concentrated and/or solubilized target molecule in the sample to a cell-free protein synthesis (CFPS) reaction, where, if the target molecule is present in the sample, then an output is generated and amplified using an intermediate RNA polymerase synthesized in situ. The disclosed methods utilized positive autocatalytic transcription and translation which decreases the time required for generating a detectable signal.

In some embodiments, the disclosed compositions, kits, systems, or methods include an inhibition scheme to minimize background production, in the absence of the target molecule, of one or more RNA polymerases employed in the compositions, kits, systems, or methods. In some embodiments, the inhibition scheme comprises an inhibitor, optionally wherein the inhibitor is selected from a T7 lysozyme, an RNA or DNA aptamer against T7 RNAP, a DNA mimic of the native T7 RNAP promoter recognition sequence, a sequence-responsive protease that selectively degrades tagged T7 RNAP, and combinations thereof. In some embodiments, the inhibitor comprises a protease, such as basal ClpX protein.

In some embodiments, a composition, kit, or system for detecting a target molecule, such as a chemical compound in a sample is disclosed herein, In some embodiments, the composition, kit, or system comprises one or more of the following components: (a) cellular transcription and translational machinery provided in a lysate from an engineered bacterial strain, or purified separately and reconstituted to defined concentrations, (b) a biosensor molecule that modulates the expression of a target DNA sequence in a DNA transcription template; (c) a DNA transcription template whose expression is configured to be regulated by the biosensor molecule, and encoding a reporter molecule (e.g., a reporter protein or RNA molecule).

In some embodiments of the composition, kit, or system the lysate comprises metabolism from the host strain that provides one or more of the following: (i) energy (e.g., ATP-based regeneration systems or non-phosphate based energy); (ii) cofactor regeneration; (iii) enzymes or transcriptional regulators used for cell-free sensing; or (iv) any combination thereof and optionally exogenously supplied cell-free protein synthesis reagents, including enzyme substrates and cofactors.

In some embodiments of the composition, kit, or system the biosensor molecule is an allosteric transcription factor responsive to the target molecule, wherein the biosensor molecule enables the expression of the reporter molecule in the presence of the target molecule but not in the absence of the target molecule. In some embodiments, the allosteric transcription factor comprises SEQ ID NO: 47 or a variant thereof. In some embodiments, the variant comprises a substitution mutation at one or more of amino acid positions 3, 16, 60, 61, 64, 104, 107, 114, 128, 132. In some embodiments, the variant comprises an alanine substitution mutation at one or more of amino acid positions 3, 16, 60, 61, 64, 104, 107, 114, 128, 132. In some embodiments the variant consists of an alanine substitution mutation at amino acid position 3, 16, 60, 61, 64, 104, 107, 114, 128, 132.

In some embodiments disclosed herein are engineered polypeptide comprising variants of SEQ ID NO: 47, wherein the variants comprise a substitution mutation at one or more of positions 3, 16, 60, 61, 64, 104, 107, 114, 128, 132. In some embodiments, the substation mutation comprises an alanine.

### BRIEF DESCRIPTION OF THE DRAWINGS

FIG. 1A-B. FIG. 1 provides a schematic related to the versatility and robustness of one embodiment of the cell free sensor of the present disclosure. (A) The components of the sensor can be freeze dried and provided in a reaction vessel, such as a microfuge tube. The freeze dried components are stable and can be easily transported. To use, the sensor components can be rehydrated with test substance, e.g., a liquid environmental sample, subject sample, etc. The presence of the target molecule initiates production of a detectable marker which can be detected by the user after a brief incubation. (B) Provides a schematic of one aspect of a detection platform as disclosed herein. An allosteric transcription factor is activated by its ligand (e.g., a metal, protein, small molecule, etc.), initiating transcription of the reporter molecule.

FIG. 2A-C. FIG. 2. (A) Illustrates the typical scheme for cell-free sensing. A sensor plasmid encodes an allosteric transcription factor and a second plasmid, a reporter plasmid, expresses a fluorescent report such as Green Fluorescent Protein (GFP), with the cognate promoter/operator sequence. (B) Illustrates the typical response function of a cell-free sensor where the regulated promoter drives expression of the reporter molecule. (C) Illustrates the goal response function using a cascaded sensor embodiment to enhance sensitivity of the system. In this embodiment, the regulated promoter drives expression of T7 RNA polymerase (RNAP) or a variant of T7 RNAP, and T7 RNAP then drives the expression of a reporter molecule from a corresponding promoter.

FIG. 3A-C. FIG. 3 shows three different platforms for the biosensor system of the present disclosure. (A) Shows a platform comprising the expression of a reporter or signal molecule (e.g., GFP), in response to the target molecule activating its transcription factor and stimulating the promoter (e.g., the E. coli J23119 promoter) to transcribe the reporter molecule. In this platform the components of the sensor include transcription and translation components. (B) In the cascaded sensor embodiment, the regulated promoter drives expression of T7 RNA polymerase (RNAP) or a variant of T7 RNAP, and T7 RNAP then drives expression of the reporter molecule from a corresponding promoter. Again, the system includes transcription and translation components. (C) Illustrates a third embodiment of the sensor systems disclosed herein, utilizing signal amplification and positive feedback and termed “double cascade.” In this embodiment, T7 RNAP is made through the top, regulated layer of the cascade and is able to amplify itself autocatalytically. The top level of this cascade is termed the “source,” the mid-level is termed the “transducer” or “amplifier” and the third level is termed the “reporter.” In some embodiments, the DNA templates for one or more components of this system is prepared in vitro by, for example, isothermal assembly and the polymerase chain reaction (PCR).

FIG. 4A-D. FIG. 4 illustrates various aspects of the biosensors of the present disclosure. (A) The “enriched” extract that contains the allosteric transcription factor can be mixed against a “blank” unenriched extract to modulate the concentration of the transcription factor in the reaction. (B) For tighter control and specificity of a cascaded system, if T7 RNA polymerase is used to drive expression of the allosteric transcription factor in the host strain of extract, an engineered variant of T7 RNAP may be used as the output of the regulated promoter. Exemplary T7 RNAP mutants are illustrated. (C) Shows the kinetics of a cascade amplifier. The E. coli RNAP is used to express four T7 RNAP variants from a mock sensor plasmid (5 nM) that contains the consensus E. coli promoter J23119. The corresponding reporter plasmid is added at 5 nM. The kinetics of T7 RNAP synthesis lead to a time delay of about 20-30 minutes relative to a reaction that uses purified WT T7 RNAP. Reaction conditions were as follows: triplicate 10 μL technical replicates for cell-free gene expression reaction at 30° C. (D) Orthogonality of T7 variants to the wild-type T7 RNAP. 5 nM of each orthogonal T7 RNAP reporter plasmid was supplied to a cell-free reaction in the presence of the WT T7 RNAP. The leak is lowest with the AKSIRV promoter. Reporter yields are from triplicates of four-hour sfGFP yields on a plate reader at 30° C. All cell-free reactions were prepared as previously described with the following composition: 30 v/v % total S12 extract prepared from the E. coli strain BL21 Star (DE3), grown to optical density 3.0 sonicated, and processed by ribosomal runoff reaction and dialysis; 8 mM magnesium glutamate, 10 mM ammonium glutamate, and 60 mM potassium glutamate; 1.2 mM ATP; 825 μM of CTP, GTP, and UTP; 34 mg/L folinic acid; 171 mg/L tRNA; 2.5 mM each amino acid; 30 mM phosphoenolpyruvate (PEP); 330 μM nicotinamide adenine dinucleotide (NAD); 270 μM coenzyme A; 4 mM potassium oxalate; 1 mM putrescine; 1.5 mM spermidine; 57 mM HEPES; midiprepped plasmid DNA to the requisite concentration; and the remainder water.

FIG. 5A-D. Experimental transcription, translation, and resource limitation kinetic parameters validate cascade models. (A)-(C) Parameterization of the kinetics of transcription and translation in the cell-free sensor. We assume a model of transcription and translation under finite resources, accounting for utilization of RNAPs and ribosomes as well as an exponential decay in transcription and translation rates caused by byproduct accumulation. (D) This model is backed up by experimental data where we simultaneously measure RNA and protein levels using a version of sfGFP that is tagged at the 3′ end with the sequence of the malachite green RNA aptamer. Experimental data are 4-hour endpoint reads measured in triplicate from a cell-free gene expression reaction supplied with 33% PhlF-containing extract by volume and 5 nM reporter plasmid. Reaction conditions were as follows: triplicate 10 μL technical replicates for cell-free gene expression reaction at 30° C. for four hours.

FIG. 6A-B. FIG. 6 shows that cascades are predicted to improve the dose response more than noncascaded physiochemical optimizations, both for ON state (for most promoters) and Limit of Detection. Model prediction of the improved dose response behavior using a cascaded amplifier (blue) relative to the no-amplifier condition (black), with a strong bacterial promoter and low transcriptional leak. The cascade improves the dose response far more than can be achieved by tuning DNA concentration in the absence of the cascade. (A) Absolute signal of sfGFP using parameterized data in a 4-hour cell-free gene expression experiment. (B) Signal normalized between the minimum and maximum fluorescence

FIG. 7A-G. Development of a panel of uncascaded cell-free sensors that detect inorganic metabolites: (A) arsenic, (B) mercury, (C), (D) nitrate, (E) copper, (F) lead, and (G) cadmium. Optimization of the ratio of extract enriched with the relevant transcription factor (or sensor kinase and response regulator for the nitrate two-component system), with the balance of the extract ratio provided by a blank extract from BL21*(DE3) E. coli. The optimal extract ratio (measured by the activation ratio, ON/OFF at saturating analyte concentration) is bolded on each plot. Reaction conditions were as follows: triplicate 10 μL technical replicates for cell-free gene expression reaction at 30° C. for four hours. The reporter plasmid was supplied at 20 nM in each case. The data are background-subtracted from a no-DNA control.

FIG. 8A-G. Development of a panel of cascaded cell-free sensors that detect inorganic metabolites: (A) arsenic, (B) mercury, (C) nitrate, (D) copper, (E) lead, (F) fluoride, and (G) cadmium. Optimization of the sensor plasmid (regulated promoter+T7 AKSIRV RNAP) concentration with 5 nM AKSIRV reporter plasmid in each case. The optimal concentration (measured by the activation ratio, ON/OFF at saturating analyte concentration) is bolded on each plot. Reaction conditions were as follows: triplicate 10 μL technical replicates for cell-free gene expression reaction at 30° C. for four hours. The reporter plasmid was supplied at 5 nM in each case and the data are background-subtracted from a no-DNA control.

FIG. 9A-G. Comparative dose responses for a panel of cascaded cell-free sensors that detect inorganic metabolites: (A) mercury, (B) copper, (C) lead, (D) cadmium, (E) arsenic, (F) fluoride, and (G) nitrate, black represents the optimized dose response curve for the noncascaded sensor (measured from experimental triplicate and normalized to a FITC standard after 4-hour reaction at 30° C.) and blue represents the optimized dose response curve for the cascaded sensor. In each case, the cascade improves the response function (increasing signal and/or shifting the curve to the left indicating enhancement of limit of detection). Dashed vertical line represents either the WHO legal limit (or, for mercury, the EPA limit, which is more stringent) in drinking water. Reaction conditions were as follows: triplicate 10 μL technical replicates for cell-free gene expression reaction at 30° C. for four hours. The reporter plasmid is supplied at 5 nM (cascade) or 20 nM (noncascaded) and the concentration of the cascaded sensor plasmid is the optimal concentration from FIG. 8.

FIG. 10A-G. Shows the same data as FIG. 9 but with the data re-normalized to have “fraction of maximum fluorescence”, with normalization error propagated. (A) arsenic, (B) fluoride, (C) mercury, (D) cadmium, (E) copper, (F) lead, and (G) nitrate.

FIG. 11. Shows results of a cascaded system detecting Hg at the legal limit from a freeze-dried sensor components. Kinetics of activation of a freeze-dried cell-free mercury sensor at the WHO legal limit (6 ppb). This represents the best dynamic range for a cell-free mercury sensor that has a fluorescent protein output in the literature at this limit. The freeze-dried sensor was prepared following the same physiochemical reaction conditions as before, prepared to 33 μL scale, then lyophilized at 0.04 mbar and −80° C. overnight. The reactions were rehydrated with either water or 6 ppb HgCl₂ in water and incubated at 30° C. for eight hours.

FIG. 12A-B. FIG. 12 illustrates an autocatalytic amplification, double-cascade system. (A) is the same as FIG. 3C and illustrates a third embodiment of the sensor systems disclosed herein, utilizing signal amplification and positive feedback and termed “double cascade.” In this embodiment, T7 RNAP is made through the top, regulated layer of the cascade and is able to amplify itself autocatalytically. In some embodiments, the DNA templates for one or more components of this system is prepared in vitro through, for example, isothermal assembly and the polymerase chain reaction (PCR). (B) shows a predicted dose response behavior through the implementation of an autocatalytic cascade, in a system with a low transcription leak, shifting the effective response another ˜order of magnitude to the left.

FIG. 13A-B. Proof of concept of a double-cascade amplifier which is not autocatalytic. (A) In this example, Hg-inducible expression of one variant of T7 RNAP leads to expression of a second, orthogonal T7 RNAP through a linear expression template. (B) At the WHO legal limit (30 nM=6 ppb), the double cascaded variant (fifth set of bars) improves the ON signal compared to either the uncascaded version (first set of bars) or either of the single cascades (second, third, and sixth set of bars). The fourth set of bars is a double cascade control, where the intermediate amplifier plasmid does not generate any additional polymerase that binds to the reporter. pMer: promoter that recognizes the allosteric transcription factors MerR which is activated by mercury; GFP: Green Fluorescent Protein; AKSIRV: T7 polymerase that bind the T7 promoter mutant pAKSIRV (TAATACCTGACACTATAGG; SEQ ID NO:3); pAKSIRV: promoter mutant for the AKSIRV polymerase; RV: polymerase that binds the T7 promoter mutant pRV (TAATAACCCTCACTATAGG; SEQ ID NO:2); pRV: promoter mutant for the RV polymerase; sfGFP: super-folded Green Fluorescent Protein. Reactions were performed as follows. triplicate 10 μL technical replicates for cell-free gene expression reaction at 30° C. for four hours.

FIG. 14. Shows the results of optimization of double cascaded amplifier. As predicted from the resource-constrained model, optimal sensor response will occur at a small but finite concentration of the intermediate node of the cascade. Pictured are experimental data: when provided a small amount of AKSIRV promoter expressed under the strong constitutive bacterial promoter J23119 (sequence: TTGACAGCTAGCTCAGTCCTAGGTATAATACTAGT; SEQ ID NO:6) (0.1 nM) the double cascade amplifies the low signal only when the transducer is at around 0.5 nM. Reactions were provided with GamS (a nuclease inhibitor) to protect the linear expression templates. P_{reg}: J23119 promoter driven by endogenous E. coli RNA polymerase and driving expression of the AKSIRV T7 polymerase mutant (i.e., the T7 RNA polymerase that binds the AKSIRV mutant promoter, pAKSIRV). Reactions were performed according to the same molecular compositions at 10 μL technical duplicates for cell-free gene expression reaction at 30° C. for four hours. RV: T7 RV mutant polymerase (i.e., the T7 RNA polymerase that binds the RV mutant promoter, pRV); sfGFP: super-folded Green Fluorescent Protein. In the graph, the first bar, “AKSIRV source, AKSIRV reporter” indicates that there was only a single plasmid in this system: AKSIRV polymerase was produced by a first plasmid, comprising an E. coli J23119 promoter and activated by endogenous E. coli RNA polymerase—the base case to be amplified. The next two bars, “no source, transducer, RV reporter” are a set of experimental controls and demonstrate that the transducer can leak at high concentration due to the production of RV polymerase that can drive reporter expression. The next 7 bars “AKSIRV source, transducer, RV reporter” are a titration of the transducer and demonstrate that as this construct's concentration increases, production of RV polymerase through the cascade leads to amplification of signal and resource limitations at high transducer concentration.

FIG. 15. Proof-of-concept for autocatalytic amplification. The presence of a linear expression template (LET) allowing for AKSIRV autocatalytic amplification improves the kinetics and final yield of sfGFP for an unregulated sensor. The sensor reaction was prepared as previously described in technical triplicates at 10 μL scale and the reaction was run at 30° C. for four hours.

FIG. 16. Proof-of-concept for autocatalytic cascaded sensing at 10 nM HgCl₂ (the most stringent limit). Implementing an AKSIRV autocatalytic cascade improves the signal to a visible threshold (>1 μM FITC) without greatly increasing the leak, when compared against the single AKSIRV cascade. Sensor reaction conditions were as follows: technical triplicates at 10 μL scale at 30° C. for four hours.

FIG. 17. Kinetics of autocatalytic amplification. Even in the absence of a source of AKSIRV, an AKSIRV autocatalytic amplifier turns ON to high signal at very low concentrations of its linear expression template (LET), indicating that tuning will likely be necessary to ensure robustness. Reaction conditions were as follows: technical duplicates at 10 μL scale at 30° C. for four hours.

FIG. 18A-G. Figures A-C show that orthogonal T7 RNA polymerase variants can enable one-pot sensor multiplexing. In this multiplex embodiment, the transcription factors MerR, AsR, NarX, NarL are pre-enriched in the extract(s) to sense (A) Hg, (B) As, and (C) nitrate. Figures (D)-(G) show an alternative platform for multiplexing cell-free outputs using the BioBits color palette. In (D) and (E), the first line is blue, the second is green (Pmer), and the third line is red (Pasr). Reactions shown in FIG. 18G were prepared as follows. Technical triplicates at 10 μL scale at 30° C. for four hours.

FIG. 19A-B. De-sensitizing using tunable proteolysis. (A) Model for stoichiometric inhibition. A programmable protease (mf-lon) that targets only tagged proteins (in this case, the orthogonal T7 RNAP variant that is the output of the sensor) is included in the reaction and degrades is target with zeroth order kinetics. (B) Predicted dose response behavior. An inhibitor is expected to shift a DR curve down and to the right, with the goal of mitigating sensor leak.

FIG. 20A-C. Overexpressed mf-Lon, pdt tag, and ATP contribute to protein degradation. Proof of concept for stoichiometric inhibition using mf-Lon. An mf-Lon enriched extract was mixed with a cellular extract containing pdt-tagged sfGFP. Increasing the concentration of mf-Lon and supplying additional ATP (a co-substrate for the reaction) results in some signal decay. (A) 0% mf-Lon; (B) 10% mf-Lon; (C) 50% mf-Lon. Reactions were performed as follows: mf-Lon enriched cellular extract from a BL21 Star (DE3) strain was directly mixed with 50% cellular extract from a BL21 Star (DE3) strain overexpressing pdt-tagged sfGFP, varying the ratio of the two extracts and making up the additional volume with a blank extract, and supplying exogenous ATP. The additional reaction components (e.g., salts and buffers) were left out.

FIG. 21. Design of mitigating cross-talk of sensors. Experimental measurement of crosstalk between four metal-sensing aTFs using cell-free response. Heatmap is colored more brightly to indicate stronger fluorescent signal. In this example, there is crosstalk for the lead sensor with cadmium, indicating that this strategy will be necessary to distinguish the two analytes.

FIG. 22A-E. Alternative options for reducing background in biosensors. (A) A “T7 lysozyme-enriched” extract dose not inhibit T7 RNAP. An alternative embodiment would be to expression of the lysozyme from the J23119 promoter in vitro. (B) An anti-T7 aptamer expressed in situ does not inhibit T7 RNAP. An alternative embodiment includes purifying the aptamer from sp6 RNAP and providing the aptamer to the biosensor reaction mixture at high concentrations. (C) A T7 promoter mimic may selectively inhibit low concentrations of wild-type T7 RNAP. An alternative embodiment includes higher concentrations of promoter and measurement of T7 dose responses. (D) While SsrA mediated degradation in basal CplX may be too potent, it can reduce leak. (E) Provides a schematic showing the implementation of protein level logic to address sensor promiscuity. Experiments were carried out in experimental technical replicates (N=2, generally) at 30° C. for 4 hours.

FIG. 23. Is a schematic depiction comparing various methods of water diagnostics.

FIG. 24. Schematically depicts various features of an allosteric transcription factor used as a biosensor.

FIG. 25. Three-dimensional structures of metalloregulating transcription factors: MerR, which senses mercury, CadR, which senses cadmium, and PbrR, which senses lead, and provides a schematic of how an allosteric transcription factor can functions in the compositions, kits, systems, and methods disclosed herein.

FIG. 26. Is a schematic depiction of an in vitro cell-free assay for screening allosteric transcription factor promiscuity as described herein in Example 2.

FIGS. 27, 28, and 29. Provide exemplary data with respect to PbrR variants and wild type PbrR expressed in vitro with a fluorescent reporter.

FIG. 30. Depicts a workflow for alanine scanning of PbrR to probe the sequence-function relationship as described in Example 2.

FIG. 31. Depicts the results of the initial alanine screening described with respect to FIG. 30.

FIGS. 32 and 33. Depict the 50 hotspots chosen from the alanine screening described with reference to FIGS. 30 and 31 and in Example 2.

FIG. 34. Depicts the results of testing a site saturation mutagenesis (SSM) library for the hotspots identified in FIGS. 32 and 33 as described in Example 2.

FIGS. 35 and 36. Depict the hand-validated results of the SSM library screen described with reference to FIG. 34 and in Example 2.

FIG. 37. Depicts the 76 combination variants that were made and tested for their ability to induce expression of the reporter as described in Example 2.

FIG. 38. Depicts the tests results of the combination variants that have greater sensitivity than the individual mutations that they are comprised of as described in Example 2.

FIG. 39. Depicts test results for expression of the reporter of variants with increased sensitivity to lead as described in Example 2.

FIGS. 40 and 41. Depicts the use of enriched extracts with certain PbrR variants and that the enriched extract sensors are as sensitive as the linear expression template (LET) sensors as described in Example 2.

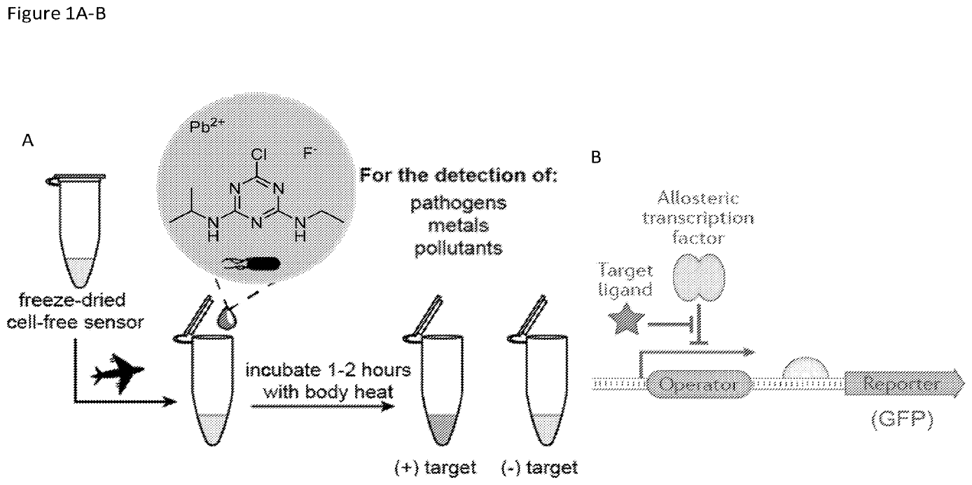

### DETAILED DESCRIPTION

The presently disclosed subject matter is described herein using several definitions, as set forth below and throughout the application.

### Definitions

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

Unless otherwise specified or indicated by context, the terms “a”, “an”, and “the” mean “one or more.” For example, “a component,” “a metabolite,” and “a contaminant,” should be interpreted to mean “one or more components,” “one or more metabolites,” and “one or more contaminants,” respectively. For example, “a composition,” “a system,” “a kit,” “a method,” “a protein,” “a vector,” “a domain,” “a binding site,” and “an RNA” should be interpreted to mean “one or more compositions,” “one or more systems,” “one or more kits,” “one or more methods,” “one or more proteins,” “one or more vectors,” “one or more domains,” “one or more binding sites,” and “one or more RNAs,” respectively.

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

Ranges recited herein include the defined boundary numerical values as well as sub-ranges encompassing any non-recited numerical values within the recited range. For example, a range from about 0.01 mM to about 10.0 mM includes both 0.01 mM and 10.0 mM. Non-recited numerical values within this exemplary recited range also contemplated include, for example, 0.05 mM, 0.10 mM, 0.20 mM, 0.51 mM, 1.0 mM, 1.75 mM, 2.5 mM 5.0 mM, 6.0 mM, 7.5 mM, 8.0 mM, 9.0 mM, and 9.9 mM, among others. Exemplary sub-ranges within this exemplary range include from about 0.01 mM to about 5.0 mM; from about 0.1 mM to about 2.5 mM; and from about 2.0 mM to about 6.0 mM, among others.

As used herein, the terms “regulation” and “modulation” may be utilized interchangeably and may include “promotion” and “induction.” For example, a transcription factor that regulates or modulates expression of a target gene may promote and/or induce expression of the target gene. In addition, the terms “regulation” and “modulation” may be utilized interchangeably and may include “inhibition” and “reduction.” For example, a transcription factor that regulates or modulates expression of a target gene may inhibit and/or reduce expression of the target gene.

As used herein, the term “sample” may include “biological samples” and “non-biological samples.” Biological samples may include samples obtained from a human or non-human subject. Biological samples may include but are not limited to, blood samples and blood product samples (e.g., serum or plasma), urine samples, saliva samples, fecal samples, perspiration samples, and tissue samples. Non-biological samples may include but are not limited to aqueous samples (e.g., watershed samples) and surface swab samples.

The term “target molecule” means any molecule of interest in a test sample and may include so-called “small molecules” or metabolites of small molecules. Target molecules may be referred to herein alternatively as “analytes,” “metabolites,” and “contaminants.” Exemplary target molecules include metabolites, chemical compounds, and nucleic acids. By way of example, but not by way of limitation, target molecules include phloroglucinol, mercury, arsenic or its oxides, nitrate, fluoride, cyanuric acid, lead, copper, zinc, chromium or its oxides, or atrazine.

The term “metabolite” means a molecule to which a target molecule is converted, for example, by one or more components such as enzymes that are present in a cell-free protein synthesis (CFPS) reaction mixture and/or that are added to a CFPS reaction mixture.

The term “transcription factor” refers to a protein that regulates transcription of another protein, typically by interacting by one or more cis-acting DNA sequence in or near the promoter for the other protein. A transcription factor may increase expression or decrease expression depending upon whether the transcription factor is activated or deactivated. A transcription factor may become activated or deactivated by an interaction with another molecule (e.g., a metabolite as described above). Such transcription factors are termed allosteric transcription factors.

The term “reporter molecule” refers to a molecule (e.g., a reporter protein or RNA) that can be detected in a reaction mixture, such as a CFPS reaction mixture, typically in response to the presence of a target molecule or a metabolite thereof being present in the reaction mixture. For example, a reporter molecule may be expressed and detected in a CFPS reaction mixture when a target molecule or a metabolite thereof activates a transcription factor which promotes expression of the reporter protein in the CFPS reaction mixture. Exemplary reporter molecules include fluorescent molecules, such as Green Fluorescent Protein and super-folded Green Fluorescent Protein. Any number of reporter molecules well known in the art (Yellow, Blue, and Red Fluorescent Proteins, mCherry, etc.) can be used in the methods, systems, compositions, and kits of the present disclosure.

The term “promoter” refers to a cis-acting DNA sequence that directs RNA polymerase and other trans-acting transcription factors to initiate RNA transcription from the DNA template that includes the cis-acting DNA sequence.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, bacteriophage polymerases such as, but not limited to, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

As used herein, “translation template” refers to an RNA product of transcription from an expression template that can be used by ribosomes to synthesize polypeptide or protein.

As used herein, coupled transcription/translation (“Tx/Tl”), refers to the de novo synthesis of both RNA and a sequence defined biopolymer from the same extract. For example, coupled transcription/translation of a given sequence defined biopolymer can arise in an extract containing an expression template and a polymerase capable of generating a translation template from the expression template. Coupled transcription/translation can occur using a cognate expression template and polymerase from the organism used to prepare the extract. Coupled transcription/translation can also occur using exogenously-supplied expression template and polymerase from an orthogonal host organism different from the organism used to prepare the extract. In the case of an extract prepared from a yeast organism, an example of an exogenously-supplied expression template includes a translational open reading frame operably coupled a bacteriophage polymerase-specific promoter and an example of the polymerase from an orthogonal host organism includes the corresponding bacteriophage polymerase.

### Polynucleotides and Uses Thereof

The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

The terms “nucleic acid” and “oligonucleotide,” as used herein, may refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The terms “target,” “target sequence,” “target region,” and “target nucleic acid,” as used herein, are synonymous and may refer to a region or sequence of a nucleic acid which is to be hybridized and/or bound by another nucleic acid.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)ₙ sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, RNA polymerases of bacteriophages (e.g. T3 RNA polymerase, T7 RNA polymerase, SP6 RNA polymerase, Syn5 RNA polymerase), and E. coli RNA polymerase, among others. The foregoing examples of RNA polymerases are also known as DNA-dependent RNA polymerase. The polymerase activity of any of the above enzymes can be determined by means well known in the art.

Also contemplated for us in the disclosed compositions, systems, kits, and methods are engineered RNA polymerase. For example, an engineered polymerase may be a non-naturally occurring RNA polymerase whose amino acid sequence has been engineered to include one or more of an insertion, a deletion, or a substitution relative to the amino acid sequence of a naturally occurring or wild-type RNA polymerase.

The term “promoter” refers to a cis-acting DNA sequence that directs RNA polymerase and other trans-acting transcription factors to initiate RNA transcription from the DNA template that includes the cis-acting DNA sequence.

As used herein, “an engineered transcription template” or “an engineered expression template” refers to a non-naturally occurring nucleic acid that serves as substrate for transcribing at least one RNA. As used herein, “expression template” and “transcription template” have the same meaning and are used interchangeably. Engineered include nucleic acids composed of DNA or RNA. Suitable sources of DNA for use in a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms.

“Transformation” or “transfection” describes a process by which exogenous nucleic acid (e.g., DNA or RNA) is introduced into a recipient cell. Transformation or transfection may occur under natural or artificial conditions according to various methods well known in the art, and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam.™. and Lipofectin.™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time.

The polynucleotide sequences contemplated herein may be present in expression vectors. For example, the vectors may comprise a polynucleotide encoding an ORF of a protein operably linked to a promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into mRNA or another RNA transcript) and/or the process by which a transcribed mRNA is subsequently translated into peptides, polypeptides, or proteins. Transcripts and encoded polypeptides may be collectively referred to as “gene product.”

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refer to a recombinant nucleic acid that has been engineered to express a heterologous polypeptide (e.g., the fusion proteins disclosed herein). The recombinant nucleic acid typically includes cis-acting elements for expression of the heterologous polypeptide.

In the methods contemplated herein, a host cell may be transiently or non-transiently transfected (i.e., stably transfected) with one or more vectors described herein. A cell transfected with one or more vectors described herein may be used to establish a new cell line comprising one or more vector-derived sequences. In the methods contemplated herein, a cell may be transiently transfected with the components of a system as described herein (such as by transient transfection of one or more vectors), and modified through the activity of a complex, in order to establish a new cell line comprising cells containing the modification but lacking any other exogenous sequence.

### Peptides, Polypeptides, and Proteins

As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation), hydroxylation, iodination (e.g., of thyroid hormones), and phosphorylation (e.g., the addition of a phosphate group, usually to serine, tyrosine, threonine or histidine).

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may have one or more insertions, deletions, or substitution of at least one amino acid residue relative to a reference polypeptide.

Regarding proteins, a “deletion” refers to a change in the amino acid sequence that results in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide). A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full-length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

Regarding proteins, the phrases “percent identity” and “% identity,” refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. The following table provides a list of exemplary conservative amino acid substitutions which are contemplated herein:

| Original | |
|---|---|
| Residue | Conservative Substitution |
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acids typically disrupt (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, mutants, variants, or described herein may have one or more functional or biological activities exhibited by a reference polypeptide (e.g., one or more functional or biological activities exhibited by wild-type protein).

In some embodiments of the disclosed compositions, systems, kits, and methods, the components may be substantially isolated or purified. The term “substantially isolated or purified” refers to components that are removed from their natural environment, and are at least 60% free, preferably at least 75% free, and more preferably at least 90% free, even more preferably at least 95% free from other components with which they are naturally associated.

### Cell-Free Protein Synthesis (CFPS)

The disclosed subject matter relates in part to methods, devices, kits and components for cell-free protein synthesis. Cell-free protein synthesis (CFPS) is known and has been described in the art. (See, e.g., U.S. Pat. Nos. 6,548,276; 7,186,525; 8,734,856; 7,235,382; 7,273,615; 7,008,651; 6,994,986 U.S. Pat. Nos. 7,312,049; 7,776,535; 7,817,794; 8,298,759; 8,715,958; 9,005,920; U.S. Publication No. 2014/0349353, U.S. Publication No. 2016/0060301, U.S. Publication No. 2018/0016612, and U.S. Publication No. 2018/0016614, the contents of which are incorporated herein by reference in their entireties). A “CFPS reaction mixture” typically contains a crude or partially-purified bacterial extract (as used herein the terms “extract” and “lysate” are used interchangeably), an RNA translation template, and a suitable reaction buffer for promoting cell-free protein synthesis from the RNA translation template. In some aspects, the CFPS reaction mixture can include exogenous RNA translation template. In other aspects, the CFPS reaction mixture can include a DNA expression template encoding an open reading frame operably linked to a promoter element for a DNA-dependent RNA polymerase. In these other aspects, the CFPS reaction mixture can also include a DNA-dependent RNA polymerase to direct transcription of an RNA translation template encoding the open reading frame. In these other aspects, additional NTP's and divalent cation cofactor can be included in the CFPS reaction mixture. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the invention. For example, the cellular transcription and translational machinery may be provided in a lysate from an engineered bacterial strain, or the transcription and translational machinery may be purified separately and reconstituted to defined concentrations. In some embodiments, a lysate may be from an engineered bacterial strain, and include cellular transcriptional and translational machinery, and may also include other as other cellular proteins.

The disclosed cell-free protein synthesis systems may utilize components that are crude and/or that are at least partially isolated and/or purified. As used herein, the term “crude” may mean components obtained by disrupting and lysing cells and, at best, minimally purifying the crude components from the disrupted and lysed cells, for example by centrifuging the disrupted and lysed cells and collecting the crude components from the supernatant and/or pellet after centrifugation. The term “isolated or purified” refers to components that are removed from their natural environment, and are at least 60% free, preferably at least 75% free, and more preferably at least 90% free, even more preferably at least 95% free from other components with which they are naturally associated.

An aspect of the invention is a platform for preparing a sequence defined protein in vitro which may be utilized for detecting a target molecule or metabolite thereof. The platform for preparing a sequence defined polymer or protein in vitro comprises a cellular extract from a host strain. Because CFPS exploits an ensemble of catalytic proteins prepared from the crude lysate of cells, the cell extract (whose composition is sensitive to growth media, lysis method, and processing conditions) is the most critical component of extract-based CFPS reactions. A variety of methods exist for preparing an extract competent for cell-free protein synthesis, including U.S. patent application Ser. No. 14/213,390 to Michael C. Jewett et al., entitled METHODS FOR CELL-FREE PROTEIN SYNTHESIS, filed Mar. 14, 2014, and now published as U.S. Patent Application Publication No. 2014/0295492 on Oct. 2, 2014, and U.S. patent application Ser. No. 14/840,249 to Michael C. Jewett et al., entitled METHODS FOR IMPROVED IN VITRO PROTEIN SYNTHESIS WITH PROTEINS CONTAINING NON STANDARD AMINO ACIDS, filed Aug. 31, 2015, and now published as U.S. Patent Application Publication No. 2016/0060301, on Mar. 3, 2016, the contents of which are incorporated by reference.

The platform may comprise an expression template, a translation template, or both an expression template and a translation template. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. In certain embodiments the platform comprises both the expression template and the translation template. In certain specific embodiments, the platform may be a coupled transcription/translation (“Tx/Tl”) system where synthesis of translation template and a sequence defined biopolymer from the same cellular extract.

The platform may comprise one or more polymerases capable of generating a translation template from an expression template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract.

Altering the physicochemical environment of the CFPS reaction to better mimic the cytoplasm can improve protein synthesis activity. The following parameters can be considered alone or in combination with one or more other components to improve robust CFPS reaction platforms based upon crude cellular extracts (for examples, S12, S30 and S60 extracts).

The temperature may be any temperature suitable for CFPS. Temperature may be in the general range from about 10° C. to about 40° C., including intermediate specific ranges within this general range, include from about 15° C. to about 35° C., from about 15° C. to about 30° C., form about 15° C. to about 25° C. In certain aspects, the reaction temperature can be about 15° C. about 16° C., about 17° C., about 18° C., about 19° C., about 20° C., about 21° C., about 22° C., about 23° C., about 24° C., about 25° C.

The CFPS reaction can include any organic anion suitable for CFPS. In certain aspects, the organic anions can be glutamate, acetate, among others. In certain aspects, the concentration for the organic anions is independently in the general range from about 0 mM to about 200 mM, including intermediate specific values within this general range, such as about 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM and about 200 mM, among others.

The CFPS reaction can also include any halide anion suitable for CFPS. In certain aspects the halide anion can be chloride, bromide, iodide, among others. A preferred halide anion is chloride. Generally, the concentration of halide anions, if present in the reaction, is within the general range from about 0 mM to about 200 mM, including intermediate specific values within this general range, such as those disclosed for organic anions generally herein.

The CFPS reaction may also include any organic cation suitable for CFPS. In certain aspects, the organic cation can be a polyamine, such as spermidine or putrescine, among others. Preferably polyamines are present in the CFPS reaction. In certain aspects, the concentration of organic cations in the reaction can be in the general about 0 mM to about 3 mM, about 0.5 mM to about 2.5 mM, about 1 mM to about 2 mM. In certain aspects, more than one organic cation can be present.

The CFPS reaction can include any inorganic cation suitable for CFPS. For example, suitable inorganic cations can include monovalent cations, such as sodium, potassium, lithium, among others; and divalent cations, such as magnesium, calcium, manganese, among others. In certain aspects, the inorganic cation is magnesium. In such aspects, the magnesium concentration can be within the general range from about 1 mM to about 50 mM, including intermediate specific values within this general range, such as about 1 mM, about 2 mM, about 3 mM, about 5 mM, about 6 mM, about 7 mM, about 8 mM, about 9 mM, about 10 mM, among others. In preferred aspects, the concentration of inorganic cations can be within the specific range from about 4 mM to about 9 mM and more preferably, within the range from about 5 mM to about 7 mM.

The CFPS reaction includes NTPs. In certain aspects, the reaction use ATP, GTP, CTP, and UTP. In certain aspects, the concentration of individual NTPs is within the range from about 0.1 mM to about 2 mM.

The CFPS reaction can also include any alcohol suitable for CFPS. In certain aspects, the alcohol may be a polyol, and more specifically glycerol. In certain aspects the alcohol is between the general range from about 0% (v/v) to about 25% (v/v), including specific intermediate values of about 5% (v/v), about 10% (v/v) and about 15% (v/v), and about 20% (v/v), among others.

### Biosensors: Compositions, Kits, and Systems

The technology described herein relates generally to microbial-based biosensor compositions, systems, and kits for the detection of small molecules and analytes (e.g., metabolites, chemical compounds, nucleic acids), based on an analyte-responsive transcription factor-DNA binding mechanism, resulting in the expression of a detectable reporter protein. The biosensors employ one or more analyte-responsive transcription factor-DNA binding platforms for the cell free detection of target molecules. In some embodiments, the biosensor systems include one or more signal amplifiers to provide a cascade of polymerase expression, and/or include one or more inhibitors to decrease background (increase signal to noise ratio) of the reporter protein.

As used herein, the term “biosensor” refers to a reaction mixture comprising all of the components necessary to detect an analyte of interest. In some embodiments, the biosensor is provided as freeze-dried components contained in a vessel, such as a microfuge tube, test tube, or a multi-well plate. Upon rehydration of the components with a liquid or liquefied sample, the analyte of interest, if present, initiates a reaction in the vessel resulting in the expression of a reporter molecule.

The biosensor is designed to detect a ligand (the analyte) of an allosteric transcription factor. Once the ligand binds its transcription factor, a cascade of transcription and translation events occurs. Accordingly, the biosensors comprise the components for transcription and translation reactions and include the necessary enzymes, co-factors, nucleotides, amino acids, energy source, etc. These components can be provided individually, or can be provided as one or more extracts for CFPS as described above.

By way of example, a biosensor of the present disclosure comprises one or more lysates from engineered bacterial strains, the lysate comprising cellular transcriptional and translational machinery, and optionally other cellular proteins, co-factors, energy sources (e.g., ATP-based cellular energy or non-phosphate based energy); a biosensor molecule that modulates the expression of a target DNA sequence in a DNA transcription template (e.g., an ATF); a DNA transcription template whose expression is configured to be regulated by the biosensor molecule, and encoding the expression of additional RNA polymerase not present in the lysate (e.g., an exogenous or orthogonal RNA polymerase); and a second DNA transcription template encoding the expression of a reporter molecule (e.g., a reporter protein or RNA molecule) whose transcription is controlled by the expressed additional RNA polymerase (e.g., wherein the second DNA transcription template comprises a promoter for the exogenous or orthogonal RNA polymerase).

The biosensors comprise at least one biosensor molecule, such as an allosteric transcription factor (ATF). The term “allosteric transcription factor” as used herein refers to regulatory proteins that contain a DNA-binding domain as well as a ligand-binding domain that is able to recognize small molecules with high specificity and selectivity. In the presence of a target small molecule (i.e., the transcription factor ligand), transcription factor affinity for its DNA binding sequence is modulated, facilitating the repressor or derepressor regulation of downstream gene expression. In some embodiments, the biosensors disclosed herein comprise a plasmid (e.g., a sensor plasmid) that expresses the ATF either in the biosensor (e.g., transcription and translation of the ATF occurs upon rehydration of the biosensor components by adding liquid the sample), or in the host strain used for making the biosensor (e.g., as a component of a CFPS reaction). For example, in some embodiments, the biosensors comprise the ATF protein, and the biosensor molecule is overexpressed in the host strain prior to making the extract for cell-free protein synthesis. Additionally or alternatively, the biosensor molecule (e.g., an ATF) comprises an isolated protein and is added to the biosensor components.

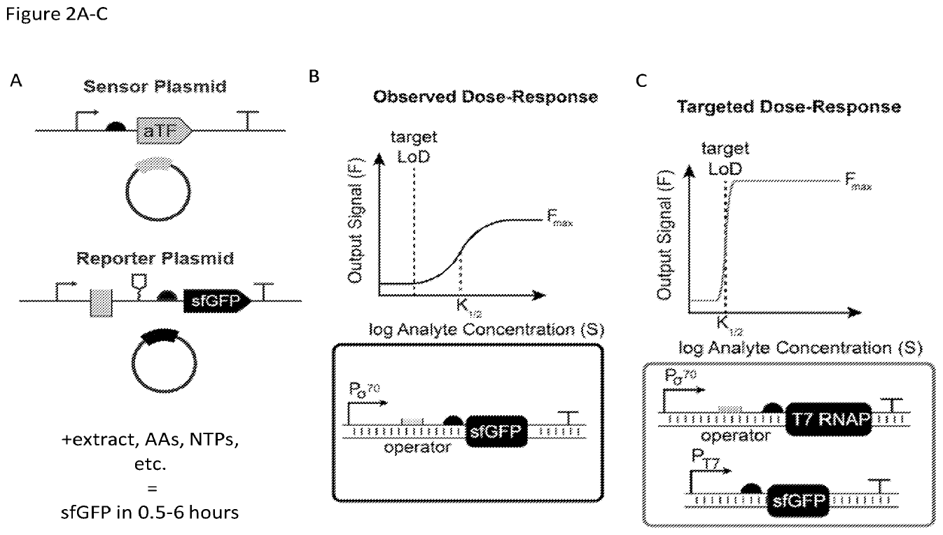

As used herein, the term “sensor plasmid” refers to a plasmid comprising a promoter which drives the expression of an allosteric transcription factor. See e.g., FIG. 2A. In some embodiments, the sensor plasmid is provided in an extract (e.g., as a component of a CFPS reaction), and the promoter is driven by the host's transcription and translation components). In some embodiments, the sensor plasmid is provided to a host strain, and the ATF protein is purified and added to the biosensor. Exemplary plasmids include but are not limited to pT7-CueR, pT7-MerR, pT7-ArsR, pT7-NarX, pT7-NarL, pT7-CadR.

The biosensor disclosed herein also comprise a reporter plasmid, or a linear reporter DNA construct. The reporter molecule can be any detectable protein. In some embodiments, the reporter protein can be visualized without the use of additional equipment or reagents. While GFP and sfGFP are exemplified herein, the biosensors are not intended to be so limited, and any number of detectable protein markers can be employed and include, but are not limited to green fluorescent protein, red fluorescent protein, blue fluorescent protein or any derivatives thereof. In some embodiments, the reporter comprises a den enzyme that produces a visible signal, such as catechol 2,3-dioxygenase (C23DO) beta-galactosidase (LacZ), or glucuronidase (GusA).

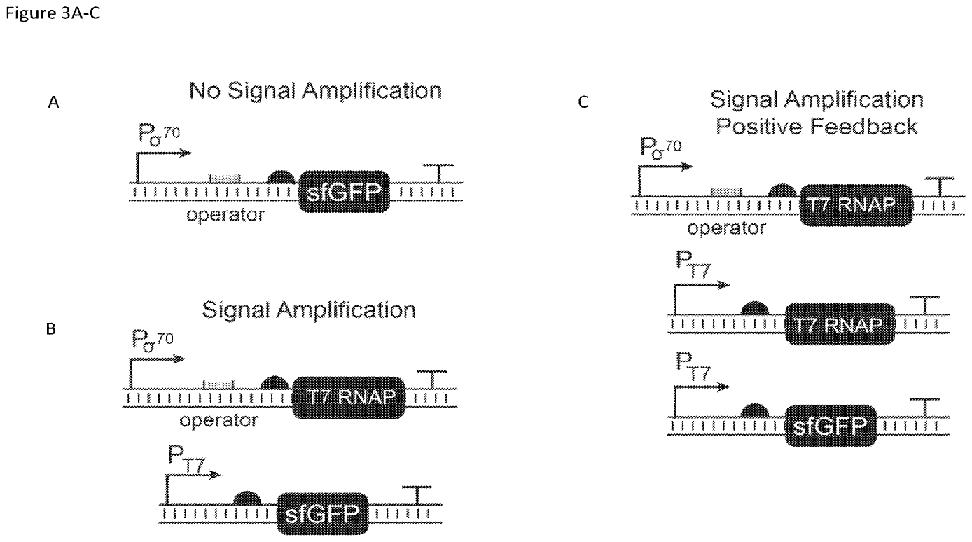

The reporter plasmid or linear construct also comprises a promoter to drive the expression of the reporter molecule. The promoter may be reactive to the ATF and its polymerase (e.g., as shown in FIG. 3A), or it may be reactive to an unrelated polymerase (e.g., as shown in FIGS. 3B and 3C).

The biosensors disclosed herein may also include one or more signal amplification constructs in the form of plasmid or linear DNA constructs (see e.g., FIGS. 3B and C). In the scheme outlined in FIG. 3B, the biosensor comprises (1) a biosensor molecule (e.g., an ATF protein), (2) a signal amplification plasmid or linear DNA construct (“amplifier” or “transducer”), and (3) a reporter plasmid or linear DNA construct. The ATF can be incorporated in the biosensor as a plasmid, a protein, or both e.g., an enriched extract. Likewise, the amplification and reporter constructs may also be added individually, or as part of an extract. In this embodiment, the amplifier construct comprises a promoter linked to an orthogonal polymerase. The ATF and its polymerase bind to the promoter on the amplifier and drive the expression of the orthogonal polymerase. The reporter construct comprises the matching orthogonal promoter linked to the reporter molecule. Exemplary plasmids include but are not limited to: pMer-AKSIRV, pArs-AKSIRV, pNar-AKSIRV, pFluor-AKSIRV, pCue-AKSIRV, pPbr-AKSIRV, pAKSIRV-RV.

In the scheme outlined in FIG. 3C, the biosensor comprises (1) a biosensor molecule (e.g., an ATF protein) (2) a “source” plasmid or linear DNA construct; (3) a signal amplification plasmid or linear DNA construct (“amplifier” or “transducer”), and (4) a reporter plasmid or linear DNA construct. Again, the ATF can be incorporated in the biosensor as a plasmid, a protein, or both e.g., an enriched extract. Likewise, the source, amplification, and reporter constructs may also be added individually, or as part of an extract. In this embodiment, the source construct comprises a promoter linked to a first orthogonal polymerase. The ATF and its polymerase bind to the promoter on the source construct and drive the expression of the first orthogonal polymerase. The amplifier construct comprises the matching first orthogonal promoter linked a second orthogonal polymerase. The first orthogonal polymerase binds to its promoter on the amplifier construct and drives expression of the second orthogonal polymerase. The reporter construct comprises the matching second orthogonal promoter linked to the reporter molecule. The second orthogonal polymerase binds to its promoter on the reporter construct and drives expression of the reporter molecule. In some embodiments, the first and second orthogonal polymerases are the same. Thus, in some embodiments, the presence of the target molecule increases the rate of transcription and translation of the additional RNA polymerase.

By way of example, allosteric transcription factors that are activated or deactivated by an interaction with another molecule include those shown below in Table 1. Their promoter sequences are also provided.

TABLE 1
Exemplary Allosteric Transcription Factors

| | Allo- | NCBI | |
|---|---|---|---|
| | steric | Ref- | |
| Acti- | Trans- | erence | |
| vator/ | cription | Se- | |
| Ligand | Factor | quence | Promoter Sequence |
| arsenic | ArsR EP3 | 562 | ACACATTCGTTAAGTCA |
| | mutant | | TATATGTTTTTGACTTA |
| | | | TCCGCTTCGAAGAGATA |
| | | | TAATACCTGCAA |
| | | | (SEQ ID NO: 7) |
| mercury | MerR | 83333 | ATCGCTTGACTCCGTAC |
| | | | ATGAGTACGGAAGTAAG |
| | | | GTTACGCTAT |
| | | | (SEQ ID NO: 8) |
| nitrate | NarX and | 83333 | (pydfJ115 hybrid |
| | Nar | | promoter from |
| | L | | Ekness, et. al. |
| | (pydfJ115 | | 2019 Nat. Chem. |
| | hybrid) | | Biol.): |
| | | | ACTGCATATTTGAAAAT |
| | | | TGCCCAAACGTACATGC |
| | | | CCGAATGTACGTTTTTT |
| | | | TCATTTCATTGTCAACT |
| | | | ACAATGAGAAAGAATGT |
| | | | GATCAAGCAATGTGTTG |
| | | | AAAGGAGATTATC |
| | | | (SEQ ID NO: 9) |
| copper | CueR | 83333 | TTCTTGACCTTCCCCTT |
| | | | GCtGGAAGGTTTATCCT |
| | | | CGGTT |
| | | | (SEQ ID NO: 10) |
| lead | PbrR | 470 | ATGTCTTGACTCTATAG |
| | | | TAACTAGAGGGTGTTAA |
| | | | ATCGGCA |
| | | | (SEQ ID NO: 11) |
| fluoride | crcB | 157 | TTGACAGCTAGCTCAGT |
| | ribo- | | CCTAGGTATAATACTAG |
| | switch | | TTTATAGGCGATGGAGT |
| | | | TCGCCATAAACGCTGCT |
| | | | TAGCTAATGACTCCTAC |
| | | | CAGTATCACTACTGGTA |
| | | | GGAGTCTATTTTTTT |
| | | | (SEQ ID NO: 12) |
| cadmium | CadR | 384676 | ATAACTTGACTCTGTAG |
| | | | TTGCTACAGGGTGTGCA |
| | | | ATCGGTT |
| | | | (SEQ ID NO: 13) |
| chromate | ChrB | 94626 | GTAGATCTTATCTCATT |
| | | | ATTGTAGTAATATCTAC |
| | | | (SEQ ID NO: 14) |

As described above, the promoter sequence responsive to the activated ATF drives the expression of a reporter molecule or an orthogonal polymerase. In some embodiments, the promoter sequence responsive to the activated ATF comprises an E. coli promoter sequence. By way of example, variants of the E. coli J23119 promoter sequence is used. Plasmids were assembled using isothermal (Gibson) assembly and confirmed by Sanger sequencing. The sequences for the ArsR (#78635) and MerR (#123148) genes were obtained from Addgene from Dr. Baojun Wang's lab. The sequences for the NarX and NarL plasmids were a generous gift from Dr. Jeffrey Tabor's lab. Other constitutive promoters engineered to have aTF-binding sites (e.g., operators grafted into or after the entire Anderson promoter collection in the BioBricks catalog) would be appropriate. The promoter can essentially be any promoter, so long as it is responsive to the selected ATF and the biosensor includes an appropriately matched polymerase.

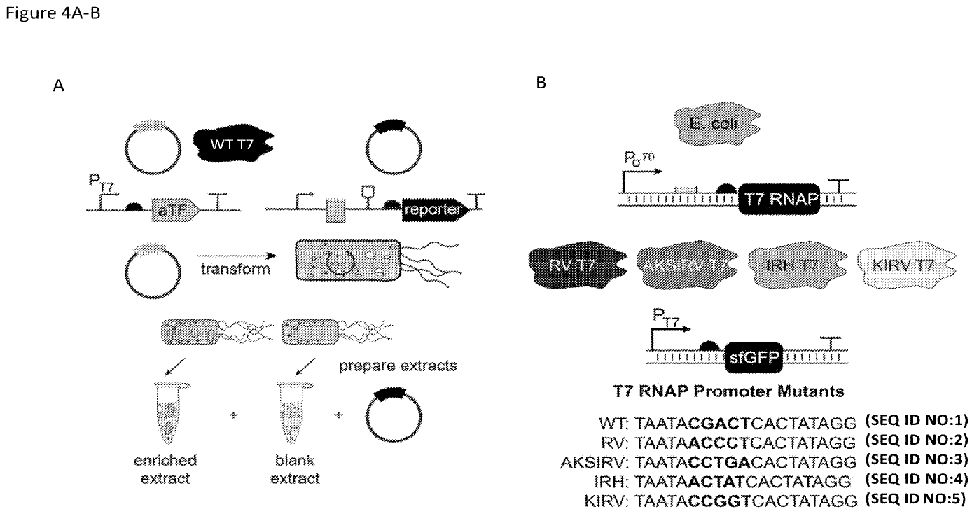

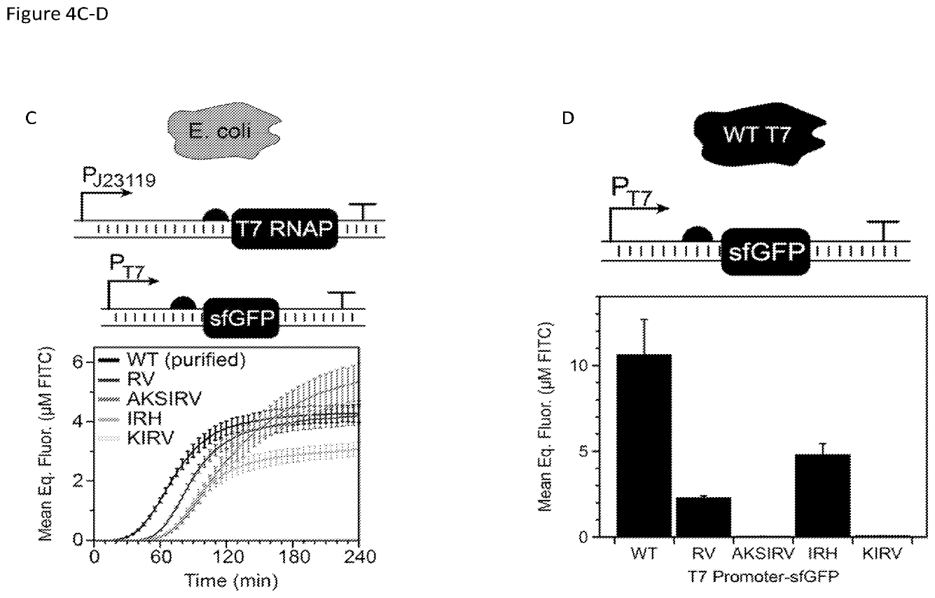

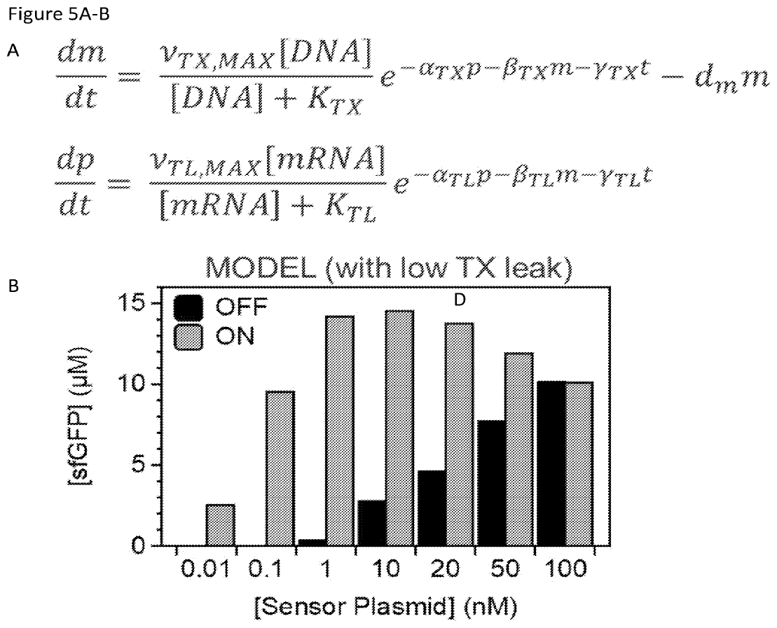

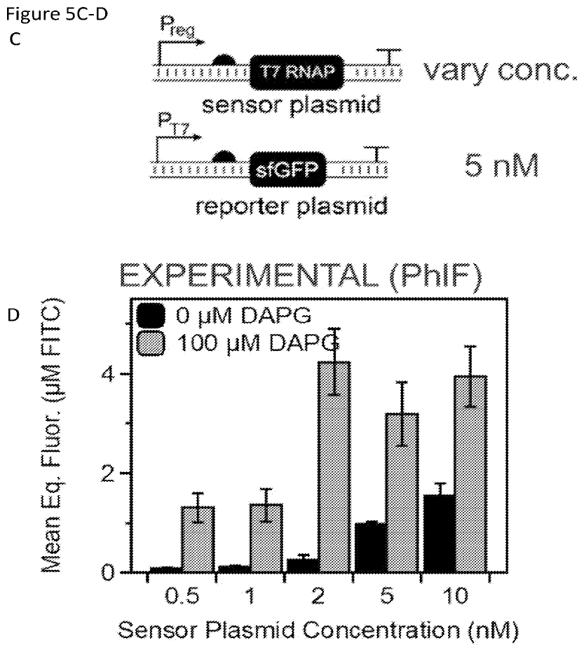

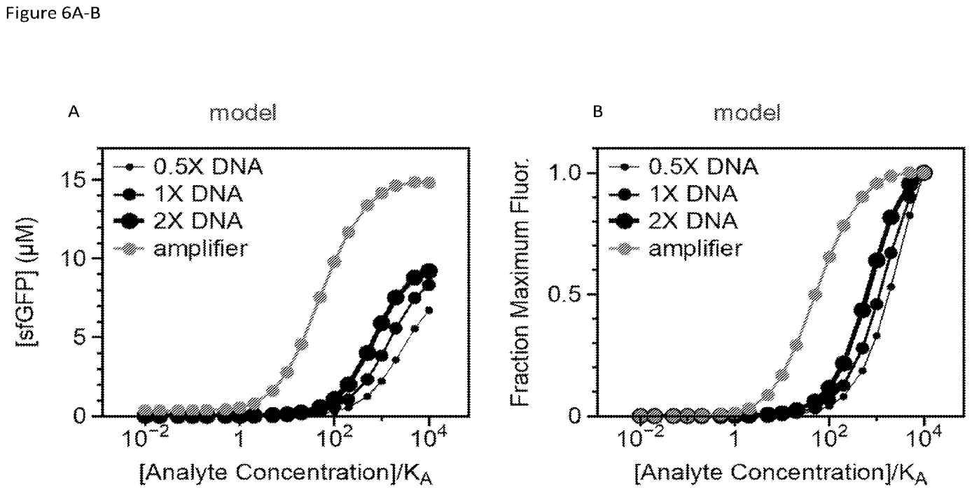

Orthogonal polymerases and matched promoters can be introduced in the biosensors to generate a cascade of polymerase transcription and translation (see e.g., FIGS. 3B and C). Such a cascade can enhance the time to signal generation (e.g. decrease detection time), and enhance signal generation (e.g., improve limits of detection and increase signal strength). In some embodiments, the additional RNA polymerase comprises a bacteriophage polymerase, e.g., from a bacteriophage in the podovirus family, such as T7 RNA polymerase, SP6 RNA polymerase and T3 RNA polymerase. In some embodiments, the additional polymerase comprises an engineered or evolved variant of the natural RNA polymerase. By way of example, several orthogonal polymerase mutants and their matched promoters, based on the bacteriophage T7 polymerase and promoter, are exemplified herein (see e.g., FIG. 4B), and are shown below in Table 2. Promoter variants were assembled by inverse PCR and blunt end ligation or Gibson assembly and confirmed by Sanger sequencing. The polymerases were assembled by Gibson assembly and were obtained as a generous gift from Dr. Ellington's lab on Addgene (#63627, 63628, 63629, and 63668).

TABLE 2
T7 promoter mutants and polymerase

| T7 | | Polymerase |
|---|---|---|
| Promo- | | [NCBI Reference |
| ter | | or other |
| | | reference |
| Mutant | Sequence | to identify] |
| WT | TAATACGACTCACTATAGG | 10760 |
| | (SEQ ID NO: 1) | |
| RV | TAATAACCCTCACTATAGG | RV polymerase |
| | (SEQ ID NO: 2) | from Meyer & |
| | | Ellington, |
| | | ACS Synth. |
| | | Biol. 2014. |
| AKSIRH | TAATACCTGACACTATAGG | AKSIRV |
| | (SEQ ID NO: 3) | polymerase |
| | | from Meyer & |
| | | Ellington, |
| | | ACS Synth. |
| | | Biol. 2014. |
| IRH | TAATAACTATCACTATAGG | IRH polymerase |
| | (SEQ ID NO: 4) | from Meyer & |
| | | Ellington, |
| | | ACS Synth. |
| | | Biol. 2014. |
| KIRV | TAATACCGGTCACTATAGG | KIRV polymerase |
| | (SEQ ID NO: 5) | from Meyer & |
| | | Ellington, |
| | | ACS Synth. |
| | | Biol. 2014. |

By way of example but not by way of limitation, expression vectors that can be used in the methods and systems disclosed herein are provided in Table 3 below.

TABLE 3
Exemplary Expression Constructs

| Construct | Construct NAME | SEQ ID NO: |
|---|---|---|
| aTFs | pT7-ArsR-EP3 | SEQ ID NO: 15 |
| | pT7-CadR | SEQ ID NO: 16 |
| | pT7-CueR | SEQ ID NO: 17 |
| | pT7-MerR | SEQ ID NO: 18 |
| | pT7-NarL-hybrid | SEQ ID NO: 19 |
| | pT7-NarX | SEQ ID NO: 20 |
| | pT7-PbrR | SEQ ID NO: 21 |
| Cascade Reporters | pAKSIRV-sfGFP | SEQ ID NO: 22 |
| | pAKSIRV-XylE | SEQ ID NO: 23 |
| | pIRH-sfGFP | SEQ ID NO: 24 |
| | pKIRV-sfGFP | SEQ ID NO: 25 |
| | pRV-sfGFP | SEQ ID NO: 26 |
| | pT7-sfGFP | SEQ ID NO: 27 |
| Cascade Sensors | crcB-AKSIRV | SEQ ID NO: 28 |
| | J23119- | SEQ ID NO: 29 |
| | AKSIRV_low_copy | |
| | J23119-IRH | SEQ ID NO: 30 |
| | J23119-KIRV | SEQ ID NO: 31 |
| | J23119-RV | SEQ ID NO: 32 |
| | pArs-AKSIRV | SEQ ID NO: 33 |
| | pCad-AKSIRV | SEQ ID NO: 34 |
| | pMer-AKSIRV | SEQ ID NO: 35 |
| | pNar-AKSIRV | SEQ ID NO: 36 |
| Multilayer Cascades | pAKSIRV-AKSIRV | SEQ ID NO: 37 |
| | pAKSIRV-AKSIRV- | SEQ ID NO: 38 |
| | LET | |
| | pAKSIRV-RV | SEQ ID NO: 39 |
| Non-Cascaded | crcB-fGFP | SEQ ID NO: 40 |
| Sensors | pArs-sfGFP | SEQ ID NO: 41 |
| | pCad-sfGFP | SEQ ID NO: 42 |
| | pCue-sfGFP | SEQ ID NO: 43 |
| | pMer-sfGFP | SEQ ID NO: 44 |
| | pNar-sfGFP | SEQ ID NO: 45 |
| | pPbr-sfGFP | SEQ ID NO: 46 |

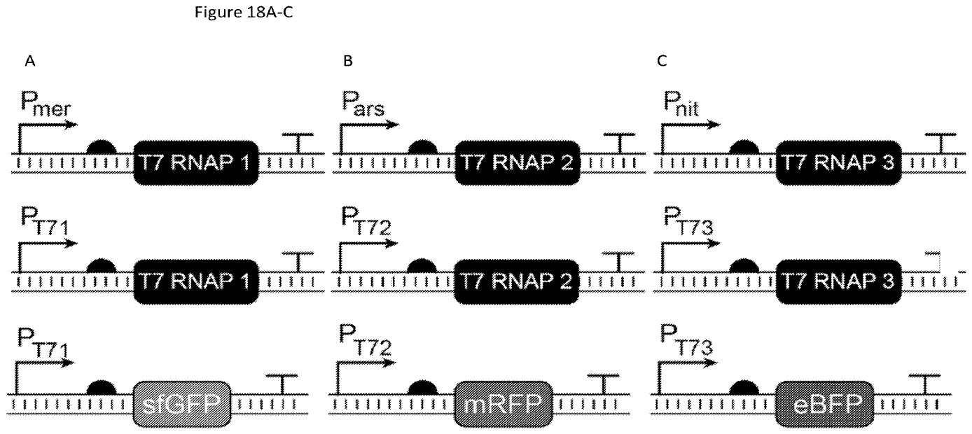

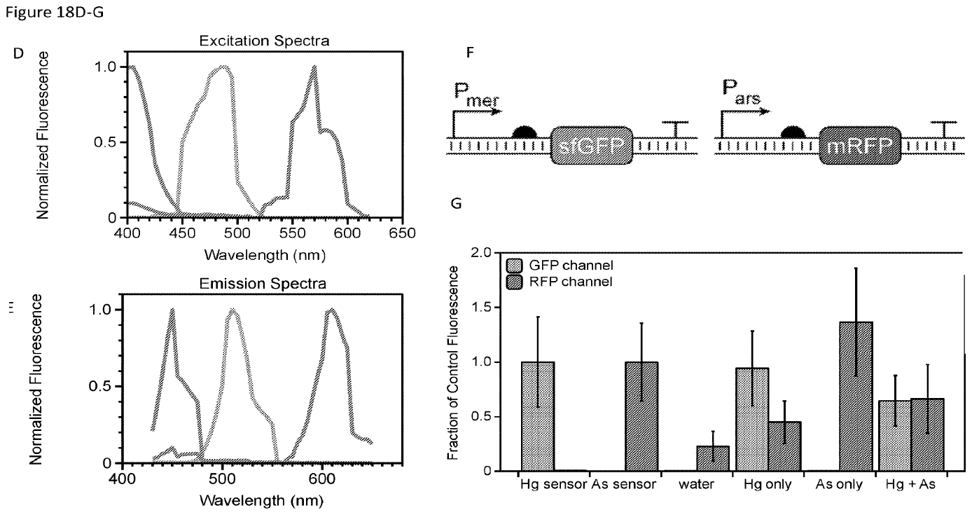

The biosensors disclosed herein may be multiplexed; that is, more than one target can be detected in a single reaction vessel. By way of example, as shown in FIG. 18, by using different combinations of ATFs, amplification, and detection polymerases, multiple targets can be detected in a single reaction. Thus, in some embodiments, the compositions, kits, and systems disclosed herein include (a) a lysate from an engineered bacterial strain, the lysate comprising cellular transcriptional and translational machinery, and optionally, other cellular proteins, cofactors, and energy sources; (b) two or more DNA transcription templates encoding an additional RNA polymerase not present in the lysate and configured to be conditionally expressed (e.g., in the presence of the a target molecule); and (c) two or more DNA transcriptional templates encoding the expression of a reporter molecule under control of transcription by an additional RNA polymerase not present in the lysate. Thus in some embodiments, multiple target molecules can be detected in a single tube by using orthogonal RNA polymerases.

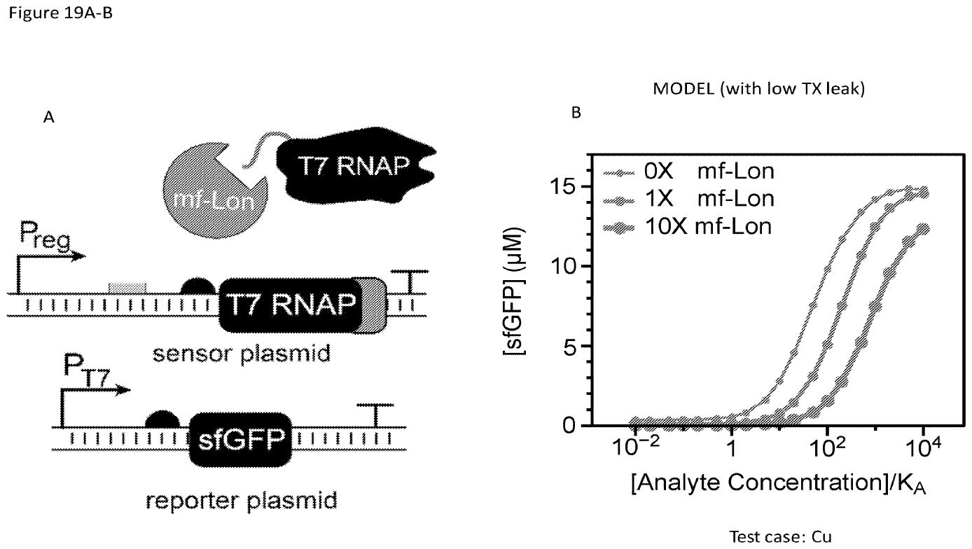

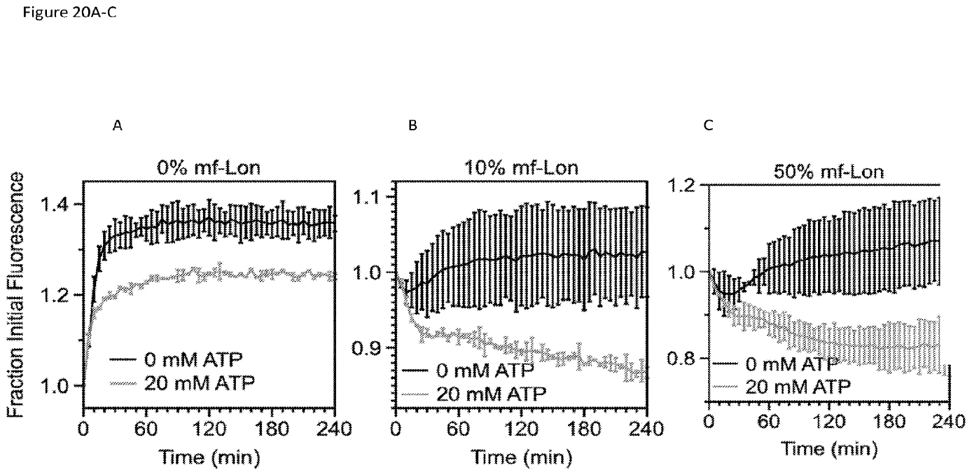

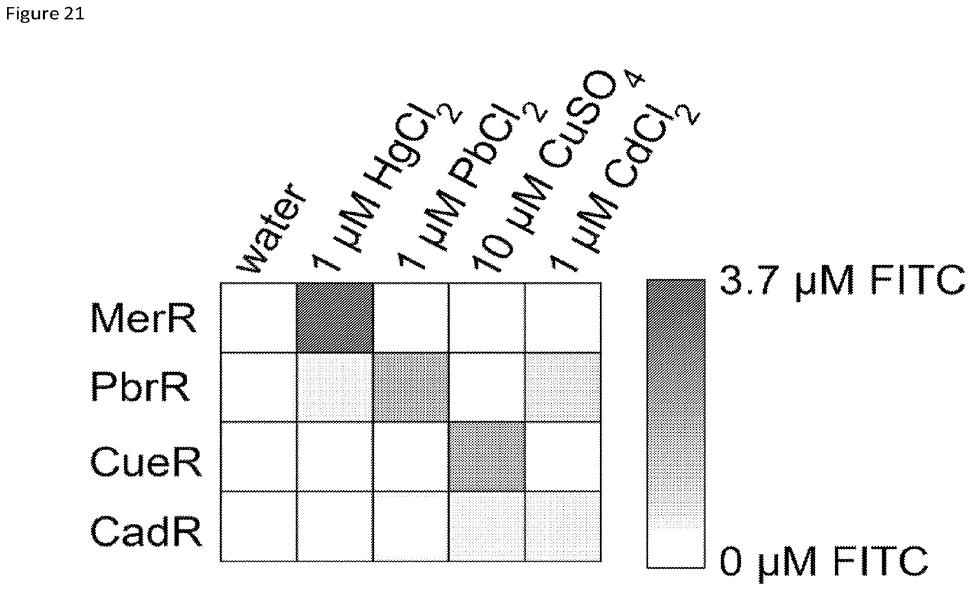

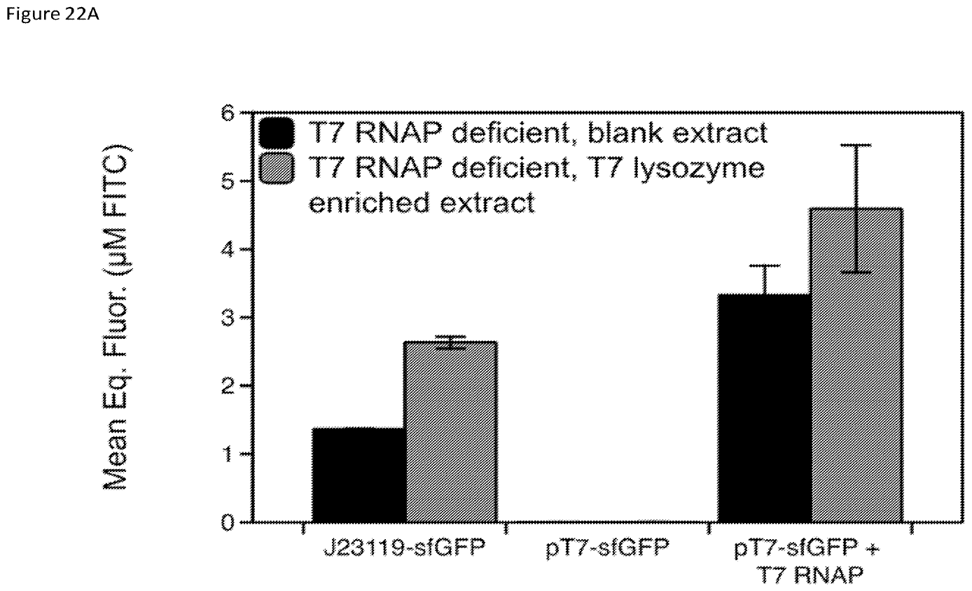

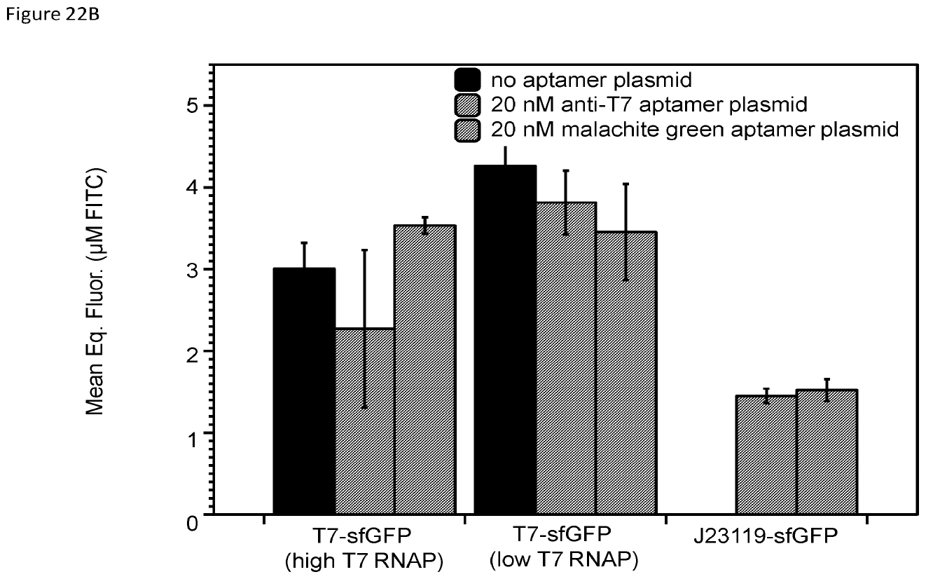

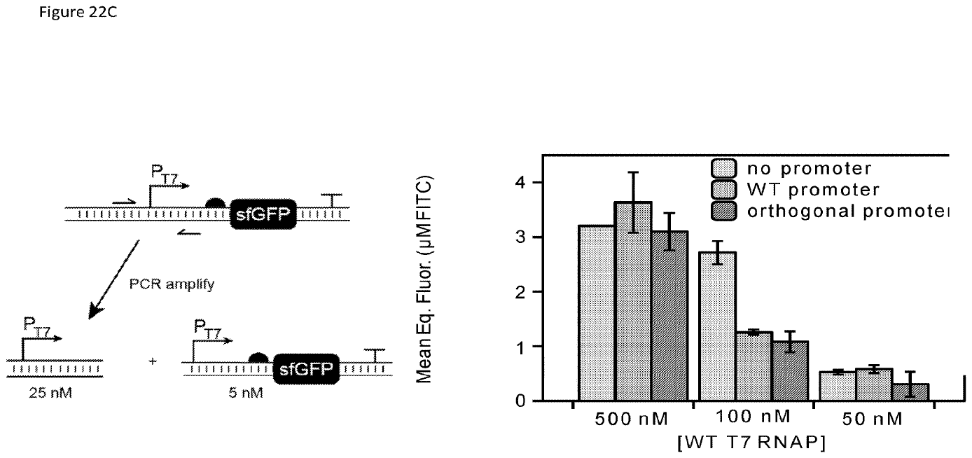

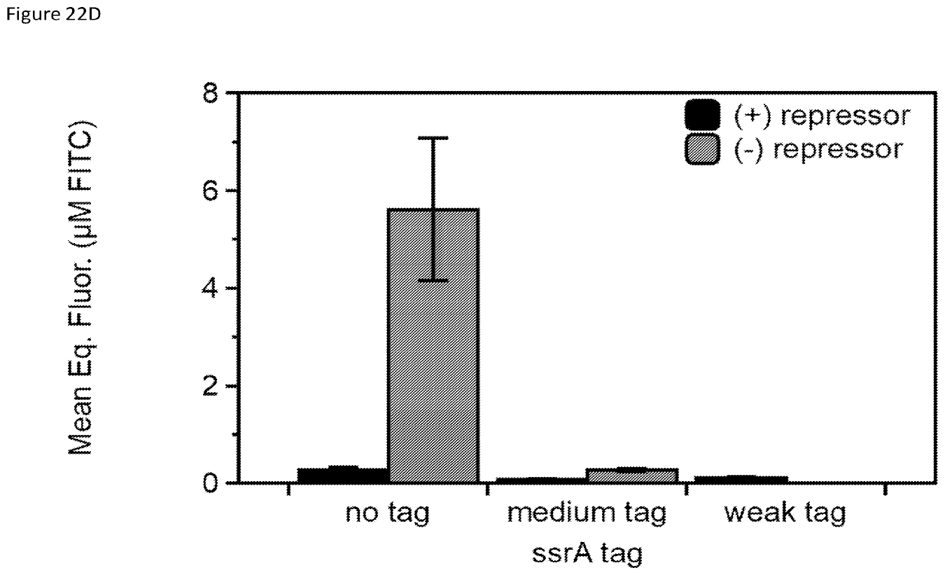

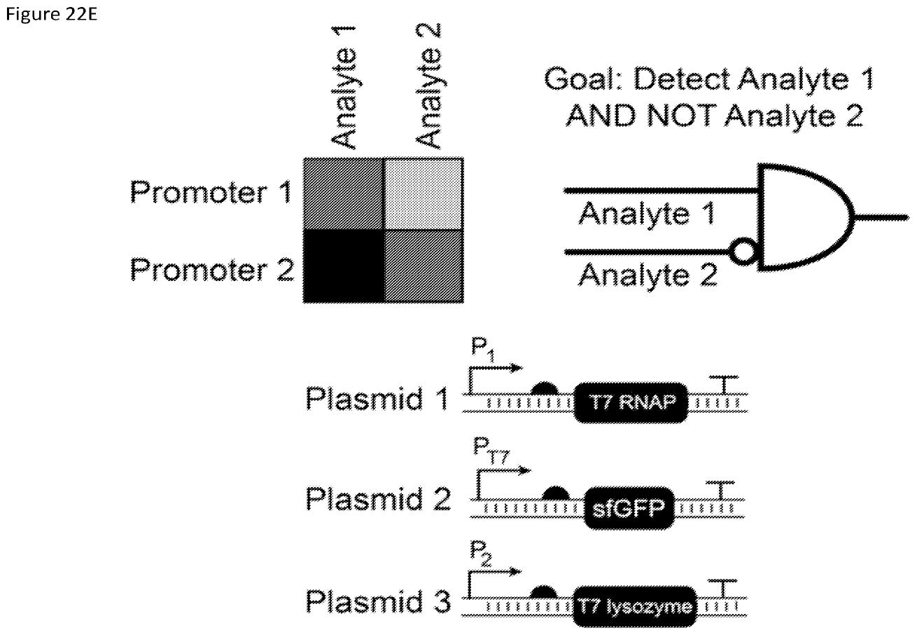

In some embodiments, the biosensors are optimized, e.g., to provide detectable signals in a shorter time, and/or cleaner signal (e.g., with less background). Cascaded systems, as described above, provide one means of optimization. Another means of optimization includes regulating the T7 polymerase activity and/or expression. As shown in FIGS. 19-22, several options can be employed, including T7 lysozyme, aptamers, promoter mimics, and targeted protein degradation (e.g., using AAA+ proteases such as ClpX, Lon, ClpAP, HslUV, and FtsH). With regard to targeted protein degradation, the protein of interest is modified to include a specific tag (e.g., SsrA; Sul20C, etc.), recognized by its protease. In some embodiments, the amount of the protease can be tightly controlled, e.g., by adding the purified or partially purified protease to the extract/reaction mixture.

In some embodiments, optimization includes “additional” RNA polymerases that have been specifically evolved or engineered for specificity for only a single promoter to avoid crosstalk.

In some embodiments, optimization includes reporter protein comprising orthogonal fluorescence or absorbance spectra, or catalyze enzymatic reactions that produce different colors.

In some embodiments, the target molecule to be detected comprises one or more of phloroglucinol, mercury, arsenic or its oxides, nitrate, fluoride, cyanuric acid, lead, copper, zinc, chromium or its oxides or atrazine. In some embodiments, the target molecule to be detected comprises RNA or DNA.

In some embodiments, the nucleic acids provided as components of the biosensor are amplified using an isothermal strategy prior to sensor activation. By way of example, nucleic acid sequence-based amplification (NASBA) and recombinant polymerase amplification (RPA) may be used.

Methods employing the biosensors are also contemplated herein. For example, methods of detecting a target molecule (e.g., a metabolite, a chemical compound, a nucleic acid) in a biological or environmental sample may include: (i) obtaining a biological or environmental sample which may or may not contain the target molecule and optionally concentrating and/or solubilizing the target molecule in the sample if necessary; (ii) adding the sample and/or the optionally concentrated and/or solubilized target molecule in the sample to a cell-free protein synthesis (CFPS) reaction, wherein if the target molecule is present in the sample then an output is generated (e.g., a visual, electronic, or optical output); wherein the output is generated via steps that include: (i) the target molecule inducing expression of an RNA polymerase from a first DNA transcription template, wherein the expressed RNA polymerase is not present in the CFPS reaction prior to its expression, optionally wherein the expression of the RNA polymerase is induced via a biosensor molecule in the presence of the target molecule; (ii) the expressed RNA polymerase expresses a reporter molecule from a second DNA transcription template (e.g., wherein the second DNA transcription template comprises a promoter for the expressed RNA polymerase) and the reporter molecule generates an output either directly or indirectly.

### Applications and Advantages

Applications of the disclosed technology may include but are not limited to: (i) improving the sensitivity of molecular diagnostics, such as field-deployable molecular diagnostics; (ii) improving the maximum detectable signal of molecular diagnostics; (iii) improving the response transfer curve of molecular diagnostics for more sensitive and sigmoidal switching behavior; and (iv) enabling one-pot multiplexing of several cell-free sensors.

Advantages of the disclosed technology may include but are not limited to: (i) the development of sensors having an improved limit of detection for arbitrary analytes (target molecules) which is enhanced compared to a no-signal amplification condition, as well as the reporter signal in the ON (i.e. target chemical present) state; (ii) the development of sensors having a response which is more “switchlike”, or sigmoidal, enabling better semi-quantitative determination of concerning concentrations of relevant analytes; and (iii) the development of sensors which are extremely modular and adapted to various reporter outputs, which also enables one-pot multiplexing of various detection schemes with different fluorescent proteins or enzymes. Point-of-care, field-deployable diagnostics could allow consumers to rapidly and inexpensively determine water quality, be used for personalized health monitoring, be used for point-of-use health monitoring.

### EXAMPLES

The examples provided herein are not intended to be limiting, and are provided to demonstrate aspects of the present technology.

### Example 1—Cell-Free Sensors as Point of Care Diagnostics

In general, a cascaded and noncascaded sensor requires three plasmids that are designed and assembled using standard molecular biology strategies (isothermal assembly, restriction cloning, blunt-end ligation, solid-phase oligonucleotide synthesis, etc.). One plasmid encodes the target allosteric transcription factor (e.g., CueR) under the control of the wild-type T7 RNAP promoter. The other two encode the responsive promoter sequence (e.g., pCue) upstream of a reporter, either the sfGFP coding sequence (the noncascaded sensor) or T7 RNAP (the cascaded sensor). The natural promoter sequences are typically used, although for promoters derived from non-E. coli hosts, mutations to the consensus −10 and −35 sites for sigma-70 promoters (TTGACA, TATAAT) can be helpful to generate stronger promoters that are still functionally regulated. Multiple operator sites can also be placed in tandem in a promoter to improve the ability of the aTF to regulate gene expression. The same cascaded reporter plasmid (e.g., pAKSIRV-sfGFP) is used for all experiments described herein.

The optimized reporter plasmids are isolated and sequence-confirmed and the reporter plasmids are purified to high concentration by midiprep. The plasmid encoding the aTF is transformed into a protein expression strain of E. coli (e.g., BL21 (DE3) or its derivatives) and an extract is prepared using previously reported protocols (e.g., citation 18). Separately, a “blank” extract is prepared from the base protein production strain. Cell-free extracts for transcriptional sensing are typically prepared by lysis and post-lysis clarification including ribosomal runoff reaction and dialysis, followed by aliquoting and flash-freezing on liquid nitrogen.

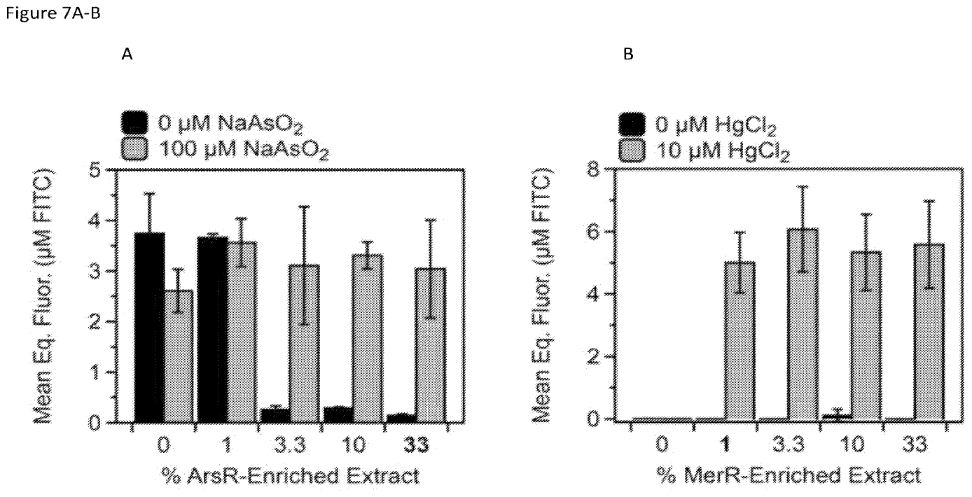

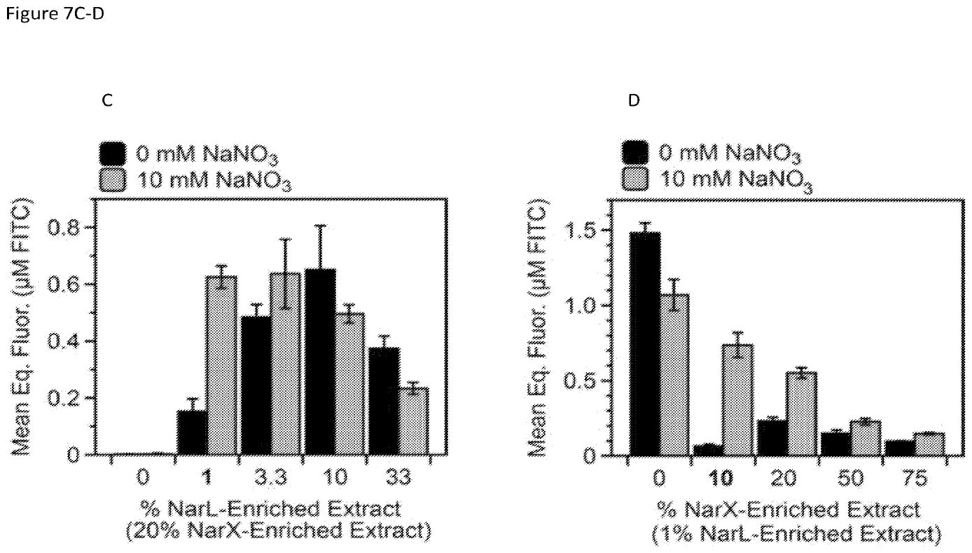

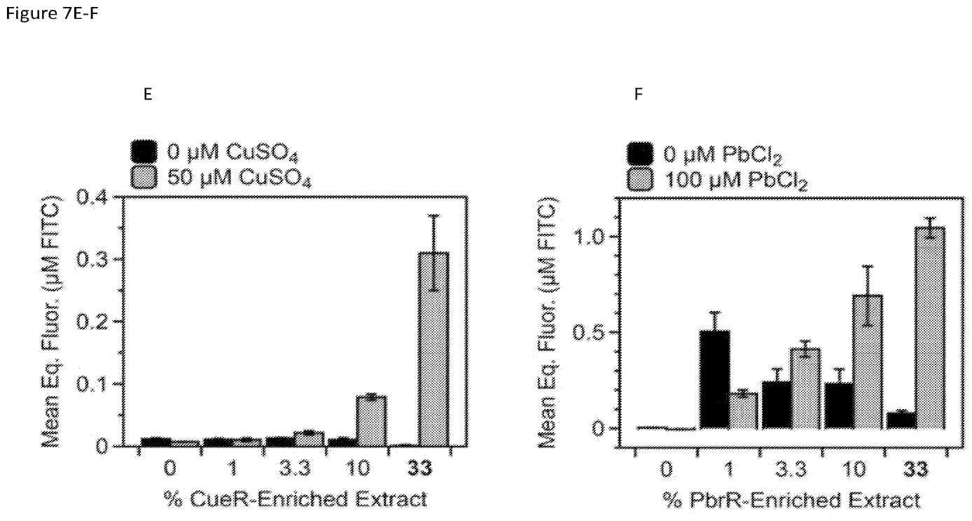

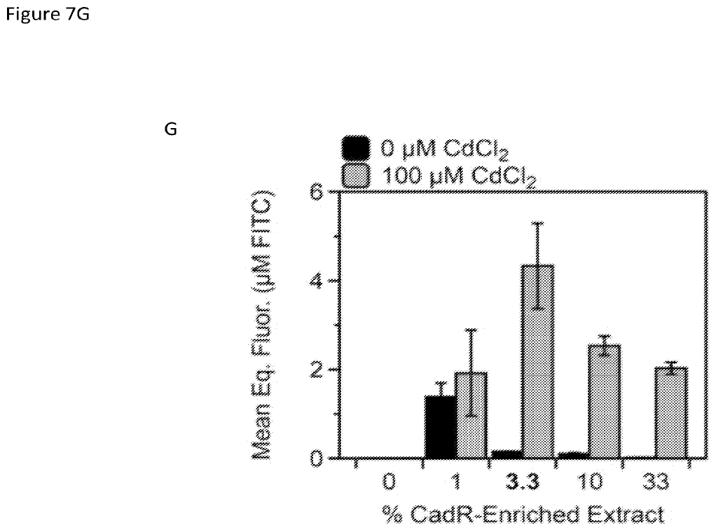

A series of tuning experiments are next used to optimize the sensor. First, the optimal concentration of the aTF (CueR) is found for the non-cascaded sensor (e.g., pCue-sfGFP) through a series of ratiometric titrations between the aTF-enriched extract and the blank extract in both the presence and absence of analyte (see e.g., the data in FIG. 7). This experiment is an isothermal cell-free gene expression reaction held at 30° C. in a plate reader for −4 hours using an established set of physiochemical conditions to maximize protein production in general. It may also be necessary to determine the concentration of analyte that saturates the cell-free sensor without inhibiting transcription and translation in vitro, which can be done with a simple titration of analyte against an unregulated reporter plasmid (e.g., pT7-sfGFP). For example, 100 μM CuSO₄ shuts down cell-free protein synthesis.

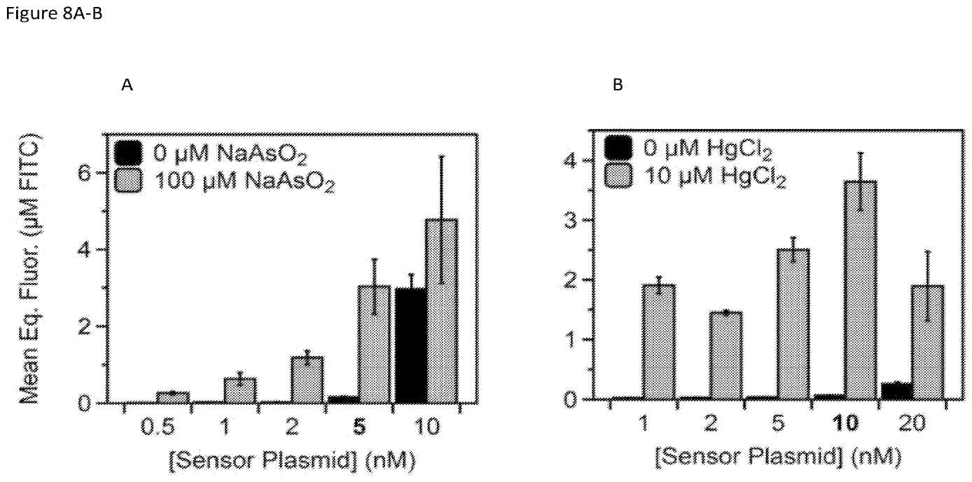

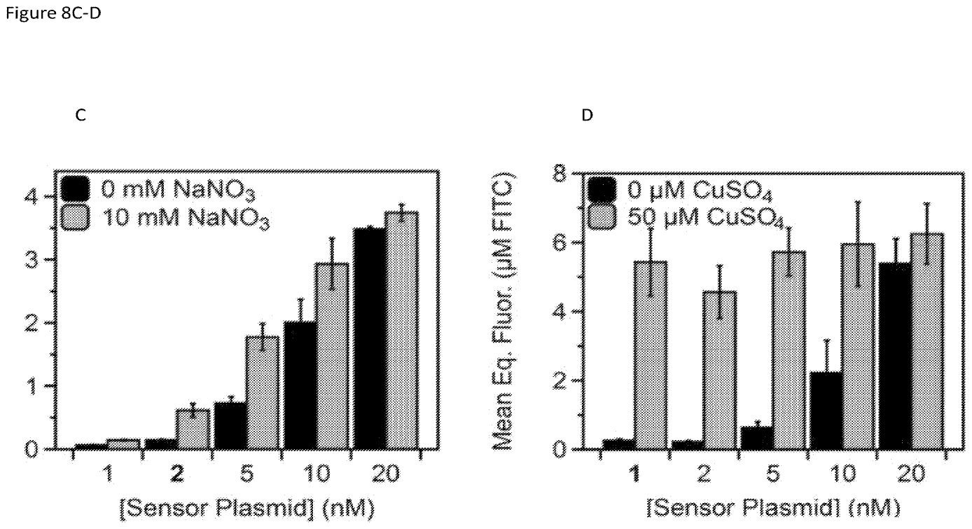

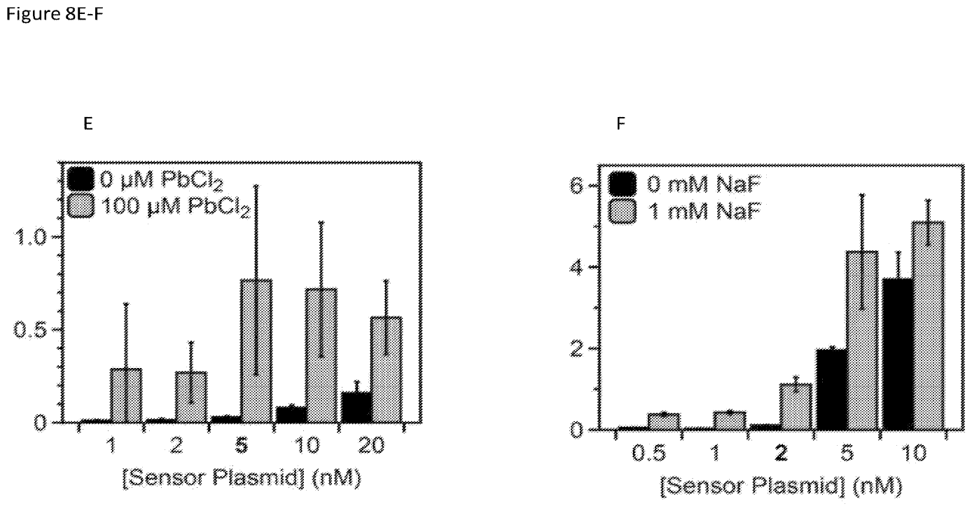

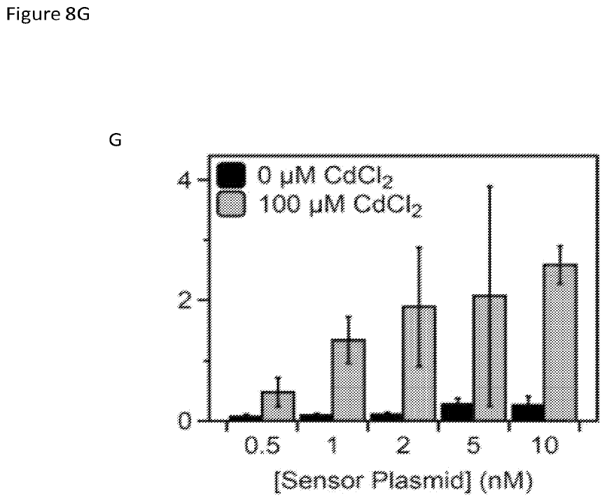

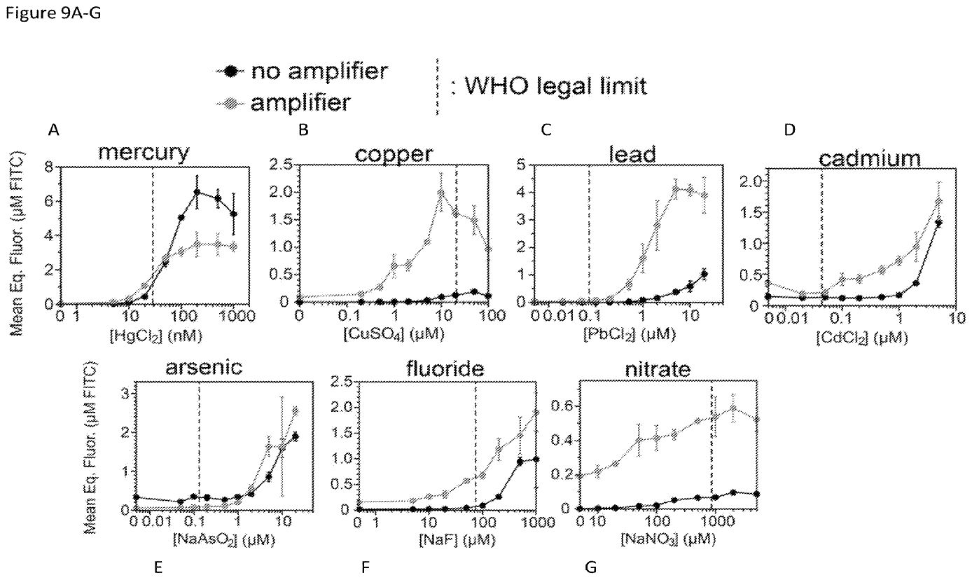

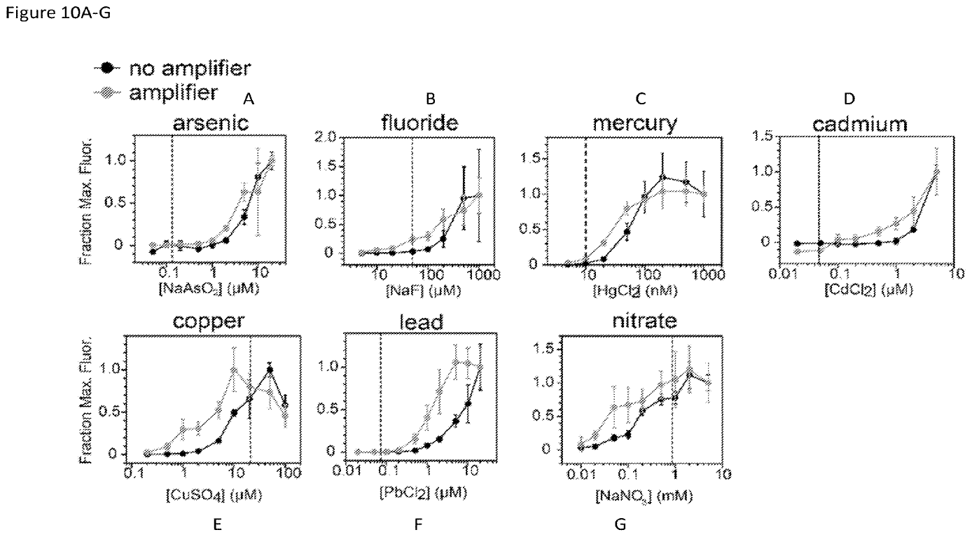

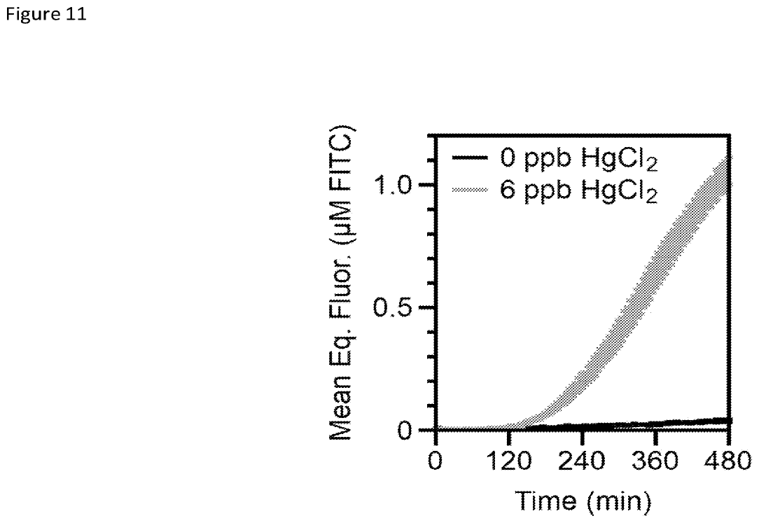

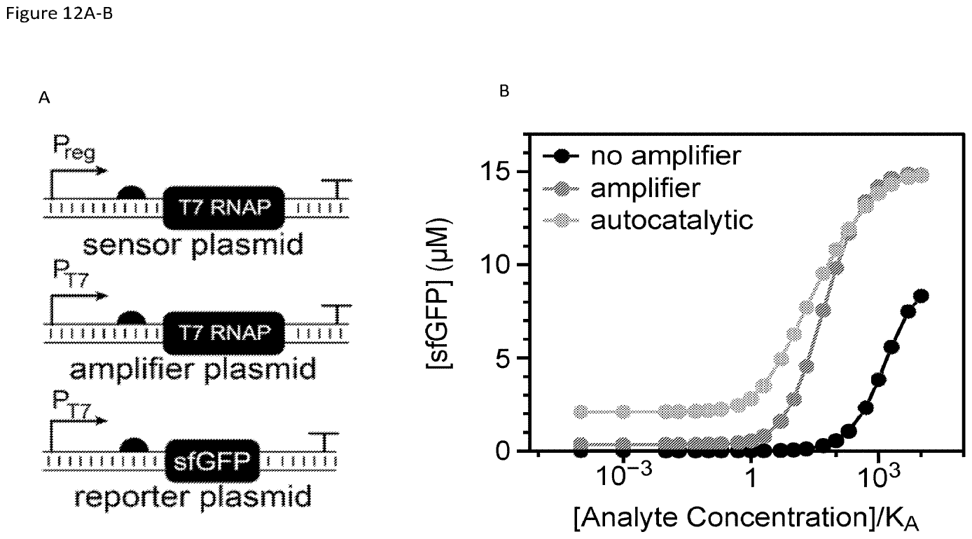

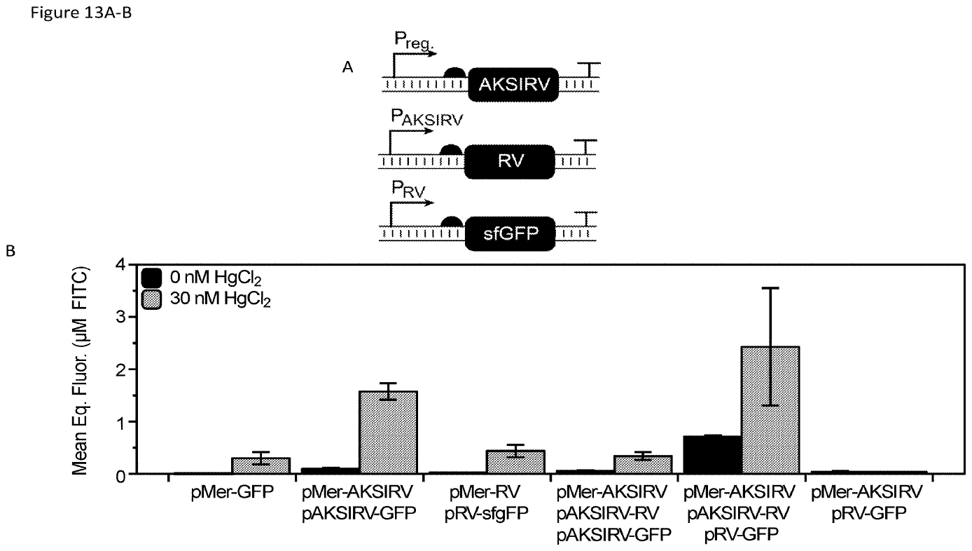

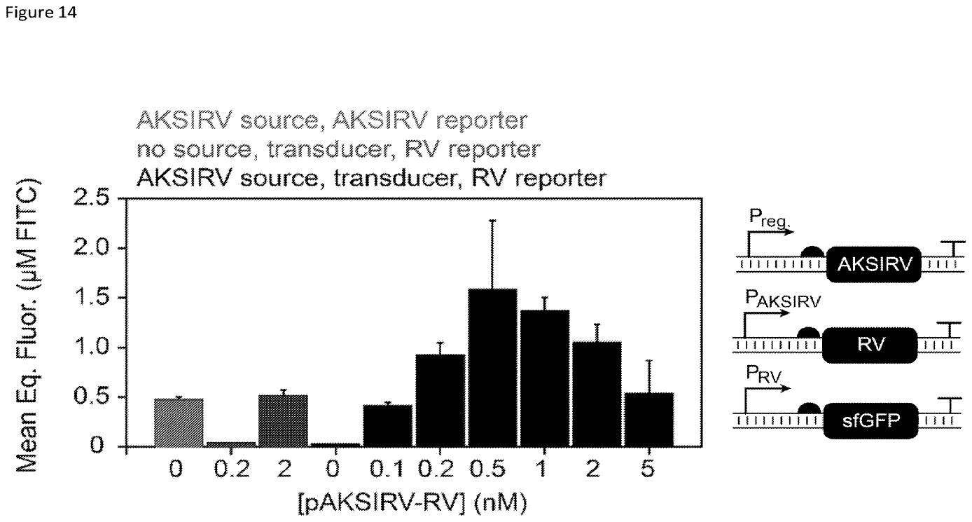

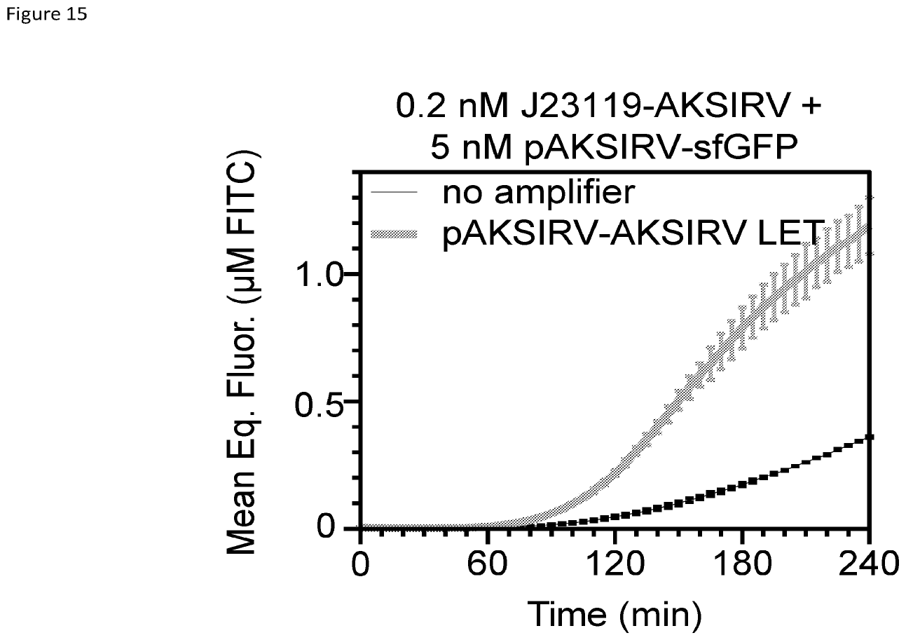

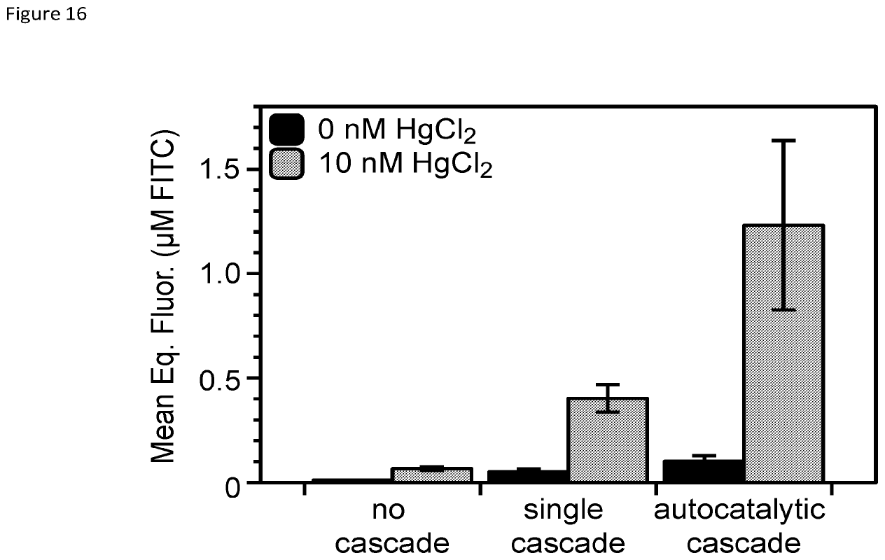

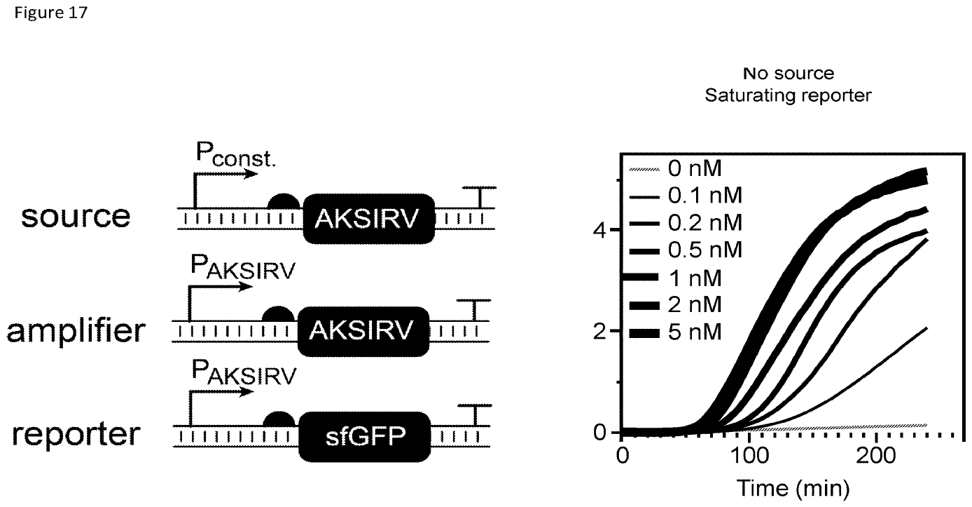

Next, the optimal ratio of aTF enriched- and blank extract is used to optimize the concentration of the sensor plasmid sequence for the cascade (e.g., pCue-AKSIRV) (see e.g., the data in FIG. 8). In each case, the goal is to optimize the fold activation of the sensor. Typically the concentration of the reporter plasmid is held constant and saturating for both the noncascaded sensor (20 nM) and the cascaded sensor (5 nM), although this can also be separately optimized to maximize fold activation. Then the dose responses are measured (see e.g., FIG. 9).

The innovations disclosed herein, include but are not limited to: (i) deploying a cell-free sensor that produces a bacteriophage RNA polymerase in the presence of a target chemical or nucleic acid; (ii) catalytic amplification of that bacteriophage polymerase with a positive feedback template, and co-expression of a reporter protein from the bacteriophage polymerase's cognate promoter, in one pot; and (iii) deploying multiple engineered polymerase variants in a single pot which allows for multiplexed detection of several analytes at once. The disclosed innovations allow for overall improved signal of the sensor and also makes the sensor be more switchlike, greatly increase the limit of detection of the disclosed sensors; and allow for practical sensing of several contaminants using a single reaction, which simplifies the device and decreases overall cost. The exemplary models and designs presented herein uniquely demonstrated the ability of the cascaded amplifiers to be applied to and monitor lead, mercury, nitrate, cadmium, copper, fluoride, chromate, and arsenic.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

### Non-Patent References

1. Pardee, K. et al. Paper-Based Synthetic Gene Networks. Cell 1-22 (2014). doi:10.1016/j.cell.2014.10.004
2. Pardee, K. et al. Rapid, Low-Cost Detection of Zika Virus Using Programmable Biomolecular Components. Cell 165, 1-25 (2016).
3. Salehi, A. S. M. et al. Biosensing estrogenic endocrine disruptors in human blood and urine: A RAPID cell-free protein synthesis approach. Toxicol. Appl. Pharmacol. 345, 19-25 (2018).
4. Peter L Voyvodic, Amir Pandi, Mathilde Koch, Jean-Loup Faulon, Jerome Bonnet. Plug-and-Play Metabolic Transducers Expand the Chemical Detection Space of Cell-Free Biosensors. doi: https://doi.org/10.1101/397315
5. Gootenberg, J. S. et al. Nucleic acid detection with CRISPR-Cas13a/C2c2. Science 356, 438-442 (2017).
6. Pandi, A., et al., Optimizing Cell-Free Biosensors to Monitor Enzymatic Production. ACS Synthetic Biology, 2019. 8(8): p. 1952-1957.
7. Verosloff, M., et al., PLANT-Dx: A Molecular Diagnostic for Point-of-Use Detection of Plant Pathogens. ACS Synthetic Biology, 2019. 8(4): p. 902-905.
8. Pellinen, T., T. Huovinen, and M. Karp, A cell-free biosensor for the detection of transcriptional inducers using firefly luciferase as a reporter. Analytical Biochemistry, 2004. 330(1): p. 52-57.
9. Alam, K. K., et al., Rapid, Low-Cost Detection of Water Contaminants Using Regulated In Vitro Transcription. bioRxiv, 2019: p. 619296.
10. Thavarajah, W., et al., Point-of-Use Detection of Environmental Fluoride via a Cell-Free Riboswitch-Based Biosensor. bioRxiv, 2019: p. 712844.
11. McNerney, M. P., et al., Point-of-care biomarker quantification enabled by sample-specific calibration Science Advances, 2019.
12. Liu, X., et al., Design of a transcriptional biosensor for the portable, on-demand detection of cyanuric acid. bioRxiv, 2019: p. 736355.
13. Grawe, A., et al., A paper-based, cell-free biosensor system for the detection of heavy metals and date rape drugs. PLOS ONE, 2019. 14(3): p. e0210940.
14. Takahashi, M. K., et al., A low-cost paper-based synthetic biology platform for analyzing gut microbiota and host biomarkers. Nature Communications, 2018. 9(1): p. 3347.
15. Garamella, J., et al., The All E. coli TX-TL Toolbox 2.0: A Platform for Cell-Free Synthetic Biology. ACS Synthetic Biology, 2016. 5(4): p. 344-355.
16. Meyer, A. J., J. W. Ellefson, and A. D. Ellington, Directed Evolution of a Panel of Orthogonal T7 RNA Polymerase Variants for in Vivo or in Vitro Synthetic Circuitry. ACS Synthetic Biology, 2015. 4(10): p. 1070-1076.
17. Pandi, A., et al., Metabolic perceptrons for neural computing in biological systems. Nature Communications, 2019. 10(1): p. 3880.
18. Silverman, A. D., et al., Design and optimization of a cell-free atrazine biosensor. bioRxiv, 2019: p. 779827.
19. Wan, X., et al., Cascaded amplifying circuits enable ultrasensitive cellular sensors for toxic metals. Nature Chemical Biology, 2019. 15(5): p. 540-548.
20. Temme, K., et al., Modular control of multiple pathways using engineered orthogonal T7 polymerases. Nucleic acids research, 2012. 40(17): p. 8773-8781.

### Patent References

U.S. Pat. Nos. 5,478,730; 5,556,769; 5,665,563; 6,168,931; 6,518,058; 6,783,957; 6,869,774; 6,994,986; 7,118,883; 7,189,528; 7,338,789; 7,387,884; 7,399,610; 8,357,529; 8,574,880; 8,703,471; 8,999,668; 9,410,170; and US952813; the contents of which are incorporated herein by reference in their entirety.
U.S. patenttent Publications: US20040209321; US20050170452; US20060211085; US20060234345; US20060252672; US20060257399; US20060286637; US20070026485; US20070154983; US20070178551; US20080138857; US20140295492; US20160060301; US20180016612; US20180016614; US20160312312; and US20160362708; the contents of which are incorporated herein by reference in their entirety.
Published International Applications: WO2003056914A1; WO2004013151A2; WO2004035605A2; WO2006102652A2; WO2006119987A2; WO2007120932A2; WO2014144583; and WO2017117539; the contents of which are incorporated herein by reference in their entirety.

### Example 2. Improved Cell-Free Lead Biosensor

Disclosed are cell-free systems for molecular sensing of metals based on responsive transcription factors, methods for engineering cell-free transcription factor protein sequences using iterative saturation mutagenesis screening, and novel protein sequences for a lead sensing protein based on PbrR from Cupriavidus metallidurans (GenBank: ABF12805.1). The disclosed systems and methods may be utilized for sensing.

### Exemplary Applications and Advantages

On-demand biosensing of molecules in enriched bacterial extracts using fluorescent and colorimetric reporters; high-throughput screening of transcription factor sequence variants for improved dynamic range, limit of detection, and promiscuity; method is able to look at all possible mutational possibilities at each residue which is a more thorough interrogation of mutational search space and more likely to yield a beneficial result; rapid detection of target molecule over background using fluorescent or colorimetric measurement; compared to whole-cell biosensors, little-to-no concern for biocontainment or regulations on transgenic organisms, issues with ligand permeability or degradation inside cells, or cytotoxicity.

### Exemplary Commercial Problem to be Solved

Lack of easy-to-use on-site tests for contaminants; chemical test can be expensive and/or produce additional environmental contaminants.

The state of the art for official testing is analytical chemistry, as it uses machines such as GC-MS to detect even trace amounts of most compounds. However, one has to ship preserved samples to a centralized facility that houses this equipment, which is inaccessible in resource limited settings. One proposed solution are whole-cell biosensors, which repurpose naturally evolved proteins called allosteric transcription factors (also abbreviated aTF) to sense chemicals and then produce some genetic signal output in return. However, the use of live hosts introduce biocontainment issues, toxicity concerns, and equipment required to culture and grow the cells.

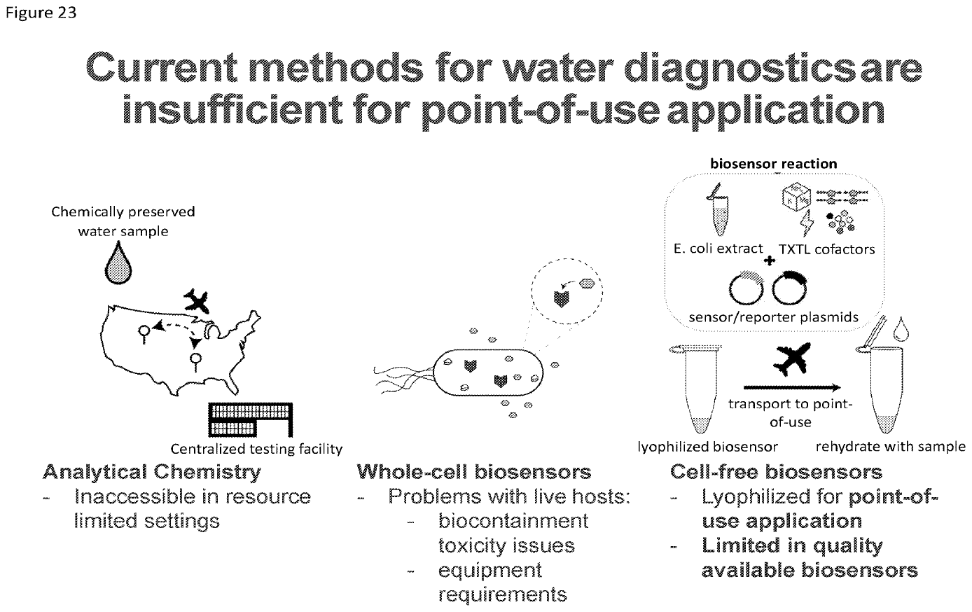

The platform disclosed herein includes cell-free biosensors. These biosensors remove the bacteria chassis by lysing the cell to generate cellular extract or lysate. Extracts aren't alive, but retain the cell's native transcription and translation abilities for in vitro protein synthesis. By way of example, a cell free reaction can be generated by combining E. coli extract, TXTL cofactors, and the aTF sensor and reporter encoded on plasmids. Cell-free sensors remove the live host downsides from whole-cell biosensors, while giving the added benefits of an open reaction environment, easily scaled for high-throughput reactions, and which can be lyophilized for transport and rehydrated with a water sample at the point of use with minimal loss in biosensor function. See FIG. 23.

There are many factors that may be considered for developing a “good” sensor.

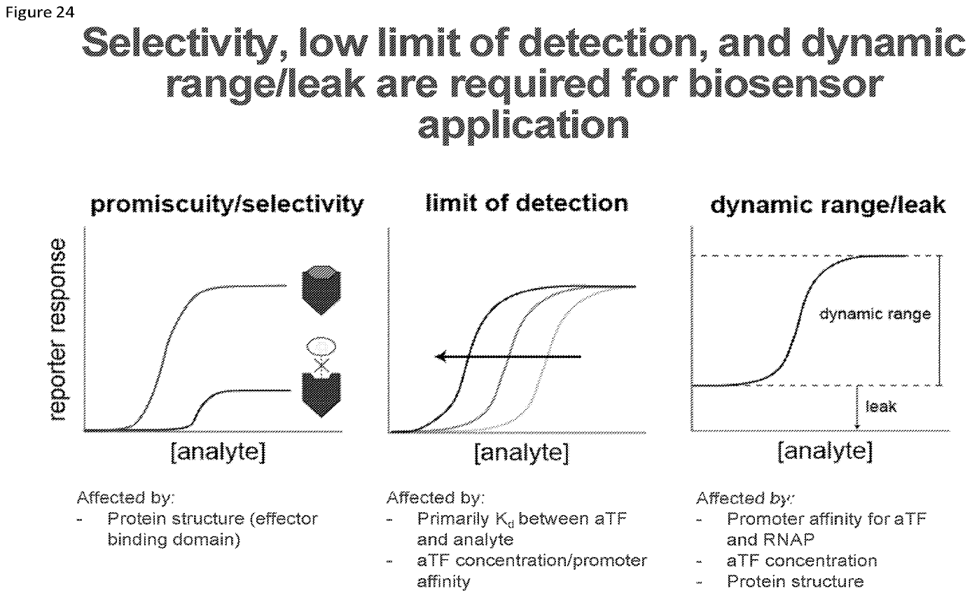

One aspect relates to promiscuity or selectivity. Typically, an aTF that is selective for the analyte of interest and that is not promiscuous for analytes of similar structure or charge is desired. This can be accomplished by engineering, e.g., changing protein structure, often focused on changing the effector binding domain. See FIG. 24.

Another consideration is limit of detection. Most water contaminants have guidelines in the ppb or ppt range, making low limit of detection essential for application. This is most affected by the Kd between aTF and analyte, which is a function of protein structure. A secondary impact is also aTF concentration and the affinity of the aTF for the promoter. See FIG. 24.

Another factor includes dynamic range and leak. These factors are considered for a visually distinguishable positive signal without need for equipment to read output. Both dynamic range and leak are impacted by the promoter architecture and the affinities of the aTF and RNAP for them to initiate transcription. aTF concentration and protein structure are also factors. See FIG. 24.

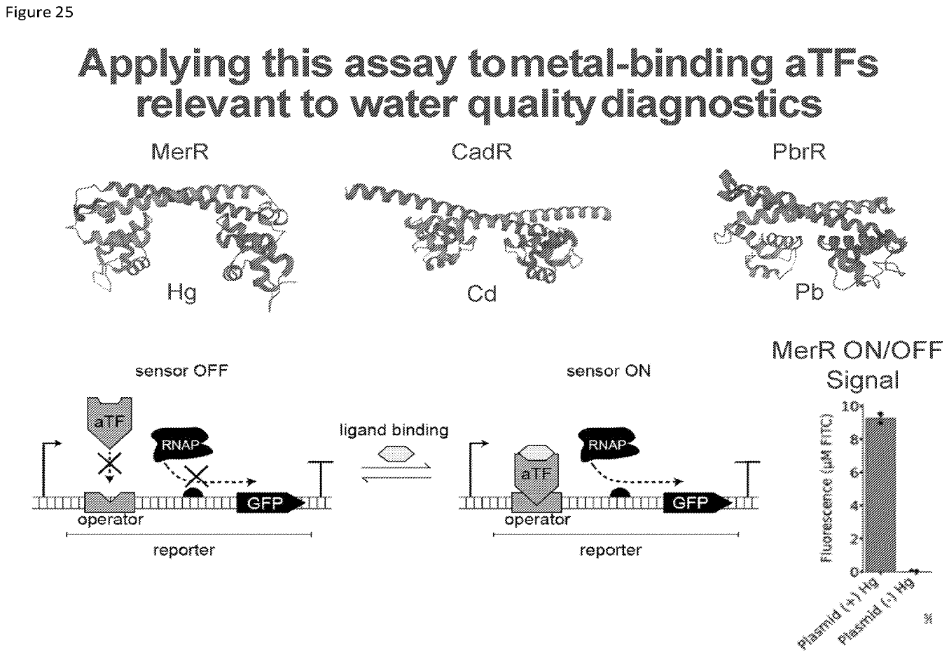

To exemplify this assay, the metalloregulating TFs MerR, which senses mercury, CadR, which senses cadmium, and PbrR, which senses lead are used herein. These sensors are extremely relevant to water quality diagnostics. See FIG. 25.

These TF are also advantageous because their general activator mechanism is known. In the absence of ligand, the TF will not bind to the operator site and the reporter gene (GFP in this example) will not be transcribed. In the presence of ligand, the TF will bind to the operator site, and GFP will be transcribed, making activity easy to measure as fluorescence. Their promoter sequences are also known and can be kept constant throughout the assay.

Cell-free sensing reactions can be constructed by combining aTF DNA (which in this example will be varied in the form of a library), cellular extract, cofactors, reporter DNA, and whatever analytes are of interest to test with the aTF. In some embodiments, an Echo liquid handling robot is used to assemble the cell-free sensing reactions at 1 uL scale in 384-well plates. In each well, a separate aTF variant is expressed against a chosen analyte. See Figure S4.

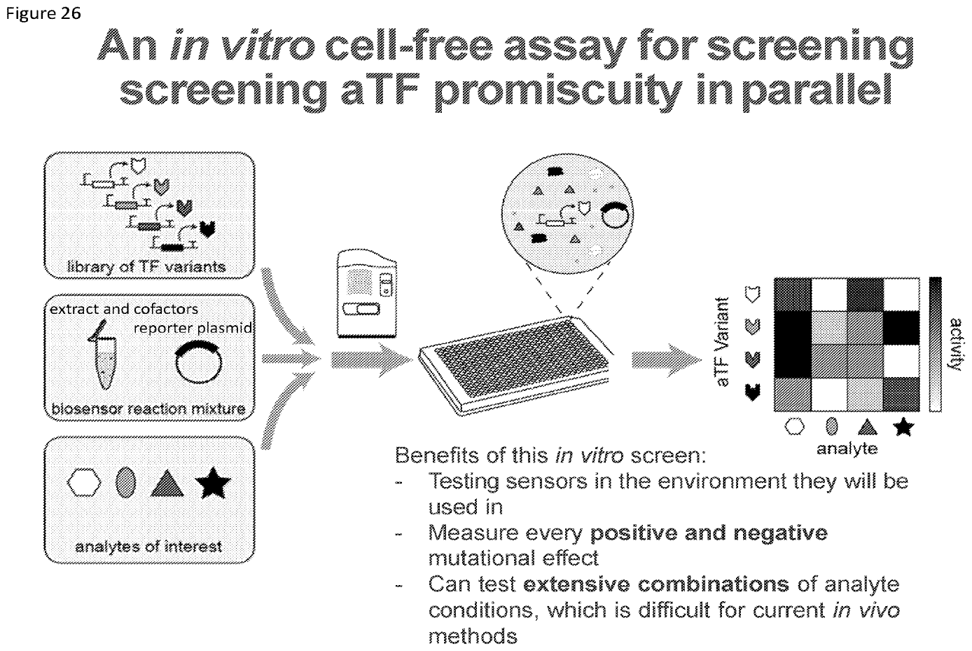

By measuring the sensor outputs in the form of fluorescence, all the aTF variants can be tested against all the analytes and a mutability chart in the form of a heat map can be created (see e.g., See FIG. 26), comparing the relative activity of each aTF for each analyte.

Exemplary benefits of this screen are that all sensors are measured in the same cell-free reaction in which they would be used. Every positive and negative mutational effect can be observed, which is important for probing the sequence-function relationship, and extensive combinations of analyte conditions can be tested, which would be very difficult with current in vivo methods.

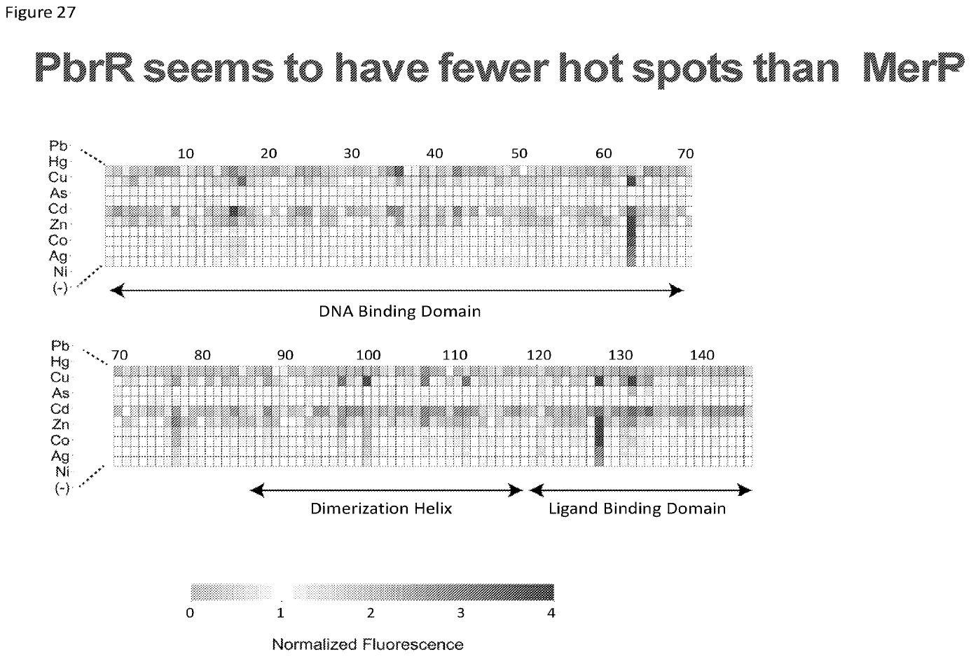

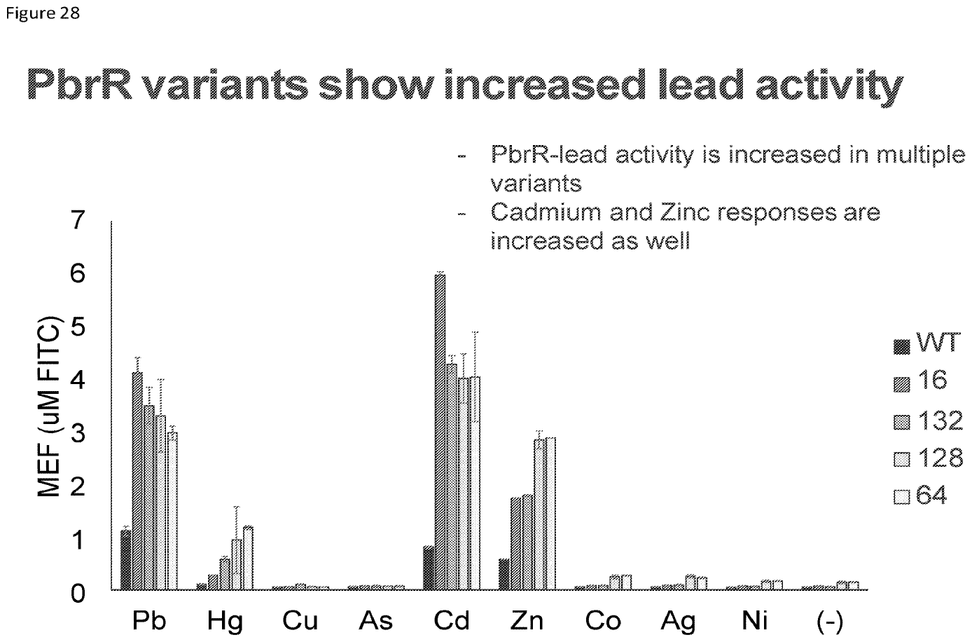

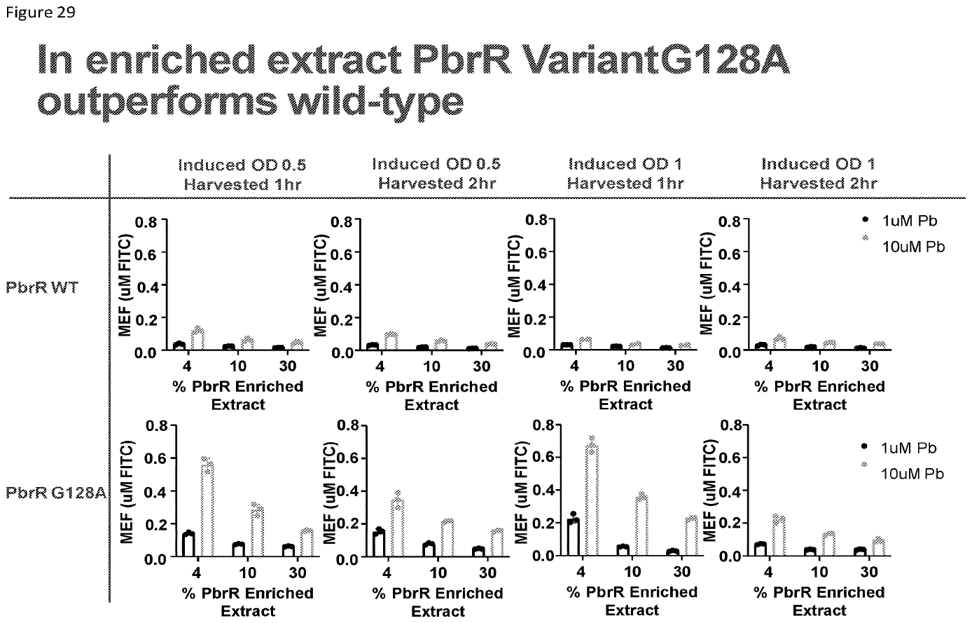

Exemplary data is shown in FIGS. 27 and 28 for various PbrR variants and WT PbrR expressed in vitro with fluorescent reporter. Exemplary data is shown in FIG. 29 for the G128A variant and WT PbrR expressed in vitro with fluorescent reporter.

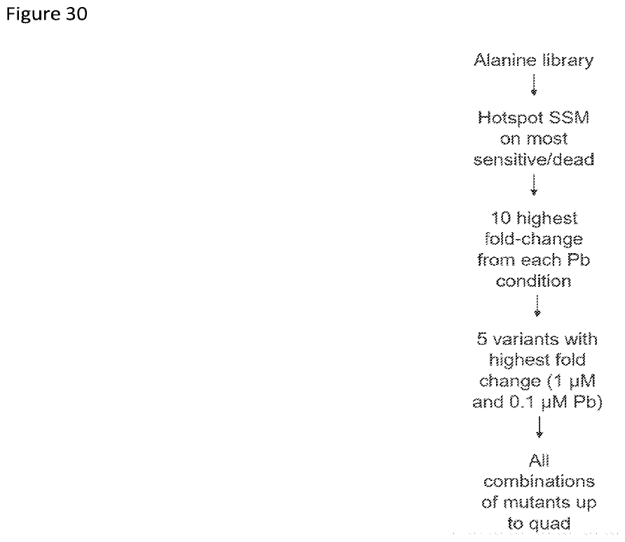

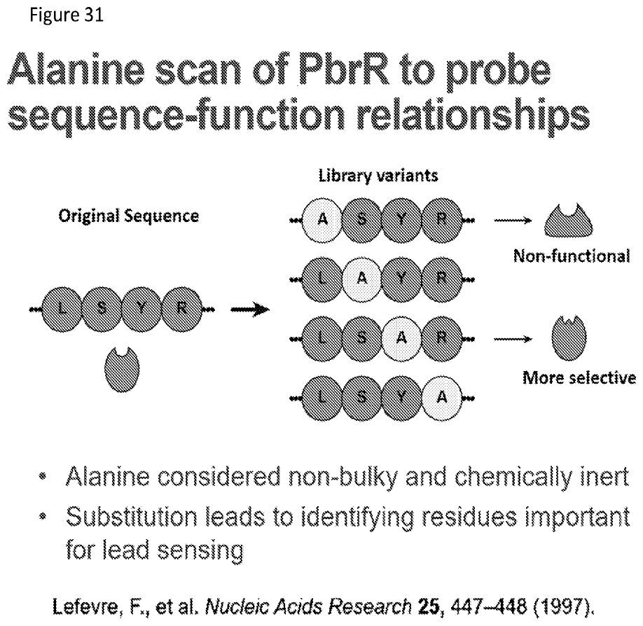

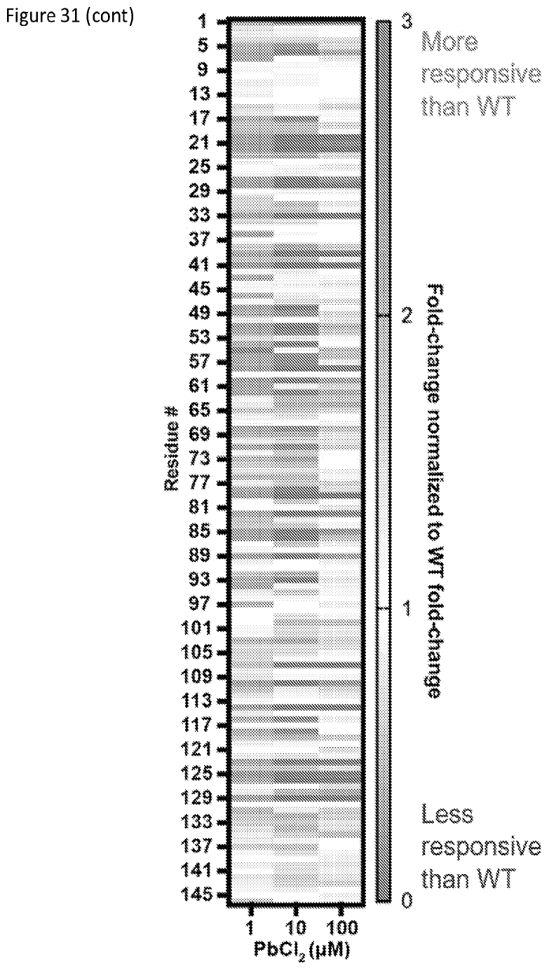

A screen for additional PbrR variants is described with respect to FIGS. 30-40. FIG. 30 depicts a workflow for an alanine scanning screen of PbrR to probe the sequence-function relationship. Alanine is considered non-bulky and chemically inert. Alanine substitution leads to identifying residues important for lead sensing. An alanine site substitution library was made, and then hotspots were iteratively identified, and then site saturation mutagenesis was performed on the identified hotspots. For PbrR, the DNA binding region is approximately at amino acid residues 1-80, the dimerization helix is at approximately residues 80-120, and the effector binding domain is approximately residues 120-145. The alanine library variants were assayed for ability to express a reporter gene in the presence of varying amounts of PbCl₂. The fold-change in the alanine variants was normalized to the WT fold-change. FIG. 31 depicts the results of the initial alanine screening.

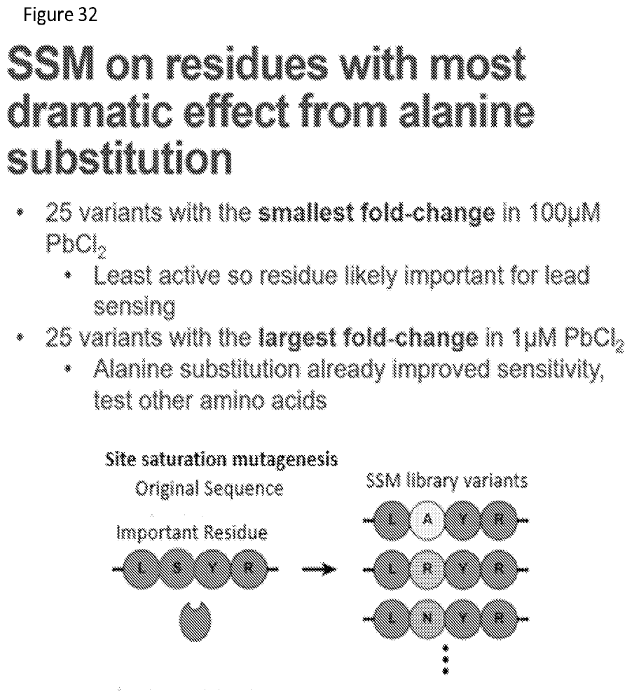

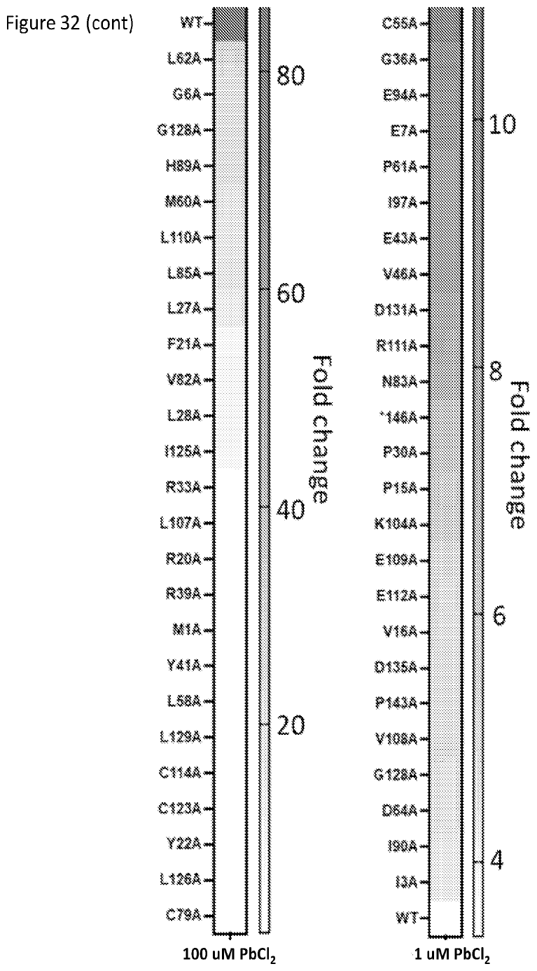

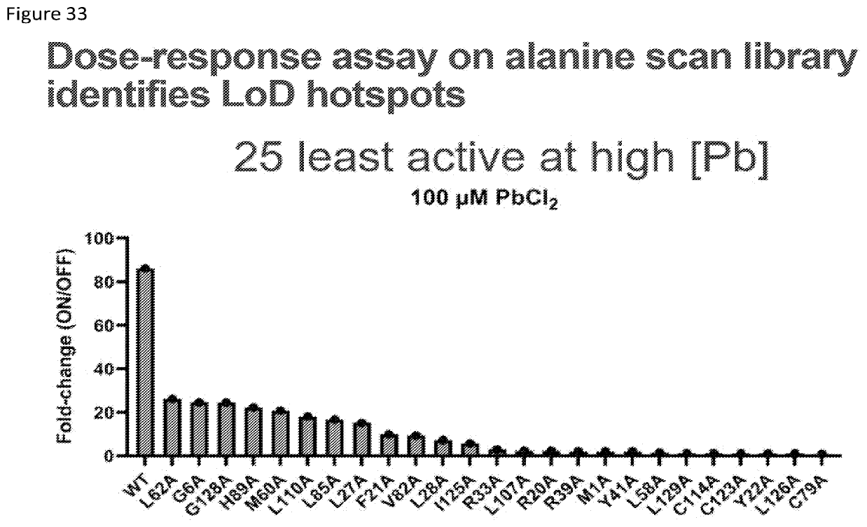

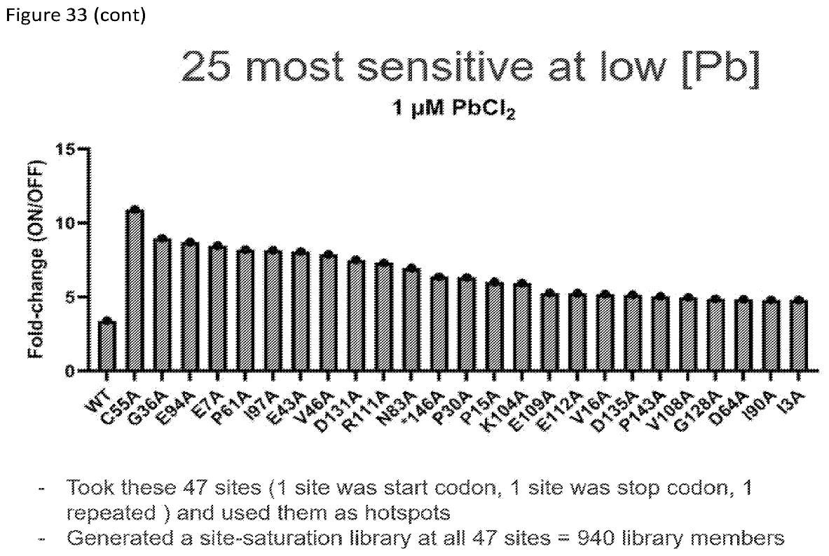

Based on the alanine library screening results shown in FIG. 31, hotspots were chosen and approximately 25 hotspots from the largest fold-change at 1 M PbCl₂, and 25 hotspots from the smallest fold-change in 100 M PbCl₂ were identified for further studies. It was hypothesized that these hotspots are important to function. These approximately 50 total hotspots are depicted in FIGS. 32 and 33. One of the hotspots was a start codon and one was a stop codon, and one was repeated, thus there are a total of 47 hotspots further studied.

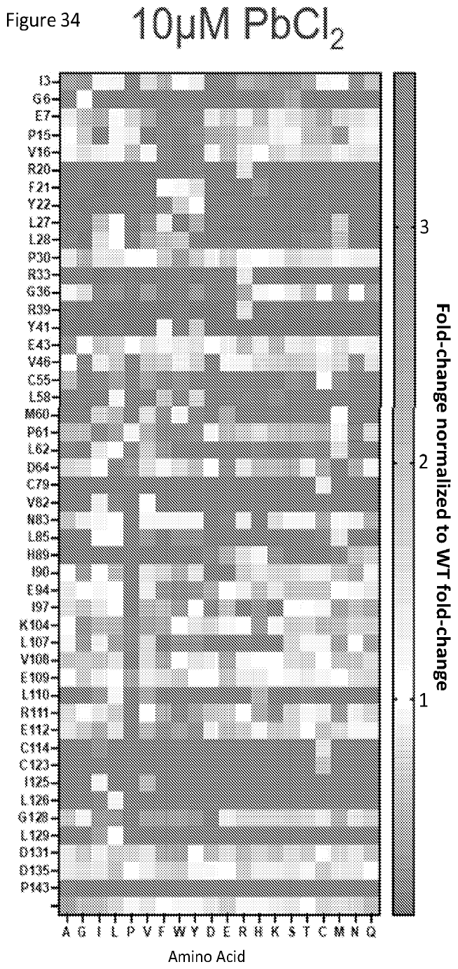

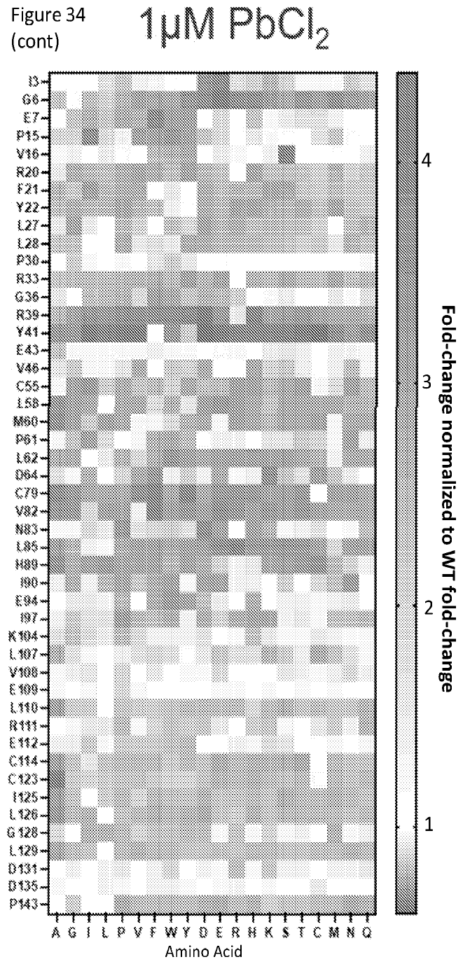

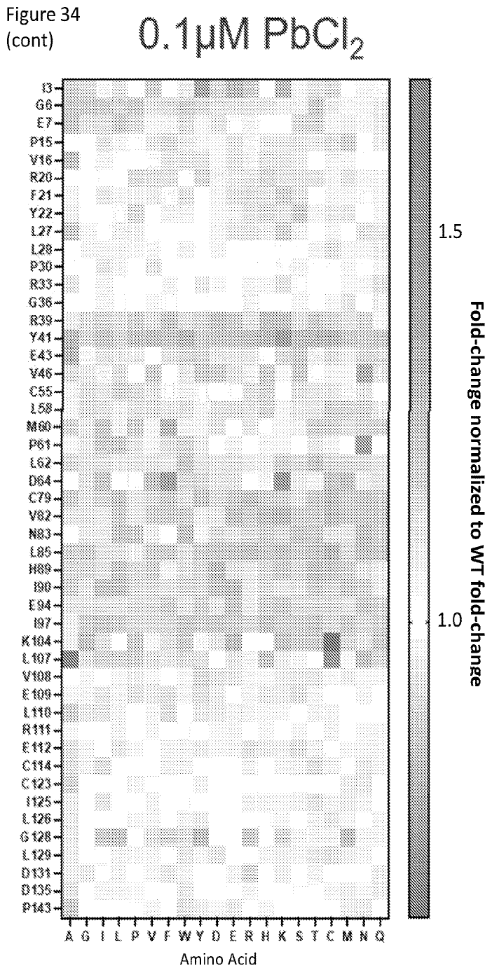

A site saturation mutagenesis (SSM) library was generated at all 47 sites (each of the 20 amino acid variants at each of the 47 sites) for a total of 940 library members. The SSM library was assayed for the ability to express a reporter gene in the presence of varying amounts of PbCl₂. The fold-change in the alanine variants was normalized to the WT fold-change, and FIG. 34 depicts these results. It was determined that variants at some of the sites, 27 and 28, are nonfunctional unless they are the wild type residues (L27 and L28). Residues D64 and G128 showed an increase in sensitivity with some variants from the SSM. Residue L107 increased sensitivity at low concentration of lead but no effect at higher concentrations (high background).

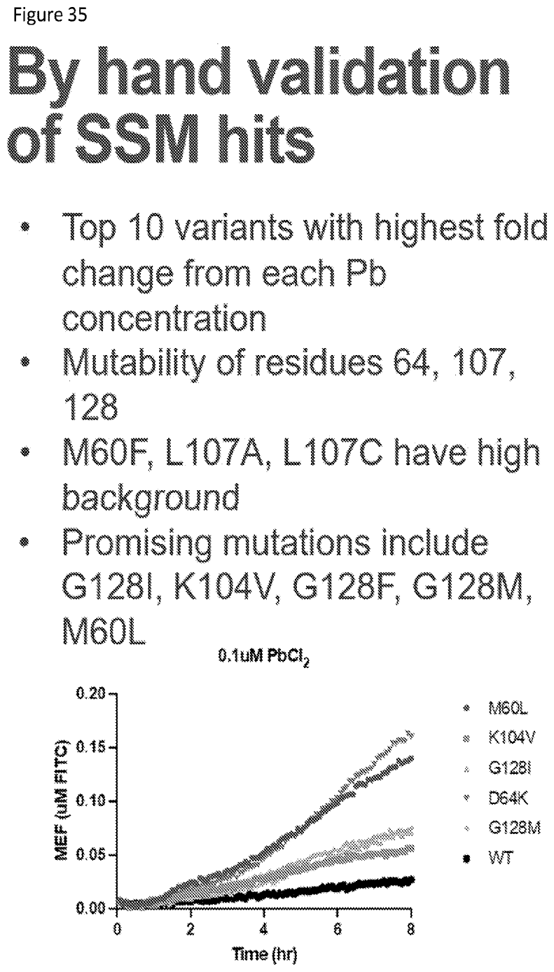

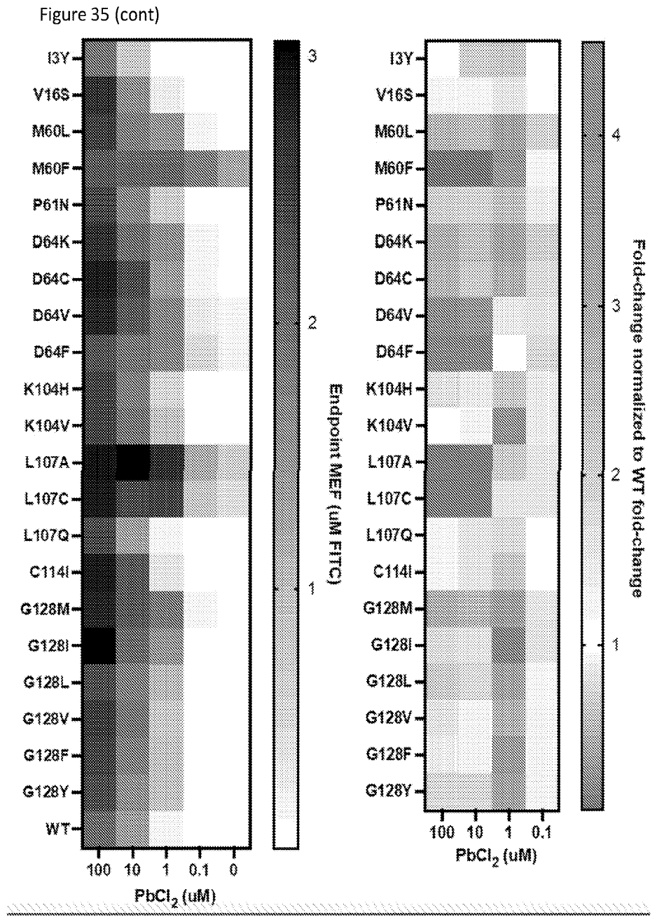

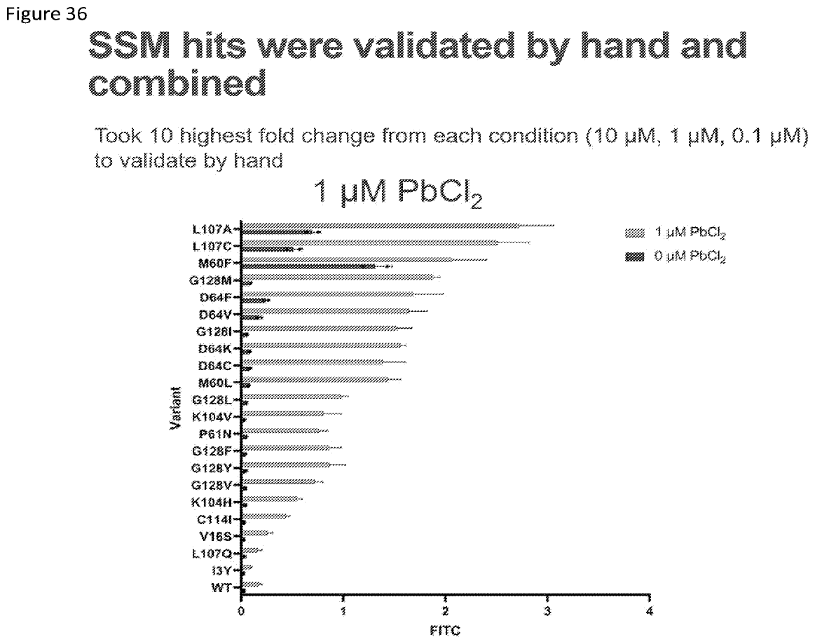

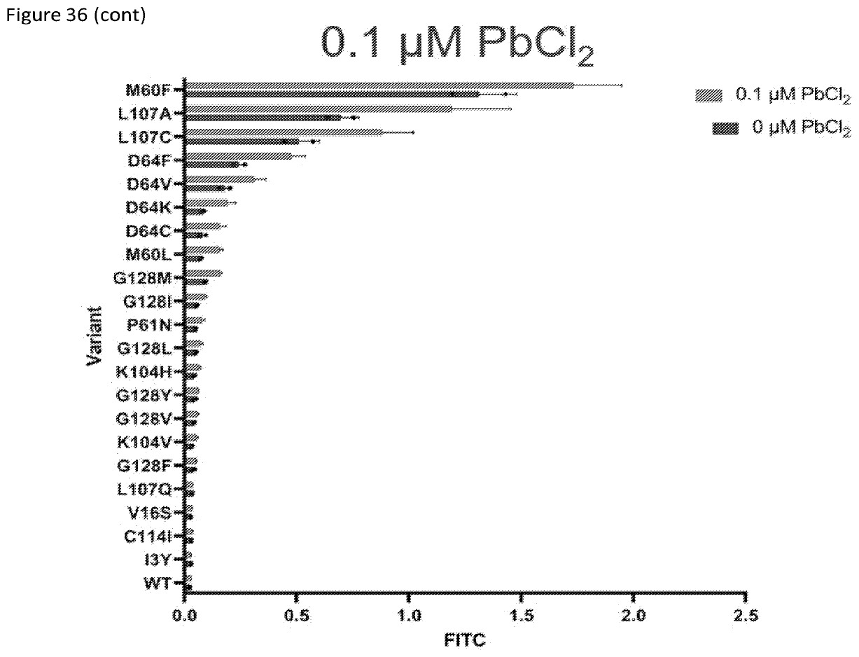

The top 10 variants with the highest fold change from each lead concentration were hand-validated to validate the results from the SSM library screen. FIGS. 35 and 36 shows the results. The M60F variant appeared to cause the PbrR transcription factor to bind the promoter regardless of whether or not the ligand (lead) is present. From these results, it was determined that promising mutations/variants include G128I, K104V, G128F, G128M, and M60L.

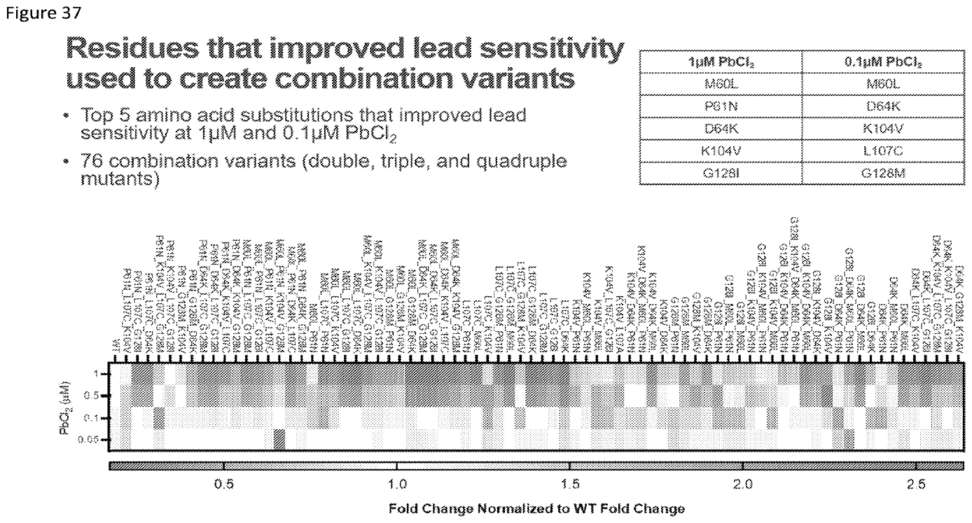

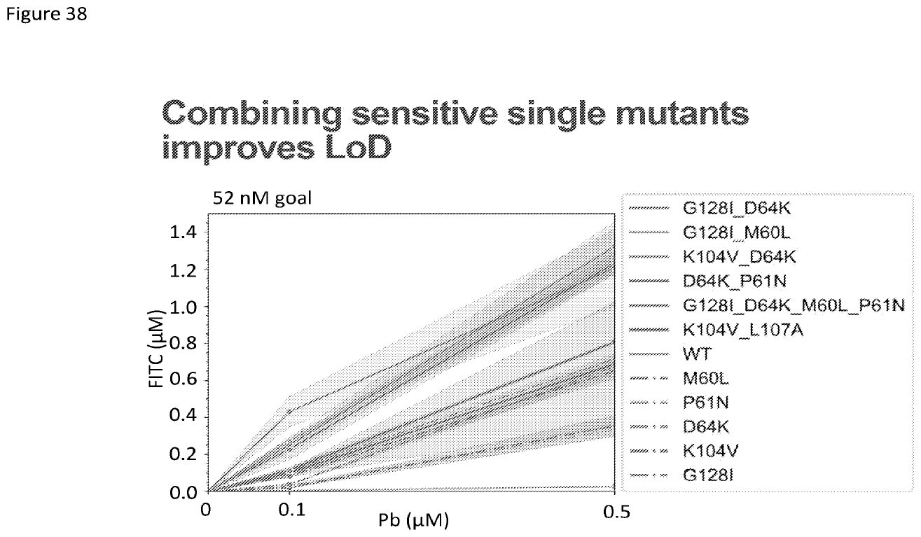

Next residues that improved lead sensitivity were used to create combination variants, and 76 combination variants were made. Specifically, all possible combination variants were made by taking the top 5 amino acids substitutions at unique positions for the two lowest lead concentrations tested from those shown in FIG. 34. The results of shown in FIG. 37. FIG. 38 shows the combination variants that have greater sensitivity than the individual mutations that they are comprised of, e.g., with improved limit of detection (LoD).

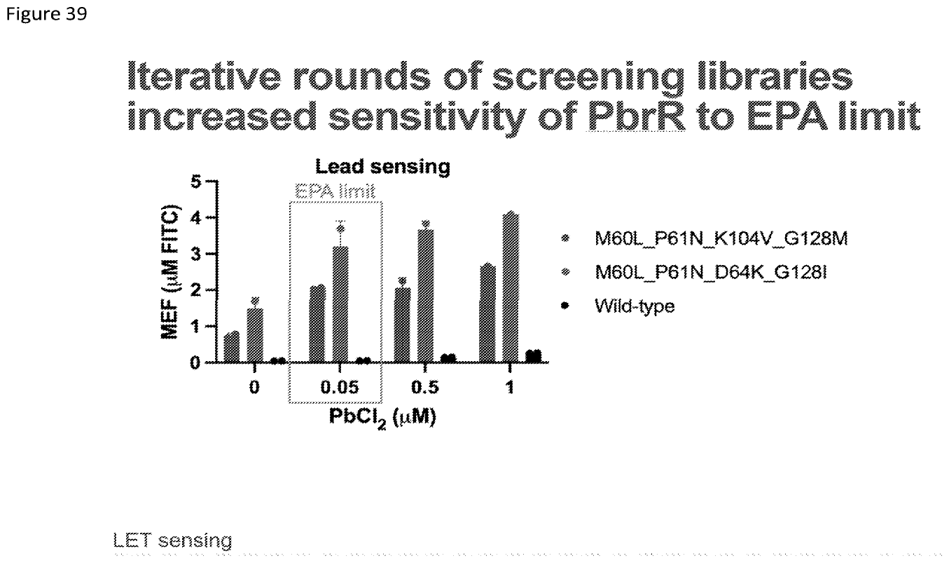

Iterative rounds of screening libraries increased the sensitivity of PbrR variants to the EPA limit for lead (0.05 M PbCl₂). These results and two combination variants are depicted in FIG. 39.

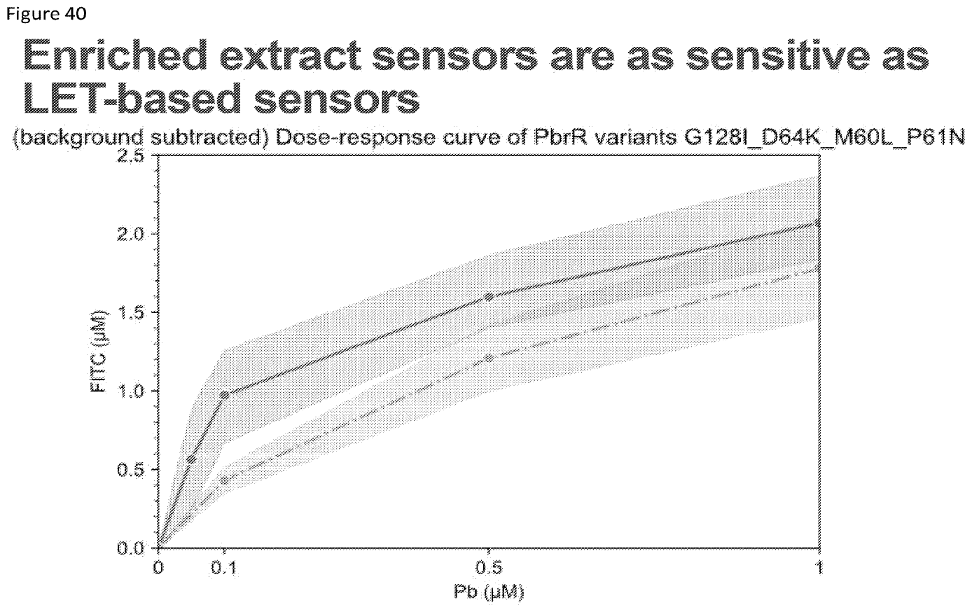

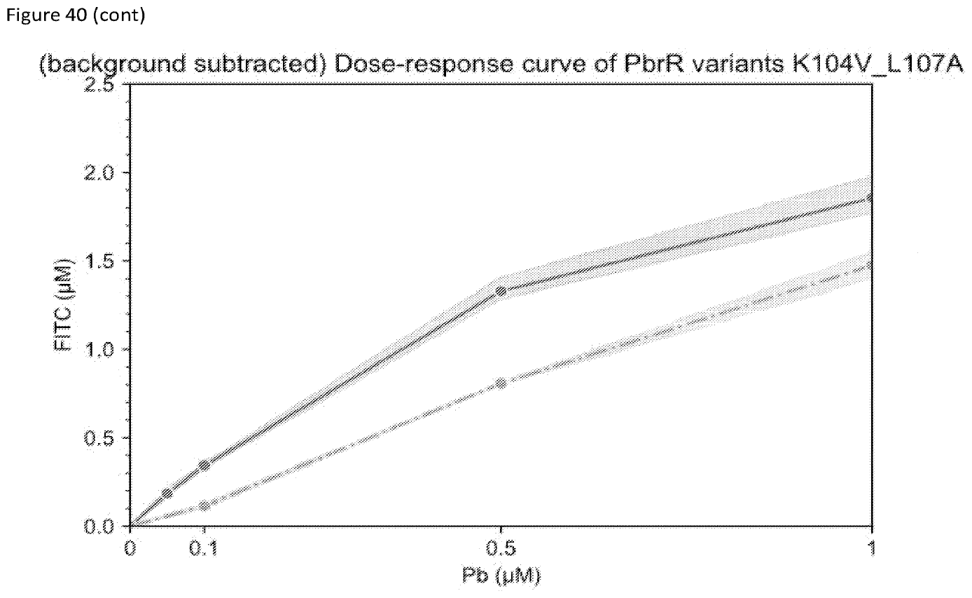

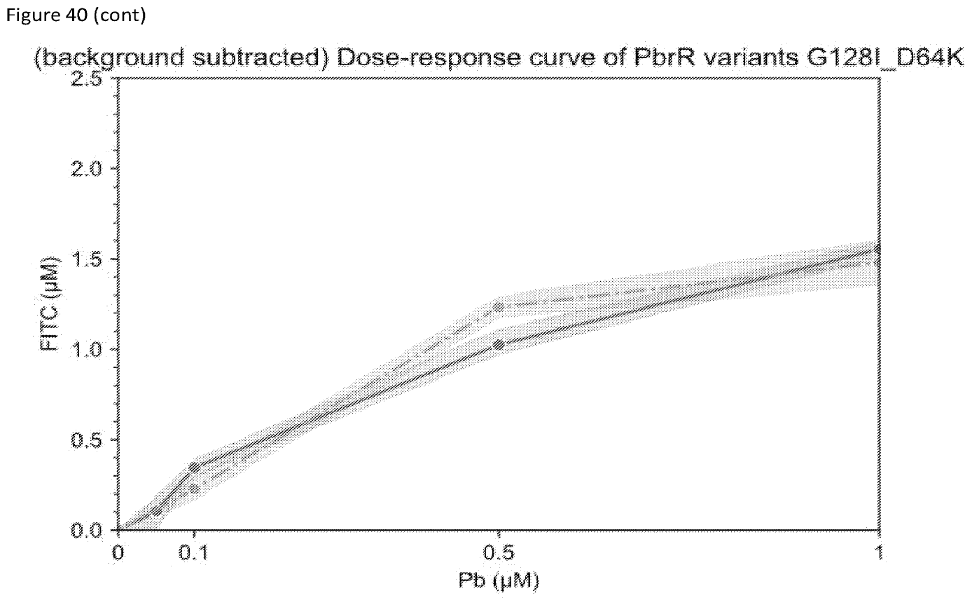

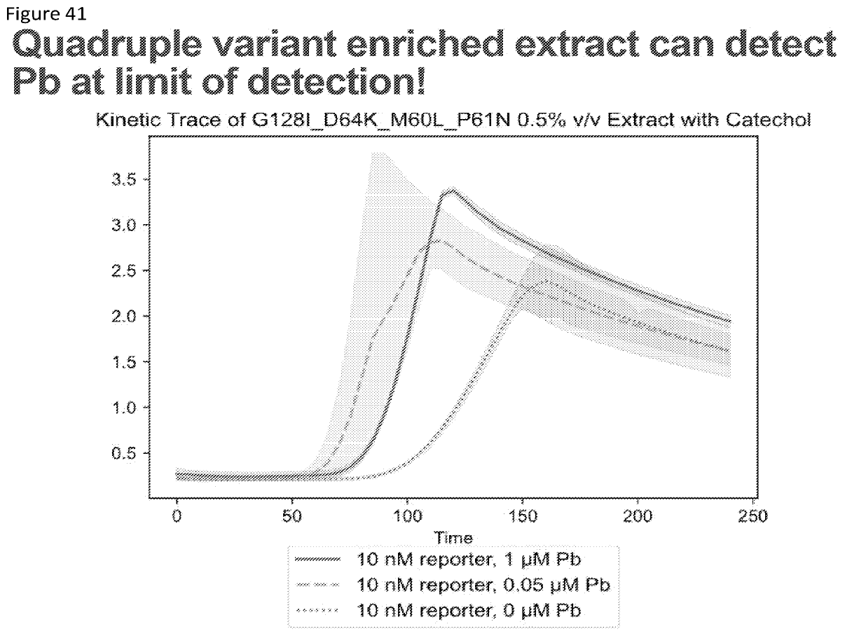

FIG. 40 shows that enriched extract sensors are as sensitive as linear expression template (LET) sensors. Enriched extract is made by pre-expressing the transcription factor in the E. coli strain before lysing to generate the extract. FIG. 41 shows that the quadruple variant G128I, D64K, M60L, and P61N in enriched extract can detect lead at the limit of detection. It was also determined that a catechol reporter can be used for improved sensor output. This works by having the PbrR transcription factor bind to PbrO and drive expression of an enzyme that catalyzes a colorimetric change in catechol.

Cupriavidus metallidurans PbrR (regulator involved in PB(II) resistance); GenBank: ABF12805.1, SEQ ID NO: 47 is shown below:

```
    1 MNIQIGELAK RTACPVVTIR FYEQEGLLPP PGRSRGNFRL YGEEHVERLQ FIRHCRSLDM
  61 PLSDVRTLLS YRKRPDQDCG EVNMLLDEHI RQVESRIGAL LELKHHLVEL REACSGARPA
121 QSCGILQGLS DCVCDTRGTT AHPSD
```

In some embodiments, a PbrR variant comprises one or more of the following (with reference to SEQ ID NO: 47, above): a variant amino acid at position 3, 16, 60, 61, 64, 104, 107, 114, 128, 132. In some embodiments, the variant amino acid is alanine. In some embodiments, the variant at position 3 is alanine or tyrosine. In some embodiments, the variant at position 16 is alanine or serine. In some embodiments, the variant at position 60 is alanine, leucine, or phenylalanine. In some embodiments, the variant at position 61 is alanine or asparagine. In embodiments, the variant at position 64 is alanine, leucine, lysine, cysteine, or phenylalanine. In some embodiments, the variant at position 104 is alanine, valine, or histidine. In some embodiments, the variant at position 107 is alanine, glutamine, or cysteine. In some embodiments, the variant at position 114 is alanine or isoleucine. In some embodiments, the variant at position 128 is alanine, methionine, valine, isoleucine, or phenylalanine. In some embodiments, the variant at position 132 is alanine. In some embodiments, the variant is G128A. In some embodiments, the variant is D64A. In some embodiments, the variant is V16A. In some embodiments, the variant is C132A. In some embodiments, the variant is V16S. In some embodiments, the variant is I3A, or I3Y. In some embodiments, the variant is M60A, M60L, or M60F. In some embodiments, the variant is P61A or P61N. In some embodiments, the variant is D64A, D64L, D64K, D64C, or D64F. In some embodiments, the variant is K104A, K104V, or K104H. In some embodiments, the variant is L107A, L107Q, or L107C. In some embodiments, the variant is C114A or C114I. In some embodiments, the variant is G128A, G128M, G128V, G128I, or G128F. In some embodiments, the variant includes one or more of M60A, M60L, M60F, P61A, P61N, D64A, D64L, D64K, D64C, D64F, K104A, K104V, K104H, L107A, L107Q, L107C, G128A, G128M, G128V, G128I, or G128F.

PbrR WT DNA Sequence (SEQ ID NO: 48):
ATGAACATTCAGATCGGTGAACTGGCGAAACGTACTGCATGTCCAG
TGGTGACCATTCGCTTTTATGAGCAAGAAGGTTTGCTGCCGCCCCC
AGGACGTTCCCGTGGTAATTTCCGCTTATACGGGGAAGAGCATGTT
GAGCGTTTGCAATTTATCCGCCATTGCCGCTCACTTGACATGCCCT
TATCCGATGTCCGCACATTGCTGTCATACCGCAAGCGTCCTGATCA
AGATTGCGGGGAGGTGAACATGCTTCTGGATGAGCATATTCGTCAA
GTAGAGAGTCGTATTGGCGCTCTGTTAGAGTTAAAACATCACTTGG
TCGAGCTGCGCGAAGCATGCTCAGGGGCTCGCCCTGCACAAAGCTG
CGGAATCCTTCAGGGCCTGAGTGATTGTGTTTGCGATACACGTGGC
ACGACAGCACACCCCTCGGAT

While the present example demonstrates novel screening method using the PbrR sequence, variants of the MerR and CadR aTF proteins can also be produced and analyzed using the same method, to identify those that variants provide improved characteristics (e.g., selectivity, limit of detection, dynamic range/leak), as compared to their wild-type counterparts.

MerR WT DNA Sequence (SEQ ID NO: 49):
ATGGAAAATAATTTGGAAAACCTGACCATTGGCGTTTTTGCCAAGG
CGGCCGGGGTCAACGTGGAGACAATCCGCTTCTATCAGCGCAAGGG
CCTGTTGCGGGAACCGGACAAGCCTTACGGCAGCATCCGCCGCTAT
GGGGAGGCGGACGTGGTTCGGGTGAAATTCGTGAAATCGGCACAGC
GGCTGGGGTTCAGTCTGGACGAGATTGCCGAGCTGTTGCGGCTCGA
CGATGGCACCCACTGCGAGGAGGCCAGCAGCCTGGCCGAACACAAG
CTCAAGGACGTGCGCGAGAAGATGGCCGACTTGGCGCGCATGGAAA
CCGTGCTGTCTGAACTCGTGTGCGCCTGCCATGCACGAAAGGGGAA
TGTTTCCTGCCCGTTGATCGCGTCACTACAGGGCGAAGCAGGCCTG
GCAAGGTCAGCTATGCCT
MerR Protein (NCBI Reference Sequence:
WP_000429836.1) (SEQ ID NO: 50):
MENNLENLTIGVFAKAAGVNVETIRFYQRKGLLREPDKPYGSIRRY
GEADVVRVKFVKSAQRLGFSLDEIAELLRLDDGTHCEEASSLAEHK
LKDVREKMADLARMETVLSELVCACHARKGNVSCPLIASLQGEAGL
ARSAMP
CadR WT DNA Sequence (SEQ ID NO: 51):
ATGAAAATCGGTGAACTTGCTAAAGCCACGGACTGTGCGGTTGAAA
CTATCCGTTACTACGAGCGTGAACAGTTGCTGCCAGAGCCTGCCCG
TTCAGACGGTAATTATCGCTTATACACGCAGGCACATGTGGAACGT
CTGACGTTTATTCGCAACTGCCGTACATTGGATATGACTCTTGATG
AAATCCGTTCTCTGTTGCGTTTGCGTGATTCGCCGGATGATAGCTG
CGGGTCCGTGAACGCGTTAATTGATGAACATATTGAGCATGTGCAA
GCTCGTATCGATGGACTGGTTGCGCTTCAGGAGCAACTGGTAGAAC
TTCGTCGTCGCTGCAATGCCCAAGGTGCTGAGTGTGCCATCTTACA
GCAATTGGAGACGAACGGTGCTGTTTCGGTCCCCGAAACAGAACAC
TCCCATGTGGGTCGCTCTCACGGCCAC
Protein (NCBI Reference Sequence:
WP_015272183.1) (SEQ ID NO: 52):
MKIGELAKATDCAVETIRYYEREQLLPEPARSDGNYRLYTQAHVER
LTFIRNCRTLDMTLDEIRSLLRLRDSPDDSCGSVNALIDEHIEHVQ
ARIDGLVALQEQLVELRRRCNAQGAECAILQQLETNGAVSVPETEH
SHVGRSHGH

### EXEMPLARY EMBODIMENTS

The following are exemplary, non-limiting embodiments of the methods and compositions disclosed herein.
1. A method of detecting a molecule in a sample, the method comprising: (a) obtaining a biological or environmental sample which may or may not contain the target molecule; (b) concentrating and/or solubilizing the molecule in the sample if necessary; (c) contacting the molecule to a cell-free protein synthesis reaction, wherein the contacting occurs under conditions that directly or indirectly lead to a visual, electronic, or optical output in the presence of the target molecule but not in the absence of the target molecule as an indication that the target molecule is present in the sample.
2. The method of embodiment 1, wherein a transcription factor responsive to the molecule (e.g., a metabolite) enables the synthesis of a reporter protein with a visual, electronic, or optical output in the presence of the target molecule but not in the absence of the target molecule.
3. The method of embodiment 2, wherein the transcription factor is synthesized in situ in the same pot as the reporter protein off plasmid DNA.
4. The method of embodiment 2, wherein the transcription factor is synthesized in situ in the same pot as the reporter protein off linear DNA expression templates.
5. The method of embodiment 2, wherein the transcription factor is overexpressed in the host strain prior to making the extract for cell-free protein synthesis.
6. The method of embodiment 2, wherein the transcription factor is overexpressed in the host strain prior to making the extract for cell-free protein synthesis, and this transcription factor enriched extract is combinatorially mixed in with a blank extract that is not enriched for any specific protein to yield the final extract for cell-free protein synthesis of the reporter.
7. The method of embodiment 2-6, wherein the transcription factor is identified by screening a library of unique, engineered sequences that are expressed in vitro in cell-free biosensing reactions.
8. The method of embodiment 7, wherein the library is comprised of saturation mutagenesis sequences. For each amino acid position of interest, every canonical amino acid sequence is individually substituted in (saturation mutagenesis).
9. The method of embodiment 8, wherein the base transcription factor is bioinformatically mined from a bacterial host genome.
10. The method of embodiment 8, wherein the template transcription factor is a chosen sequence variant (iterative saturation mutagenesis).
11. The method of embodiment 10, wherein subsequent identified amino acid positions of interests are carried through.
12. The method of embodiment 1, wherein the molecule that is sensed is lead.
13. Novel protein sequences for a lead sensing protein based on PbrR from Cupriavidus metallidurans (GenBank: ABF12805.1) wherein residue 64 is an alanine, leucine, lysine, cysteine, phenylalanine, or any other non-native amino acid.
14. Novel protein sequences for a lead sensing protein based on PbrR from Cupriavidus metallidurans (GenBank: ABF12805.1, SEQ ID NO: 47) wherein residue 128 is an alanine, methionine, valine, isoleucine, phenylalanine, or any other, or any other non-native amino acid.
15. Novel protein sequences for a lead sensing protein based on PbrR from Cupriavidus metallidurans (GenBank: ABF12805.1, SEQ ID NO: 47) wherein residue 16 is an alanine, serine, or any other non-native amino acid.
16. Novel protein sequences for a lead sensing protein based on PbrR from Cupriavidus metallidurans (GenBank: ABF12805.1, SEQ ID NO: 47) wherein residue 132 is an alanine, or any other non-native amino acid.
17. Novel protein sequences for a lead sensing protein based on PbrR from Cupriavidus metallidurans (GenBank: ABF12805.1, SEQ ID NO: 47) wherein residue 3 is an alanine, tyrosine, or any other non-native amino acid.
18. Novel protein sequences for a lead sensing protein based on PbrR from Cupriavidus metallidurans (GenBank: ABF12805.1, SEQ ID NO: 47) wherein residue 104 is an alanine, valine, or any other non-native amino acid.
19. Novel protein sequences for a lead sensing protein based on PbrR from Cupriavidus metallidurans (GenBank: ABF12805.1, SEQ ID NO: 47) wherein residue 107 is an alanine, glutamine, cysteine, or any other non-native amino acid.
20. Novel protein sequences for a lead sensing protein based on PbrR from Cupriavidus metallidurans (GenBank: ABF12805.1, SEQ ID NO: 47) wherein residue 114 is an alanine, isoleucine, or any other non-native amino acid.
21. Novel protein sequences for a lead sensing protein based on PbrR from Cupriavidus metallidurans (GenBank: ABF12805.1, SEQ ID NO: 47) wherein residue 61 is an alanine, asparagine, or any other non-native amino acid.
22. Novel protein sequences for a lead sensing protein based on PbrR from Cupriavidus metallidurans (GenBank: ABF12805.1, SEQ ID NO: 47) wherein residue 60 is an alanine, leucine, phenylalanine or any other non-native amino acid.
23. Novel protein sequences for a lead sensing protein based on PbrR from Cupriavidus metallidurans (GenBank: ABF12805.1, SEQ ID NO: 47) wherein residue 104 is an alanine, valine, histidine, or any other non-native amino acid.

In the context of the exemplary embodiments, “non-native amino acid” refers to a different amino acid than the one at the recited position of SEQ ID NO: 47.

### REFERENCES

U.S. Pat. No. 5,478,730 16 Dec. 1992 26 Dec. 1995 Institute of Protein Research Method of preparing polypeptides in cell-free translation system
U.S. Pat. No. 5,556,769 23 Aug. 1994 17 Sep. 1996 Coupled replication-translation methods and kits for protein synthesis
U.S. Pat. No. 5,665,563 13 Feb. 1995 9 Sep. 1997 Promega Corporation Coupled transcription and translation in eukaryotic cell-free extract
U.S. Pat. No. 6,168,931 17 Mar. 1999 2 Jan. 2001 The Board of Trustees of the Leland Stanford Junior University Enhanced in vitro synthesis of biological macromolecules using a novel ATP regeneration system
U.S. Pat. No. 6,518,058 23 Apr. 2001 11 Feb. 2003 Roche Diagnostics GmbH Method of preparing polypeptides in cell-free system and device for its realization
U.S. Pat. No. 6,783,957 21 Jan. 2003 31 Aug. 2004 Roche Diagnostics GmbH Method for synthesis of polypeptides in cell-free systems
U.S. Pat. No. 6,869,774 28 Aug. 2001 22 Mar. 2005 Yaeta Endo Methods of synthesizing cell-free protein U.S. Pat. No. 6,994,986 7 Sep. 2001 7 Feb. 2006 The Board of Trustees of the Leland Stanford University In vitro synthesis of polypeptides by optimizing amino acid metabolism
U.S. Pat. No. 7,118,883 23 Oct. 2001 10 Oct. 2006 Post Genome Institute Co., Ltd. Process for producing peptides by using in vitro transcription/translation system
U.S. Pat. No. 7,189,528 10 Dec. 2002 13 Mar. 2007 Shimadzu Corporation Extract solution for cell-free protein synthesis, method for cell-free protein synthesis using same and method for preparation of the extract solution
U.S. Pat. No. 7,338,789 18 Aug. 2003 4 Mar. 2008 The Board of Trustees of the Leland Stanford Junior University Methods of in vitro protein synthesis
U.S. Pat. No. 7,387,884 7 Jan. 2004 17 Jun. 2008 Shimadzu Corporation Yeast extract solution for cellfree protein synthesis, method for preparation thereof and method for cell-free protein synthesis using same
U.S. Pat. No. 7,399,610 12 Oct. 2005 15 Jul. 2008 Shimadzu Corporation/Institute of Protein Research Method for cell-free protein synthesis using extract solution derived from insect cell
Pardee and Collins. Harvard College and Boston University. Paper-based synthetic gene networks. US20160312312A1.
Jewett, Hodgman, Gan. Northwestern University. Methods for cell-free protein synthesis. U.S. Pat. No. 9,528,137B2.
Jewett and Karim. Northwestern University. Cell-Free Protein Synthesis Driven Metabolic Engineering. US20160362708A1.
Wen, K. Y., et al., A Cell-Free Biosensor for Detecting Quorum Sensing Molecules in P. aeruginosa-Infected Respiratory Samples. ACS Synthetic Biology, 2017. 6(12): p. 2293-2301.
Kwon, Y. C. and M. C. Jewett, High-throughput preparation methods of crude extract for robust cell-free protein synthesis. Scientific Reports, 2015. 5: p. 8663.
Sun, Z. Z., et al., Protocols for Implementing an Escherichia coli Based TX-TL Cell-Free Expression System for Synthetic Biology. 2013(79): p. e50762.
Pardee, K., et al. “Paper-Based Synthetic Gene Networks.” Cell 159(4): 940-954.
Jia, K., et al., A lower limit of detection for atrazine was obtained using bioluminescent reporter bacteria via a lower incubation temperature. Ecotoxicology and Environmental Safety, 2012. 84: p. 221-226.
Hua, A., et al., Development of a bacterial bioassay for atrazine and cyanuric acid detection. Frontiers in Microbiology, 2015. 6: p. 211.
Garcia-González, V., et al., Regulation of the Pseudomonas sp. Strain ADP Cyanuric Acid Degradation Operon. Journal of Bacteriology, 2005. 187(1): p. 155-167.
Beabout, K., et al., Optimization of Heavy Metal Sensors Based on Transcription Factors and Cell-Free Expression Systems. ACS Synthetic Biology, 2021 10 (11), 3040-3054. DOI: 10.1021/acssynbio.1c00331
Acevedo-Rocha, C., et al., Iterative saturation mutagenesis: a powerful approach to engineer proteins by systematically simulating Darwinian evolution. Methods Mol Biol., 2014; 1179:103-28. doi: 10.1007/978-1-4939-1053-3_7. PMID: 25055773.

### Additional Exemplary Embodiments

Embodiment 1. A composition, kit, or system for detecting a target molecule (e.g., a metabolite, a chemical compound, a nucleic acid) in a sample, the composition, kit, or system comprising one or more of the following components: cellular transcription and translational machinery provided in a lysate from an engineered bacterial strain, or purified separately and reconstituted to defined concentrations, a biosensor molecule that modulates the expression of a target DNA sequence in a DNA transcription template, wherein the biosensor molecule comprises an allosteric transcription factor comprising SEQ ID NO: 47 or a variant thereof; a DNA transcription template whose expression is configured to be regulated by the biosensor molecule, and encoding a reporter molecule (e.g., a reporter protein or RNA molecule).

Embodiment 2. The composition, kit, or system of embodiment 1, wherein the lysate comprises metabolism from the host strain that provides one or more of the following: (i) energy (e.g., ATP-based regeneration systems or non-phosphate based energy); (ii) cofactor regeneration; (iii) enzymes or transcriptional regulators used for cell-free sensing; or (iv) any combination thereof and optionally exogenously supplied cell-free protein synthesis reagents, including enzyme substrates and cofactors.

Embodiment 3. The composition, kit, or system of embodiment 1, wherein the biosensor molecule is responsive to the target molecule, wherein the biosensor molecule enables the expression of the reporter molecule in the presence of the target molecule but not in the absence of the target molecule.

Embodiment 4. The composition, kit, or system of embodiment 3, wherein the allosteric transcription factor consists of SEQ ID NO: 47.

Embodiment 5. The composition, kit, or system of embodiment 1, wherein the allosteric transcription factor comprises a variant of SEQ ID NO: 47, wherein the variant comprises a substitution mutation at one or more of amino acid positions 3, 16, 60, 61, 64, 104, 107, 114, 128, or 132.

Embodiment 6. The composition, kit, or system of embodiment 5, wherein the variant comprises a substitution mutation at one or more of amino acid positions 60, 61, 64, 104, 107, or 128.

Embodiment 7. The composition, kit, or system of embodiment 6, wherein the variant consists of one or more of M60A, M60L, M60F, P61A, P61N, D64A, D64L, D64K, D64C, D64F, K104A, K104V, K104H, L107A, L107Q, L107C, G128A, G128M, G128V, G128I, or G128F.

Embodiment 8. The composition, kit, or system of any of the previous claims, wherein the biosensor molecule is overexpressed in the host strain prior to making the extract for cell-free protein synthesis.

Embodiment 9. The composition, kit, or system of embodiment 1, wherein the reporter molecule is a fluorescent protein such as green fluorescent protein (GFP), red fluorescent protein (RFP), blue fluorescent protein (BFP), or any derivatives thereof.

Embodiment 10. The composition, kit, or system of embodiment 1, wherein the reporter molecule is an enzyme that produces a visible signal, such as catechol 2,3-dioxygenase (C23DO), beta-galactosidase (LacZ), or glucuronidase (GusA).

Embodiment 11. A method for detecting a target molecule (e.g., a metabolite, a chemical compound, a nucleic acid) in a sample using the composition, kit, or system of any of embodiments 1-10, wherein the target molecule comprises lead.

Embodiment 12. The method of any of the previous embodiments, wherein the sample comprises an environmental sample, or a biological sample (e.g., from a subject).

Embodiment 13. The method of embodiment 12, wherein the environmental sample comprises a water sample or a soil sample.

Embodiment 14. A method for detecting a target molecule in a sample, the method comprising using one or more of the components of the composition, kit, or system of any of the previous embodiments, wherein the presence of the target molecule increases the rate of transcription and translation of the reporter molecule from the DNA transcription template.

Embodiment 15. A composition, kit, or system of detecting a target molecule comprising a chemical compound in a sample, the composition, kit, or system comprising (a) a biosensor molecule that modulates the expression of a target DNA sequence in a DNA transcription template, the biosensor molecule comprising SEQ ID NO: 47 or a variant thereof, and (b) one or more of the following components: (i). the cellular transcription and translational machinery provided in a lysate from an engineered bacterial strain, or purified separately and reconstituted to defined concentrations; (ii). a DNA transcription template whose expression is configured to be modulated by the biosensor molecule, and encoding a target DNA sequence, wherein the target DNA sequence comprises a reporter molecule.

Embodiment 16. The composition, kit, or system of any of the previous embodiments wherein one or more of the components are lyophilized.

Embodiment 17. A method of detecting a target molecule comprising a chemical compound in a biological or environmental sample, the method comprising: (a) obtaining a biological or environmental sample which may or may not contain the target molecule and optionally concentrating and/or solubilizing the target molecule in the sample if necessary; (b) adding the sample and/or the optionally concentrated and/or solubilized target molecule in the sample to a cell-free protein synthesis (CFPS) reaction, wherein if the target molecule is present in the sample then an output is generated (e.g., a visual, electronic, or optical output); wherein the output is generated via steps that include: (i) the target molecule inducing expression from a DNA transcription template encoding a reporter molecule via a biosensor molecule, the biosensor molecule comprising SEQ ID NO: 47 or a variant thereof, and (ii) the reporter molecule generating an output either directly or indirectly; (iii) detecting the presence or absence of the target molecule based on the reporter molecule output.

Embodiment 18. The method of embodiment 17, wherein the cell-free protein synthesis reaction comprises: (a) a cell extract from a host strain that (i) provides energy; (ii) provides cofactor regeneration; (iii) provides enzymes used for cell-free sensing of the target molecule; or (iv) any combination thereof, and (b) exogenous supplied cell-free protein synthesis reagents not present in the cell extract that comprise at least one transcription template and a polymerase.

Embodiment 19. An engineered polypeptide comprising a variant of SEQ ID NO: 47, wherein the variant comprises one or more substitution mutations selected from M60A, M60L, M60F, P61A, P61N, D64A, D64L, D64K, D64C, D64F, K104A, K104V, K104H, L107A, L107Q, L107C, G128A, G128M, G128V, G128I, and G128F.

Embodiment 20. A cell-free transcription and translation system composition comprising the engineered polypeptide of embodiment 19.

Embodiment 21. The cell free transcription and translation system of embodiment 20, wherein the system is lyophilized.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element In the or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A composition, kit, or system for detecting a target molecule (e.g., a metabolite, a chemical compound, a nucleic acid) in a sample, the composition, kit, or system comprising one or more of the following components:
(a) cellular transcription and translational machinery provided in a lysate from an engineered bacterial strain, or purified separately and reconstituted to defined concentrations,
(b) a biosensor molecule that modulates the expression of a target DNA sequence in a DNA transcription template, wherein the biosensor molecule comprises an allosteric transcription factor comprising SEQ ID NO: 47 or a variant thereof,
(c) a DNA transcription template whose expression is configured to be regulated by the biosensor molecule, and encoding a reporter molecule (e.g., a reporter protein or RNA molecule).

2. The composition, kit, or system of claim 1, wherein the lysate comprises metabolism from the host strain that provides one or more of the following: (i) energy (e.g., ATP-based regeneration systems or non-phosphate based energy); (ii) cofactor regeneration; (iii) enzymes or transcriptional regulators used for cell-free sensing; or (iv) any combination thereof and optionally exogenously supplied cell-free protein synthesis reagents, including enzyme substrates and cofactors.

3. The composition, kit, or system of claim 1, wherein the biosensor molecule is responsive to the target molecule, wherein the biosensor molecule enables the expression of the reporter molecule in the presence of the target molecule but not in the absence of the target molecule.

4. The composition, kit, or system of claim 3, wherein the allosteric transcription factor consists of SEQ ID NO: 47.

5. The composition, kit, or system of claim 1, wherein the allosteric transcription factor comprises a variant of SEQ ID NO: 47, wherein the variant comprises a substitution mutation at one or more of amino acid positions 3, 16, 60, 61, 64, 104, 107, 114, 128, or 132.

6. The composition, kit, or system of claim 5, wherein the variant comprises a substitution mutation at one or more of amino acid positions 60, 61, 64, 104, 107, or 128.

7. The composition, kit, or system of claim 6, wherein the variant consists of one or more of M60A, M60L, M60F, P61A, P61N, D64A, D64L, D64K, D64C, D64F, K104A, K104V, K104H, L107A, L107Q, L107C, G128A, G128M, G128V, G128I, or G128F.

8. The composition, kit, or system of claim 1, wherein the biosensor molecule is overexpressed in the host strain prior to making the extract for cell-free protein synthesis.

9. The composition, kit, or system of claim 1, wherein the reporter molecule is a fluorescent protein such as green fluorescent protein (GFP), red fluorescent protein (RFP), blue fluorescent protein (BFP), or any derivatives thereof.

10. The composition, kit, or system of claim 1, wherein the reporter molecule is an enzyme that produces a visible signal, such as catechol 2,3-dioxygenase (C23DO), beta-galactosidase (LacZ), or glucuronidase (GusA).

11. A method for detecting a target molecule (e.g., a metabolite, a chemical compound, a nucleic acid) in a sample using the composition, kit, or system of claim 1, wherein the target molecule comprises lead.

12. The method of claim 11, wherein the sample comprises an environmental sample, or a biological sample (e.g., from a subject).

13. The method of claim 12, wherein the environmental sample comprises a water sample or a soil sample.

14. A method for detecting a target molecule in a sample, the method comprising using one or more of the components of the composition, kit, or system of claim 1, wherein the presence of the target molecule increases the rate of transcription and translation of the reporter molecule from the DNA transcription template.

15. A composition, kit, or system of detecting a target molecule comprising a chemical compound in a sample, the composition, kit, or system comprising (a) a biosensor molecule that modulates the expression of a target DNA sequence in a DNA transcription template, the biosensor molecule comprising SEQ ID NO: 47 or a variant thereof, and (b) one or more of the following components:
(i) the cellular transcription and translational machinery provided in a lysate from an engineered bacterial strain, or purified separately and reconstituted to defined concentrations;
(ii) a DNA transcription template whose expression is configured to be modulated by the biosensor molecule, and encoding a target DNA sequence, wherein the target DNA sequence comprises a reporter molecule.

16. The composition, kit, or system of claim 15 wherein one or more of the components are lyophilized.

17. A method of detecting a target molecule comprising a chemical compound in a biological or environmental sample, the method comprising:
(a) obtaining a biological or environmental sample which may or may not contain the target molecule and optionally concentrating and/or solubilizing the target molecule in the sample if necessary;
(b) adding the sample and/or the optionally concentrated and/or solubilized target molecule in the sample to a cell-free protein synthesis (CFPS) reaction, wherein if the target molecule is present in the sample then an output is generated (e.g., a visual, electronic, or optical output);
wherein the output is generated via steps that include: (i) the target molecule inducing expression from a DNA transcription template encoding a reporter molecule via a biosensor molecule, the biosensor molecule comprising SEQ ID NO: 47 or a variant thereof; and (ii) the reporter molecule generating an output either directly or indirectly; (iii) detecting the presence or absence of the target molecule based on the reporter molecule output.

18. The method of claim 17, wherein the cell-free protein synthesis reaction comprises:
(a) a cell extract from a host strain that (i) provides energy; (ii) provides cofactor regeneration; (iii) provides enzymes used for cell-free sensing of the target molecule; or (iv) any combination thereof; and
(b) exogenous supplied cell-free protein synthesis reagents not present in the cell extract that comprise at least one transcription template and a polymerase.

19. An engineered polypeptide comprising a variant of SEQ ID NO: 47, wherein the variant comprises one or more substitution mutations selected from M60A, M60L, M60F, P61A, P61N, D64A, D64L, D64K, D64C, D64F, K104A, K104V, K104H, L107A, L107Q, L107C, G128A, G128M, G128V, G128I, and G128F.

20. A cell-free transcription and translation system composition comprising the engineered polypeptide of claim 19.

21. The cell free transcription and translation system of claim 20, wherein the system is lyophilized.
